(19) United States
(12) Patent Application Publication
(10) Pub. No.: US 2012/0122981 A1
(43) Pub. Date: May 17, 2012

(54) Biomarkers Related to Insulin Resistance and Methods using the Same

(76) Inventors: Yun Fu Hu, Chapel Hill, NC (US); Costel Chirila, Durham, NC (US); Danny Alexander, Cary, NC (US); Michael Milburn, Cary, NC (US); Matthew W. Mitchell, Durham, NC (US); Walter Gall, Chapel Hill, NC (US); Kay A. Lawton, Raleigh, NC (US)

(21) Appl. No.: 13/258,780

(22) PCT Filed: Mar. 31, 2010
(86) PCT No.: PCT/US2010/029399

§ 371 Date: Jan. 19, 2012

Related U.S. Application Data
(60) Provisional application No. 61/165,336, filed on Mar. 31, 2009.
(60) Provisional application No. 61/166,572, filed on Apr. 3, 2009.

(51) Int. Cl. A61K 31/201; C12Q 1/25; C12Q 1/02; H01J 49/26; A61P 3/10; G01N 30/00; G01R 33/46; G01N 21/35; G01N 33/53; C07C 57/03

(52) U.S. Cl. 514/560; 435/7.92; 435/4; 435/29; 554/224; 73/61.52; 73/23.35; 324/307; 356/51; 250/282

(57) ABSTRACT

Biomarkers relating to glucose disposal rate, insulin resistance, and/or insulin resistance-related disorders are provided. Methods based on the same biomarkers are also provided.

### CROSS-REFERENCE TO RELATED APPLICATIONS

This application claims the benefit of U.S. Provisional Application Nos. 61/165,336, filed Mar. 31, 2009, and 61/166,572, filed Apr. 3, 2009; the entire contents of these applications are hereby incorporated by reference herein.

### FIELD

The invention generally relates to biomarkers correlated to glucose disposal and/or insulin resistance, methods for identifying biomarkers correlated to glucose disposal and/or insulin resistance and insulin resistance-related disorders and methods based on the same biomarkers.

### BACKGROUND

Diabetes is classified as either type 1 (early onset) or type 2 (adult onset), with type 2 comprising 90-95% of the cases of diabetes. Diabetes is the final stage in a disease process that begins to affect individuals long before the diagnosis of diabetes is made. Type 2 diabetes develops over 10 to 20 years and results from an impaired ability to utilize glucose (glucose utilization, glucose uptake in peripheral tissues) due to impaired sensitivity to insulin (insulin resistance).

Moreover, insulin resistance is central to development of a number of different diseases and conditions, such as nonalcoholic steatohepatitis (NASH), polycystic ovary syndrome (PCOS), cardiovascular disease, metabolic syndrome, and hypertension.

In pre-diabetes, insulin becomes less effective at helping tissues metabolize glucose. Pre-diabetics may be detectable as early as 20 years before diabetic symptoms become evident. Studies have shown that although patients show very few overt symptoms, long-term physiological damage is already occurring at this stage. Up to 60% of these individuals will progress to type 2 diabetes within 10 years.

The American Diabetes Association (ADA) has recommended routine screening to detect patients with pre-diabetes. Current screening methods for pre-diabetes include the fasting plasma glucose (FPG) test, the oral glucose tolerance test (OGTT), the fasting insulin test and the hyperinsulinemic euglycemic clamp (HI clamp). The first two tests are used clinically whereas the latter two tests are used extensively in research but rarely in the clinic. In addition, mathematical means (e.g., HOMA, QUICKI) that consider the fasting glucose and insulin levels together have been proposed. However, normal plasma insulin concentrations vary considerably between individuals as well as within an individual throughout the day. Further, these methods suffer from variability and methodological differences between laboratories and do not correlate rigorously with HI clamp studies.

Worldwide, an estimated 194 million adults have type 2 diabetes and this number is expected to increase to 333 million by 2025, largely due to the epidemic of obesity in westernized societies. In the United States, it is estimated that over 54 million adults are pre-diabetic. There are approximately 1.5 million new cases of type 2 diabetes a year in the United States. The annual US healthcare cost for diabetes is estimated at $174 billion. This figure has risen more than 32% since 2002. In industrialized countries such as the U.S., about 25% of medical expenditures treat glycemic control, 50% is associated with general medical care associated with diabetes, and the remaining 25% of the costs go to treat long-term complications, primarily cardiovascular disease. Considering the distribution of the healthcare costs and the fact that insulin resistance is a direct causal factor in cardiovascular disease and diabetes progression, it is no surprise that cardiovascular disease accounts for 70-80% of the mortality observed for diabetic patients. Detecting and preventing type 2 diabetes has become a major health care priority.

Diabetes may also lead to the development of other diseases or conditions, or is a risk factor in the development of conditions such as Metabolic Syndrome and cardiovascular diseases. Metabolic Syndrome is the clustering of a set of risk factors in an individual. According to the American Heart Association these risk factors include: abdominal obesity, decreased ability to properly process glucose (insulin resistance or glucose intolerance), dyslipidemia (high triglycerides, high LDL, low HDL cholesterol), hypertension, prothrombotic state (high fibrinogen or plasminogen activator inhibitor-1 in the blood) and proinflammatory state (elevated C-reactive protein in the blood). Metabolic Syndrome is also known as syndrome X, insulin resistance syndrome, obesity syndrome, dysmetabolic syndrome and Reaven's syndrome. Patients diagnosed with Metabolic Syndrome are at an increased risk of developing diabetes, cardiac and vascular disease. It is estimated that, in the United States, 20% of the adults (>50 million people) have metabolic syndrome. While it can affect anyone at any age, the incidence increases with increasing age and in individuals who are inactive, and significantly overweight, especially with excess abdominal fat.

Type 2 diabetes is the most common form of diabetes in the United States. According to the American Diabetes Foundation over 90% of the US diabetics suffer from Type 2 diabetes. Individuals with Type 2 diabetes have a combination of increased insulin resistance and decreased insulin secretion that combine to cause hyperglycemia. Most persons with Type 2 diabetes have Metabolic Syndrome.

The diagnosis for Metabolic Syndrome is based upon the clustering of three or more of the risk factors in an individual. A variety of medical organizations have definitions for the Metabolic Syndrome. The criteria proposed by the National Cholesterol Education Program (NCEP) Adult Treatment Panel III (ATP III), with minor modifications, are currently recommended and widely used in the United States.

The American Heart Association and the National Heart, Lung, and Blood Institute recommend that the metabolic syndrome be identified as the presence of three or more of these components: increased waist circumference (Men—equal to or greater than 40 inches (102 cm), Women—equal to or greater than 35 inches (88 cm); elevated triglycerides (equal to or greater than 150 mg/dL); reduced HDL (“good”) cholesterol (Men—less than 40 mg/dL, Women—less than 50 mg/dL); elevated blood pressure (equal to or greater than 130/85 mm Hg); elevated fasting glucose (equal to or greater than 100 mg/d₄

Type 2 diabetes develops slowly and often people first learn they have type 2 diabetes through blood tests done for another condition or as part of a routine exam. In some cases, type 2 diabetes may not be detected before damage to eyes, kidneys or other organs has occurred. A need exists for an objective, biochemical evaluation (e.g. lab test) that can be administered by a primary care provider to identify individuals that are at risk of developing Metabolic Syndrome or Type 2 diabetes.

Newer, more innovative molecular diagnostics that reflect the mechanisms of the patho-physiological progression to pre-diabetes and diabetes are needed because the prevalence of pre-diabetes and diabetes is increasing in global epidemic proportions. Mirroring the obesity epidemic, pre-diabetes and diabetes are largely preventable but are frequently undiagnosed or diagnosed too late due to the asymptomatic nature of the progression to clinical disease.

Although insulin resistance plays a central role in the development of numerous diseases, it is not readily detectable using many of the clinical measurements for pre-diabetic conditions. Insulin resistance develops prior to the onset of hyperglycemia and is associated with increased production of insulin. Over time (decades) the ability of the cell to respond to insulin decreases and the subject becomes resistant to the action of insulin (i.e., insulin resistant, IR). Eventually the beta-cells of the pancreas cannot produce sufficient insulin to compensate for the decreased insulin sensitivity and the beta-cells begin to lose function and apoptosis is triggered. Beta-cell function may be decreased as much as 80% in pre-diabetic subjects. As beta-cell dysfunction increases the production of insulin decreases resulting in lower insulin levels and high glucose levels in diabetic subjects. Vascular damage is associated with the increase in insulin resistance and the development of type 2 diabetes.

Therefore there is an unmet need for diagnostic biomarkers and tests that can identify insulin resistance and to determine the risk of disease progression in subjects with insulin resistance. Insulin resistance biomarkers and diagnostic tests can better identify and determine the risk of diabetes development in a pre-diabetic subject, can monitor disease development and progression and/or regression, can allow new therapeutic treatments to be developed and can be used to test therapeutic agents for efficacy on reversing insulin resistance and/or preventing insulin resistance and related diseases. Further, a need exists for diagnostic biomarkers to more effectively assess the efficacy and safety of pre-diabetic and diabetic therapeutic candidates.

### SUMMARY

In one embodiment, a method for diagnosing insulin resistance in a subject is provided comprising:

obtaining a biological sample from a subject;

analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, wherein at least the level of decanoyl carnitine or octanoyl carnitine is determined; and

comparing the level(s) of the one or more biomarkers in the sample to insulin resistance reference levels of the one or more biomarkers in order to diagnose whether the subject has insulin resistance.

In another embodiment, a method of classifying a subject as having normal insulin sensitivity or being insulin resistant is provided comprising:

analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, wherein at least the level of decanoyl carnitine or octanoyl carnitine is determined; and

comparing the level(s) of the one or more biomarkers in the sample to glucose disposal rate reference levels of the one or more biomarkers in order to classify the subject as having normal insulin sensitivity or being insulin resistant.

In a further embodiment, a method of determining susceptibility of a subject to type-2 diabetes is provided comprising:

analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, wherein at least the level of decanoyl carnitine or octanoyl carnitine is determined; and

comparing the level(s) of the one or more biomarkers in the sample to diabetes-positive and/or diabetes-negative reference levels of the one or more biomarkers in order to determine whether the subject is susceptible to developing type-2 diabetes.

In yet another embodiment, a method of monitoring the progression or regression of insulin resistance in a subject is provided comprising:

analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, wherein at least the level of decanoyl carnitine or octanoyl carnitine is determined; and

comparing the level(s) of the one or more biomarkers in the sample to insulin resistance progression and/or insulin resistance-regression reference levels of the one or more biomarkers in order to monitor the progression or regression of insulin resistance in the subject.

In yet another embodiment, a method of monitoring the efficacy of insulin resistance treatment is provided, comprising:

analyzing the biological sample from a subject to determine the level(s) of one or more biomarkers selected from the group consisting of decanoyl carnitine and octanoyl carnitine, and optionally one or more additional biomarkers selected from the group consisting of 2-hydroxybutyrate, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine;

treating the subject for insulin resistance;

analyzing a second biological sample from the subject to determine the level(s) of the one or more biomarkers, the second sample obtained from the subject at a second time point after treatment; and

comparing the level(s) of one or more biomarkers in the first sample to the level(s) of the one or more biomarkers in the second sample to assess the efficacy of the treatment for treating insulin resistance.

In yet a further embodiment, a method for predicting a subject's response to a course of treatment for insulin resistance is provided comprising:

analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, wherein at least the level of decanoyl carnitine or octanoyl carnitine is determined; and

comparing the level(s) of one or more biomarkers in the sample to treatment-positive and/or treatment-negative reference levels of the one or more biomarkers to predict whether the subject is likely to respond to a course of treatment.

In another embodiment, a method of monitoring insulin resistance in a bariatric patient is provided comprising:

analyzing a first biological sample from a subject having undergone bariatric surgery to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, the first sample obtained from the subject at a first time point after bariatric surgery;

analyzing a second biological sample from the subject to determine the level(s) of the one or more biomarkers, the second sample obtained from the subject at a second time point after the first time point; and

comparing the level(s) of one or more biomarkers in the first sample to the level(s) of the one or more biomarkers in the second sample to monitor insulin resistance in the subject.

In a further embodiment, a method for monitoring a subject's response to a course of treatment for insulin resistance is provided comprising:

analyzing a first biological sample from a subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, the first sample obtained from the subject at a first time point;

treating the subject for insulin resistance;

analyzing a second biological sample from the subject to determine the level(s) of the one or more biomarkers, the second sample obtained from the subject at a second time point after treatment; and

comparing the level(s) of one or more biomarkers in the first sample to the level(s) of the one or more biomarkers in the second sample to assess the efficacy of the treatment for treating insulin resistance.

In another embodiment, a method for determining a subject's probability of being insulin resistant is provided comprising:

obtaining a biological sample from a subject;

analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine,

predicting the glucose disposal rate in the subject by comparing the level(s) of the one or more biomarkers in the sample to glucose disposal rate reference levels of the one or more biomarkers;

comparing the predicted glucose disposal rate to an algorithm for insulin resistance based on the one or more markers; and

determining the probability that the subject is insulin resistant, thereby producing an insulin resistance score.

In yet another embodiment, a method of identifying an agent capable of modulating the level of a biomarker of insulin resistance is provided comprising:

analyzing a cell line from a subject at a first time point to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and lino leoyl lysophosphatidylcholine;

contacting the cell line with a test agent;

analyzing the cell line at a second time point to determine the level(s) of the one or more biomarkers, the second time point being a time after contacting with the test agent; and

comparing the level(s) of one or more biomarkers in the cell line at the first time point to the level(s) of the one or more biomarkers in the cell line at the second time point to identify an agent capable of modulating the level of the one or more biomarkers.

In a further embodiment, a method for predicting the glucose disposal rate in a subject is provided comprising:

analyzing the biological sample from the subject to determine the level(s) of one or more biomarkers selected from the group consisting one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, wherein at least the level of decanoyl carnitine or octanoyl carnitine is determined; and

comparing the levels of the one or more biomarkers in the sample to glucose disposal reference levels of the one or more biomarkers in order to predict the glucose disposal rate in the subject.

In another embodiment, a method for predicting the glucose disposal rate in a subject is provided comprising:

obtaining a biological sample from the subject;

determining the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine; and

analyzing the levels of the one or more biomarkers in the sample by a statistical analysis to predict the subject's glucose disposal rate.

In yet another embodiment, a method for determining the probability that a subject is insulin resistant is provided comprising:

obtaining a biological sample from the subject;

determining the level(s) of one or more biomarkers in the biological sample selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine; and

analyzing the levels of the one or more biomarkers in the sample by a statistical analysis to determine the probability that the subject is insulin resistant.

In a further embodiment, a method for measuring insulin resistance in a subject is provided comprising:

obtaining a biological sample from a subject;

analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, wherein at least the level of decanoyl carnitine or octanoyl carnitine is determined; and

using the determined levels of the level(s) of the one or more biomarkers and a reference model based on the one or more biomarkers to measure the insulin resistance in the subject.

In yet a further embodiment, a method of classifying a subject as having normal insulin sensitivity or being insulin resistant is provided comprising:

analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, wherein at least the level of decanoyl carnitine or octanoyl carnitine is determined; and

using the determined levels of the level(s) of the one or more biomarkers and a reference model based on the one or more biomarkers to classify the subject as having normal insulin sensitivity or being insulin resistant.

In a further embodiment, a method of determining susceptibility of a subject to type-2 diabetes is provided comprising:

analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, wherein at least the level of decanoyl carnitine or octanoyl carnitine is determined; and

using the determined levels of the level(s) of the one or more biomarkers and a reference model based on the one or more biomarkers to determine whether the subject is susceptible to developing type-2 diabetes.

In another embodiment, a method of monitoring the progression or regression of insulin resistance in a subject is provided comprising:

analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, wherein at least the level of decanoyl carnitine or octanoyl carnitine is determined; and

using the determined levels of the level(s) of the one or more biomarkers and a reference model based on the one or more biomarkers to monitor the progression or regression of insulin resistance in the subject.

In a further embodiment, a method of monitoring the efficacy of insulin resistance treatment is provided comprising:

analyzing the biological sample from a subject to determine the level(s) of one or more biomarkers selected from the group consisting of decanoyl carnitine and octanoyl carnitine, and optionally one or more additional biomarkers selected from the group consisting of 2-hydroxybutyrate, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine;

treating the subject for insulin resistance;

analyzing a second biological sample from the subject to determine the level(s) of the one or more biomarkers, the second sample obtained from the subject at a second time point after treatment; and

using the determined levels of the level(s) of the one or more biomarkers and a reference model based on the one or more biomarkers to assess the efficacy of the treatment for treating insulin resistance.

In another embodiment, a method for predicting a subject's response to a course of treatment for insulin resistance is provided comprising:

analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, wherein at least the level of decanoyl carnitine or octanoyl carnitine is determined;

using the determined levels of the level(s) of the one or more biomarkers and a reference model based on the one or more biomarkers to predict whether the subject is likely to respond to a course of treatment.

In yet another embodiment, a method of monitoring insulin resistance in a bariatric patient is provided comprising:

analyzing a first biological sample from a subject having undergone bariatric surgery to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, the first sample obtained from the subject at a first time point after bariatric surgery;

analyzing a second biological sample from the subject to determine the level(s) of the one or more biomarkers, the second sample obtained from the subject at a second time point after the first time point; and

using the determined levels of the level(s) of the one or more biomarkers and a reference model based on the one or more biomarkers to monitor insulin resistance in the subject.

In a further embodiment, a method for monitoring a subject's response to a course of treatment for insulin resistance is provided comprising:

analyzing a first biological sample from a subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, the first sample obtained from the subject at a first time point;

treating the subject for insulin resistance;

analyzing a second biological sample from the subject to determine the level(s) of the one or more biomarkers, the second sample obtained from the subject at a second time point after treatment;

using the determined levels of the level(s) of the one or more biomarkers and a reference model based on the one or more biomarkers to assess the efficacy of the treatment for treating insulin resistance.

In yet another embodiment, a method of identifying an agent capable of modulating insulin resistance is provided comprising:

analyzing a cell line from a subject at a first time point to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, linoleoyl lysophosphatidylcholine, and one or more biochemicals and/or metabolites in a pathway related to the one or more biomarkers;

contacting the cell line with a test agent;

analyzing the cell line at a second time point to determine the level(s) of the one or more biomarkers and/or one or more biochemicals and/or metabolites in a pathway related to the one or more biomarkers, the second time point being a time after contacting with the test agent;

comparing the level(s) of one or more biomarkers and/or biochemicals and/or metabolites in the cell line at the first time point to the level(s) of the one or more biomarkers and/or biochemicals and/or metabolites in the cell line at the second time point to identify an agent capable of modulating insulin resistance.

In a further embodiment, a method of treating an insulin resistant subject is provided comprising:

administering to the subject a therapeutic agent capable of modulating the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, linoleoyl lysophosphatidylcholine, and one or more biochemicals and/or metabolites in a pathway related to the one or more biomarkers.

In another embodiment, a method of classifying a subject as having normal glucose tolerance or having impaired glucose tolerance is provided comprising:

analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, and linoleoyl lysophosphatidylcholine, wherein at least the level of decanoyl carnitine or octanoyl carnitine is determined; and

comparing the level(s) of the one or more biomarkers in the sample to reference levels of the one or more biomarkers in order to classify the subject as having normal glucose tolerance or having impaired glucose tolerance.

### BRIEF DESCRIPTION OF THE DRAWINGS

FIG. 1A provides one example of using the model for predicting the probability that a subject has insulin resistance based on the subject's predicted glucose disposal rate (Rd, rate of disappearance). FIG. 1B provides one example of patient identification and selection for clinical trial in which the population of interest has at least a 70% probability of being insulin resistant.

FIG. 2 provides an example of a reference curve for determining the probability of insulin resistance. The exemplified predicted Rd values (calculated by the Rd regression model (i.e. Rd Predicted; x-axis) for nearly all subjects indicates insulin resistance, which was defined as Rd≦6.0 in this example.

FIG. 3 provides an example of a linear regression model and provides a correlation of actual and predicted Rd based on measuring biomarkers in plasma collected from a set of 401 insulin resistant subjects.

FIG. 4 provides an example of an ROC Curve based on one embodiment of the biomarkers used to generate the probability that a subject is insulin resistant.

FIG. 5 provides an example of the changes in predicted glucose disposal (Right panel) based on the biomarkers disclosed herein, which is in agreement with the actual glucose disposal as measured by the HI clamp (Left panel). C-Mur1, baseline prior to muraglitazar treatment; D-Mur2, following treatment with muraglitazar, a peroxisome proliferator-activated receptor agonist and an insulin sensitizer drug.

FIG. 6 shows predicted Rd in bariatric surgery subjects, where Pre-surgery is baseline prior to surgery and Post-surgery is after bariatric surgery, post-weight loss. The predicted Rd is consistent with measured Rd values and shows that the predicted Rd is low at baseline when subjects are insulin resistant and increases post-surgery when subjects are less insulin resistant/more insulin sensitive.

FIG. 7 shows Insulin Sensitivity and 2HB levels in bariatric surgery patients at baseline (A), before weight loss (B), and after weight loss (C).

FIG. 8 provides a schematic representation of one example of a biochemical pathway leading to the production of 2-hydroxybutyrate. It provides a schematic representation of one example of a biochemical pathway from 2HB to 2-ketobutyrate (2 KB) and the TCA cycle. It provides a schematic representation of a relationship between 2HB, the branched chain alpha-ketoacids and the TCA cycle.

FIG. 9 provides a heat map graphical representation of p-values obtained from t-test statistical analysis of the global biochemical profiling of metabolites measured in plasma collected from NGT-IS, NGT-IR, IGT, and IFG subjects. Columns 1-5 designate the following comparisons for each listed biomarker: 1, NGT-IS vs. NGT-IR; 2, NGT-IS vs. IGT; 3, NGT-IR vs. IGT; 4, NGT-IS vs. IFG; 5, IGT vs. IFG (white, most statistically significant (p≦1.0E-16); light grey (1.0E-16≦p≦0.001), dark grey (0.001≦p≦0.01), and black, not significant (p≧0.1)). As shown, FIG. 9A highlights organic acids and fatty acids, and FIG. 9B highlights carnitines and lyso-phospholipids. As shown in FIG. 9A, 2-FIB is useful for distinguishing NGT-IS from NGT-IR and NGT-IS from IGT; and a cluster of long-chain fatty acids such as palmitate that are useful for distinguishing NGT-IS from IGT. As shown in FIG. 9B, various acyl-carnitines and acyiglycerophosphocholines are useful for distinguishing NGT-IR and IGT from NGT-IS.

FIG. 10 provides a graphic representation of an example of the relationship of glucose tolerance as measured by the oral glucose tolerance test (OGTT) and insulin resistance.

FIG. 11 provides a graphic representation of an example of the relationship of glucose tolerance as measured by the fasting plasma glucose test (FPGT) and insulin resistance.

### DETAILED DESCRIPTION

The present invention relates to biomarkers correlated to glucose disposal rates and insulin resistance and related disorders (e.g. impaired fasting glucose, pre-diabetes, type-2 diabetes, etc.); methods for diagnosis of insulin resistance and related disorders; methods of determining predisposition to insulin resistance and related disorders; methods of monitoring progression/regression of insulin resistance and related disorders; methods of assessing efficacy of treatments and compositions for treating insulin resistance and related disorders; methods of screening compositions for activity in modulating biomarkers of insulin resistance and related disorders; methods of treating insulin resistance and related disorders; methods of identifying subjects for treatment with insulin resistant therapies; methods of identifying subjects for inclusion in clinical trials of insulin resistance therapies; as well as other methods based on biomarkers of insulin resistance and related disorders.

Current blood tests for insulin resistance perform poorly for early detection of insulin resistance or involve significant medical procedures.

In one embodiment, groups (also referred to as “panels”) of metabolites that can be used in a simple blood, urine, etc. test to predict insulin resistance are identified using metabolomic analysis. Such biomarkers correlate with insulin resistance at a level similar to, or better than, the correlation of glucose disposal rates as measured by the “gold standard” of measuring insulin resistance, the hyperinsulinemic euglycemic clamp.

Independent studies were carried out to identify a set of biomarkers that when used with a polynomic algorithm enables the early detection of changes in insulin resistance in a subject. The biomarkers of the instant disclosure can be used to provide a score indicating the probability of insulin resistance (“IR Score”) in a subject. The score can be based upon a clinically significant changed reference level for a biomarker and/or combination of biomarkers. The reference level can be derived from an algorithm or computed from indices for impaired glucose tolerance and can be presented in a report. The IR Score places the subject in the range of insulin resistance from normal (insulin sensitive) to high and/or can be used to determine a probability that the subject has insulin resistance. Disease progression or remission can be monitored by periodic determination and monitoring of the IR Score. Response to therapeutic intervention can be determined by monitoring the IR Score. The IR Score can also be used to evaluate drug efficacy or to identify subjects to be treated with insulin resistance therapies, such as insulin sensitizers, or to identify subjects for inclusion in clinical trials.

Prior to describing this invention in further detail, however, the following terms will first be defined.

### Definitions:

“Biomarker” means a compound, preferably a metabolite, that is differentially present (i.e., increased or decreased) in a biological sample from a subject or a group of subjects having a first phenotype (e.g., having a disease) as compared to a biological sample from a subject or group of subjects having a second phenotype (e.g., not having the disease). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using either Welch's T-test or Wilcoxon's rank-sum Test). Alternatively, the biomarkers demonstrate a correlation with insulin resistance, or particular levels or stages of insulin resistance. The range of possible correlations is between negative (−) 1 and positive (+) 1. A result of negative (−) 1 means a perfect negative correlation and a positive (+) 1 means a perfect positive correlation, and 0 means no correlation at all. A “substantial positive correlation” refers to a biomarker having a correlation from +0.25 to +1.0 with a disorder or with a clinical measurement (e.g., Rd), while a “substantial negative correlation” refers to a correlation from −0.25 to −1.0 with a given disorder or clinical measurement. A “significant positive correlation” refers to a biomarker having a correlation of from +0.5 to +1.0 with a given disorder or clinical measurement (e.g., Rd), while a “significant negative correlation” refers to a correlation to a disorder of from −0.5 to −1.0 with a given disorder or clinical measurement.

The “level” of one or more biomarkers means the absolute or relative amount or concentration of the biomarker in the sample.

“Sample” or “biological sample” or “specimen” means biological material isolated from a subject. The biological sample may contain any biological material suitable for detecting the desired biomarkers, and may comprise cellular and/or non-cellular material from the subject. The sample can be isolated from any suitable biological tissue or fluid such as, for example, adipose tissue, aortic tissue, liver tissue, blood, blood plasma, saliva, serum, cerebrospinal fluid, cystic fluid, exudates, or urine.

“Subject” means any animal, but is preferably a mammal, such as, for example, a human, monkey, non-human primate, rat, mouse, cow, dog, cat, pig, horse, or rabbit.

A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype. For example, an “insulin resistance-positive reference level” of a biomarker means a level of a biomarker that is indicative of a positive diagnosis of insulin resistance in a subject, and an “insulin resistance-negative reference level” of a biomarker means a level of a biomarker that is indicative of a negative diagnosis of insulin resistance in a subject. As another example, an “insulin resistance-progression-positive reference level” of a biomarker means a level of a biomarker that is indicative of progression of insulin resistance in a subject, and an “insulin resistance-regression-positive reference level” of a biomarker means a level of a biomarker that is indicative of regression of insulin resistance. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. A “reference level” may also be a “standard curve reference level” based on the levels of one or more biomarkers determined from a population and plotted on appropriate axes to produce a reference curve (e.g. a standard probability curve). Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). A standard curve reference level may be determined from a group of reference levels from a group of subjects having a particular disease state, phenotype, or lack thereof (e.g. known glucose disposal rates) using statistical analysis, such as univariate or multivariate regression analysis, logistic regression analysis, linear regression analysis, and the like of the levels of such biomarkers in samples from the group. Such reference levels may also be tailored to specific techniques that are used to measure levels of biomarkers in biological samples (e.g., LC-MS, GC-MS, NMR, enzyme assays, etc.), where the levels of biomarkers may differ based on the specific technique that is used.

“Non-biomarker compound” means a compound that is not differentially present in a biological sample from a subject or a group of subjects having a first phenotype (e.g., having a first disease) as compared to a biological sample from a subject or group of subjects having a second phenotype (e.g., not having the first disease). Such non-biomarker compounds may, however, be biomarkers in a biological sample from a subject or a group of subjects having a third phenotype (e.g., having a second disease) as compared to the first phenotype (e.g., having the first disease) or the second phenotype (e.g., not having the first disease).

“Metabolite”, or “small molecule”, means organic and inorganic molecules which are present in a cell. The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000). The small molecules of the cell are generally found free in solution in the cytoplasm or in other organelles, such as the mitochondria, where they form a pool of intermediates which can be metabolized further or used to generate large molecules, called macromolecules. The term “small molecules” includes signaling molecules and intermediates in the chemical reactions that transform energy derived from food into usable forms. Examples of small molecules include sugars, fatty acids, amino acids, nucleotides, intermediates formed during cellular processes, and other small molecules found within the cell.

“Metabolic profile”, or “small molecule profile”, means a complete or partial inventory of small molecules within a targeted cell, tissue, organ, organism, or fraction thereof (e.g., cellular compartment). The inventory may include the quantity and/or type of small molecules present. The “small molecule profile” may be determined using a single technique or multiple different techniques.

“Metabolome” means all of the small molecules present in a given organism.

“Diabetes” refers to a group of metabolic diseases characterized by high blood sugar (glucose) levels which result from defects in insulin secretion or action, or both.

“Type 2 diabetes” refers to one of the two major types of diabetes, the type in which the beta cells of the pancreas produce insulin, at least in the early stages of the disease, but the body is unable to use it effectively because the cells of the body are resistant to the action of insulin. In later stages of the disease the beta cells may stop producing insulin. Type 2 diabetes is also known as insulin-resistant diabetes, non-insulin dependent diabetes and adult-onset diabetes.

“Pre-diabetes” refers to one or more early diabetes-related conditions including impaired glucose utilization, abnormal or impaired fasting glucose levels, impaired glucose tolerance, impaired insulin sensitivity and insulin resistance.

“Insulin resistant” refers to the condition when cells become resistant to the effects of insulin—a hormone that regulates the uptake of glucose into cells—or when the amount of insulin produced is insufficient to maintain a normal glucose level. Cells are diminished in the ability to respond to the action of insulin in promoting the transport of the sugar glucose from blood into muscles and other tissues (i.e. sensitivity to insulin decreases). Eventually, the pancreas produces far more insulin than normal and the cells continue to be resistant. As long as enough insulin is produced to overcome this resistance, blood glucose levels remain normal. Once the pancreas is no longer able to keep up, blood glucose starts to rise, resulting in diabetes. Insulin resistance ranges from normal (insulin sensitive) to insulin resistant (IR).

“Insulin sensitivity” refers to the ability of cells to respond to the effects of insulin to regulate the uptake and utilization of glucose. Insulin sensitivity ranges from normal (insulin sensitive) to Insulin Resistant (IR).

The “IR Score” is a measure of the probability of insulin resistance in a subject based upon the predicted glucose disposal rate calculated using the insulin resistance biomarkers (e.g. along with models and/or algorithms) that will allow a physician to determine the probability that a subject is insulin resistant.

“Glucose utilization” refers to the absorption of glucose from the blood by muscle and fat cells and utilization of the sugar for cellular metabolism. The uptake of glucose into cells is stimulated by insulin.

“Rd” refers to glucose disposal rate (Rate of disappearance of glucose), a metric for glucose utilization. The rate at which glucose disappears from the blood (disposal rate) is an indication of the ability of the body to respond to insulin (i.e. insulin sensitive). There are several methods to determine Rd and the hyperinsulinemic euglycemic clamp is regarded as the “gold standard” method. In this technique, while a fixed amount of insulin is infused, the blood glucose is “clamped” at a predetermined level by the titration of a variable rate of glucose infusion. The underlying principle is that upon reaching steady state, by definition, glucose disposal is equivalent to glucose appearance. During hyperinsulinemia, glucose disposal (Rd) is primarily accounted for by glucose uptake into skeletal muscle, and glucose appearance is equal to the sum of the exogenous glucose infusion rate plus the rate of hepatic glucose output (HGO). The rate of glucose infusion during the last 30 minutes of the test determines insulin sensitivity. If high levels of glucose (Rd=7.5 mg/kg/min or higher) are required, the patient is insulin-sensitive. Very low levels (Rd=4.0 mg/kg/min or lower) of required glucose indicate that the body is resistant to insulin action. Levels between 4.0 and 7.5 mg/kg/min (Rd values between 4.0 mg/kg/min and 7.5 mg/kg/min) of required glucose are not definitive and suggest sensitivity to insulin is impaired and that the subject may have “impaired glucose tolerance,” which may sometimes be a sign of insulin resistance.

“Mffm” and “Mwbm” refer to glucose disposal rate (M) calculated as the mean rate of glucose infusion during the past 60 minutes of the clamp examination (steady state) and expressed as milligrams per minute per kilogram of fat free mass (ffm) or whole body mass (wbm). Subjects with an Mffm less than 45 umol/min/kg ffm are generally regarded as insulin resistant. Subjects with an Mwbm of less than 5.6 mg/kg/min are generally regarded as insulin resistant.

“Dysglycemia” refers to disturbed blood sugar (i.e. glucose) regulation and results in abnormal blood glucose levels from any cause that contributes to disease. Subjects having higher than normal levels of blood sugar are considered “hyperglycemic” while subjects having lower than normal levels of blood sugar are considered “hypoglycemic”.

“Impaired fasting glucose (IFG)” and “impaired glucose tolerance (IGT)” are the two clinical definitions of “pre-diabetes”. IFG is defined as a fasting blood glucose concentration of 100-125 mg/dL. IGT is defined as a postprandial (after eating) blood glucose concentration of 140-199 mg/dL. It is known that IFG and IGT do not always detect the same pre-diabetic populations. Between the two populations there is approximately a 60% overlap observed. Fasting plasma glucose levels are a more efficient means of inferring a patient's pancreatic function, or insulin secretion, whereas postprandial glucose levels are more frequently associated with inferring levels of insulin sensitivity or resistance. IGT is known to identify a greater percentage of the pre-diabetic population compared to IFG. The IFG condition is associated with lower insulin secretion, whereas the IGT condition is known to be strongly associated with insulin resistance. Numerous studies have been carried out that demonstrate that IGT individuals with normal FPG values are at increased risk for cardiovascular disease. Patients with normal FPG values may have abnormal postprandial glucose values and are often unaware of their risk for pre-diabetes, diabetes, and cardiovascular disease.

“Fasting plasma glucose (FPG) test” is a simple test measuring blood glucose levels after an 8 hour fast. According to the ADA, blood glucose concentration of 100-125 mg/dL is considered IFG and defines pre-diabetes whereas ≧126 mg/dL defines diabetes. As stated by the ADA, FPG is the preferred test to diagnose diabetes and pre-diabetes due to its ease of use, patient acceptability, lower cost, and relative reproducibility. The weakness in the FPG test is that patients are quite advanced toward Type 2 Diabetes before fasting glucose levels change.

“Oral glucose tolerance test (OGTT)”, a dynamic measurement of glucose, is a postprandial measurement of a patient's blood glucose levels after oral ingestion of a 75 g glucose drink. Traditional measurements include a fasting blood sample at the beginning of the test, a one hour time point blood sample, and a 2 hour time point blood sample. A patient's blood glucose concentration at the 2 hour time point defines the level of glucose tolerance: Normal glucose tolerance (NGT)≦140 mg/dL blood glucose; Impaired glucose tolerance (IGT)=140-199 mg/dL blood glucose; Diabetes ≧200 mg/dL blood glucose. As stated by the ADA, even though the OGTT is known to be more sensitive and specific at diagnosing pre-diabetes and diabetes, it is not recommended for routine clinical use because of its poor reproducibility and difficulty to perform in practice.

“Fasting insulin test” measures the circulating mature form of insulin in plasma. The current definition of hyperinsulinemia is difficult due to lack of standardization of insulin immunoassays, cross-reactivity to proinsulin forms, and no consensus on analytical requirements for the assays. Within-assay CVs range from 3.7%-39% and among-assay CVs range from 12%-66%. Therefore, fasting insulin is not commonly measured in the clinical setting and is limited to the research setting.

The “hyperinsulinemic euglycemic clamp (HI clamp)” is considered worldwide as the “gold standard” for measuring insulin resistance in patients. It is performed in a research setting, requires insertion of two catheters into the patient and the patient must remain immobilized for up to six hours. The HI clamp involves creating steady-state hyperinsulinemia by insulin infusion, along with parallel glucose infusion in order to quantify the required amount of glucose to maintain euglycemia (normal concentration of glucose in the blood; also called normoglycemia). The result is a measure of the insulin-dependent glucose disposal rate (Rd), measuring the peripheral uptake of glucose by the muscle (primarily) and adipose tissues. This rate of glucose uptake is notated by M, whole body glucose metabolism by insulin action under steady state conditions. Therefore, a high M indicates high insulin sensitivity and a lower M value indicates reduced insulin sensitivity, i.e. insulin resistant. The HI clamp requires three trained professionals to carry out the procedure, including simultaneous infusions of insulin and glucose over 2-4 hours and frequent blood sampling every 5 minutes for analysis of insulin and glucose levels. Due to the high cost, complexity, and time required for the HI clamp, this procedure is strictly limited to the clinical research setting.

“Obesity” refers to a chronic condition defined by an excess amount body fat. The normal amount of body fat (expressed as percentage of body weight) is between 25-30% in women and 18-23% in men. Women with over 30% body fat and men with over 25% body fat are considered obese.

“Body Mass Index, (or BMI)” refers to a calculation that uses the height and weight of an individual to estimate the amount of the individual's body fat. Too much body fat (e.g. obesity) can lead to illnesses and other health problems. BMI is the measurement of choice for many physicians and researchers studying obesity. BMI is calculated using a mathematical formula that takes into account both height and weight of the individual. BMI equals a person's weight in kilograms divided by height in meters squared. (BMI=kg/m²). Subjects having a BMI less than 19 are considered to be underweight, while those with a BMI of between 19 and 25 are considered to be of normal weight, while a BMI of between 25 to 29 are generally considered overweight, while individuals with a BMI of 30 or more are typically considered obese. Morbid obesity refers to a subject having a BMI of 40 or greater.

“Insulin resistance related disorders” refers to diseases, disorders or conditions that are associated with (e.g., co-morbid) or increased in prevalence in subjects that are insulin resistant. For example, atherosclerosis, coronary artery disease, myocardial infarction, myocardial ischemia, dysglycemia, hypertension, metabolic syndrome, polycystic ovary syndrome, neuropathy, nephropathy, chronic kidney disease, fatty liver disease and the like.

### I. Biomarkers

The biomarkers described herein were discovered using metabolomic profiling techniques. Such metabolomic profiling techniques are described in more detail in the Examples set forth below as well as in U.S. Pat. Nos. 7,005,255 and 7,329,489 and U.S. Pat. No. 7,635,556, U.S. Pat. No. 7,682,783, U.S. Pat. No. 7,682,784, and U.S. Pat. No. 7,550,258, the entire contents of all of which are hereby incorporated herein by reference.

Generally, metabolic profiles may be determined for biological samples from human subjects diagnosed with a condition such as being insulin resistant as well as from one or more other groups of human subjects (e.g., healthy control subjects with normal glucose tolerance, subjects with impaired glucose tolerance, subjects with insulin resistance, or having known glucose disposal rates). The metabolic profile for insulin resistance or an insulin resistance-related disorder may then be compared to the metabolic profile for biological samples from the one or more other groups of subjects. The comparisons may be conducted using models or algorithms, such as those described herein. Those molecules differentially present, including those molecules differentially present at a level that is statistically significant, in the metabolic profile of samples from subjects being insulin resistant or having a related disorder as compared to another group (e.g., healthy control subjects being insulin sensitive) may be identified as biomarkers to distinguish those groups.

Biomarkers for use in the methods disclosed herein may be obtained from any source of biomarkers related to glucose disposal, insulin resistance and/or pre-diabetes. Biomarkers for use in methods disclosed herein relating to insulin resistance include those listed in Table 4, and subsets thereof. In one embodiment, the biomarkers include decanoyl carnitine and/or octanoyl carnitine in combination with one or more additional biomarkers listed in Table 4, such as 2-hydroxybutyrate, oleic acid, and linoleoyl LPC, palmitate, stearate, and combinations thereof. Additional biomarkers for use in combination with those disclosed herein include those disclosed in International Patent Application Publication No. WO 2009/014639 and U.S. application Ser. No. 12/218,980, filed Jul. 17, 2008, the entireties of which are hereby incorporated by reference herein. In one aspect, the biomarkers correlate to insulin resistance.

Biomarkers for use in methods disclosed herein correlating to glucose disposal, insulin resistance and related disorders or conditions, such as being impaired insulin sensitive, insulin resistant, or pre-diabetic include one or more of those listed in Table 4. Such biomarkers allow subjects to be classified as insulin resistant, insulin impaired, or insulin sensitive. Any of the biomarkers listed in Table 4 (alone or in combination) can be used in the methods disclosed herein. In addition, any combination of two or more biomarkers listed in Table 4 can be used; for example, biomarkers such as decanoyl carnitine or octanoyl carnitine can be used in combination with one or more additional biomarkers listed in Table 4 (e.g., 2-hydroxybutyrate, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl-LPC, palmitate, palmitoleic acid, palmitoyl-LPC, serine, stearate, threonine, tryptophan, linoleoyl-LPC, 1,5-anhydroglucitol, stearoyl-LPC, glutamyl valine, gamma-glutamyl-leucine, heptadecenoic acid, alpha-ketobutyrate, cysteine, urate) in any of the disclosed methods. In another embodiment, biomarkers such as decanoyl carnitine or octanoyl carnitine can be combined with 2-hydroxybutyrate for use in any of the methods disclosed herein. Furthermore, such combinations of decanoyl carnitine or octanoyl carnitine with 2-hydroxybutyrate can be further combined with one or more additional biomarkers listed in Table 4 for use in the methods disclosed here. In one embodiment, the biomarkers for use in the disclosed methods include a combination of 2-hydroxybutyrate, decanoyl carnitine, linoleoyl-LPC, creatine, and palmitate. In another embodiment, the biomarkers for use in the disclosed methods include a combination of 2-hydroxybutyrate, decanoyl carnitine, linoleoyl-LPC, creatine, and stearate. Such combinations can also be combined with clinical measurements or predictors of insulin resistance, such as body mass index, fasting plasma insulin or C-peptide measurements. Examples of additional combinations that can be used in the methods disclosed herein include those provided in the Examples below.

In one embodiment, biomarkers for use in distinguishing or aiding in distinguishing, between subjects being impaired insulin sensitive from subjects not having impaired insulin sensitivity include one or more of those listed Table 4. In another aspect, biomarkers for use in diagnosing a subject as being insulin resistant include one or more of those listed Table 4. In another example, biomarkers for use in distinguishing subjects being insulin resistant from subjects not being insulin resistant include one or more of those listed Table 4. In still another example, biomarkers for use in distinguishing subjects being insulin resistant from subjects being insulin sensitive include one or more of those listed in Table 4. In another example, biomarkers for use in categorizing, or aiding in categorizing, a subject as having impaired fasting glucose levels or impaired glucose tolerance include one or more of those listed Table 4. In another example, biomarkers for use in identifying subjects for treatment by the administration of insulin resistance therapeutics include one or more of those listed in Table 4. In still another example, biomarkers for use in identifying subjects for admission into clinical trials for the administration of test compositions for effectiveness in treating insulin resistance or related conditions, include one or more of those listed in Table 4.

Additional biomarkers for use in the methods disclosed herein include metabolites related to the biomarkers listed in Table 4. In addition, such additional biomarkers may also be useful in combination with the biomarkers in Table 4 for example as ratios of biomarkers and such additional biomarkers. Such metabolites may be related by proximity in a given pathway, or in a related pathway or associated with related pathways. Biochemical pathways related to one or more biomarkers listed in Table 4 include pathways involved in the formation of such biomarkers, pathways involved in the degradation of such biomarkers, and/or pathways in which the biomarkers are involved. For example, one biomarker listed in Table 4 is 2-hydroxybutyrate. Additional biomarkers for use in the methods of the present invention relating the 2-hydroxybutyrate include any of the enzymes, cofactors, genes, or the like involved in 2-hydroxybutyrate formation, metabolism, or utilization. For example, potential biomarkers from the 2-hydroxybutyrate formation pathway include, lactate dehydrogenase, hydroxybutyric acid dehydrogenase, alanine transaminase, gamma-cystathionase, branched-chain alpha-keto acid dehydrogenase, and the like. The substrates, intermediates, and enzymes in this pathway and related pathways may also be used as biomarkers for glucose disposal and/or insulin resistance. For example, additional biomarkers related to 2-hydroxybutyrate include lactate dehydrogenase (LDH) or activation of hydroxybutyric acid dehydrogenase (HBDH) or branched chain alpha-keto acid dehydrogenase (BCKDH). In another embodiment, a pathway in which 2-hydroxybutyrate is involved is the citrate pathway (TCA pathway). When flux into the TCA cycle is reduced, there is typically an overflow of 2-hydroxybutyrate. Thus, any of the enzymes, co-factors, genes, and the like involved in the TCA cycle may also be biomarkers for glucose disposal, insulin resistance and related disorders. In addition, ratios of such enzymes, co-factors, genes and the like involved with such pathways with the biomarker 3-hydroxy-butyrate, 3-methyl-2-oxo-butyric acid, arginine, betaine, creatine, decanoyl carnitine, docosatetraenoic acid, glutamic acid, glycine, linoleic acid, linolenic acid, margaric acid, octanoyl carnitine, oleic acid, oleoyl-LPC, palmitate, palmitoleic acid, palmitoyl-LPC, serine, stearate, threonine, tryptophan, linoleoyl-LPC, 1,5-anhydroglucitol, stearoyl-LPC, glutamyl valine, gamma-glutamyl-leucine, heptadecenoic acid, alpha-ketobutyrate, cysteine, urate may also find use in the methods disclosed herein.

In addition, metabolites and pathways related to the biomarkers listed in Table 4 may be useful as sources of additional biomarkers for insulin resistance. For example, metabolites and pathways related to 2-hydroxybutyrate may also be biomarkers of insulin resistance, such as alpha-ketoacids, 3-methyl-2-oxobutyrate and 3-methyl-2-oxovalerate. Furthermore, other metabolites and agents involved in branched chain alpha-keto acid biosynthesis, metabolism, and utilization may also be useful as biomarkers of insulin resistance or related conditions.

Any number of biomarkers may be used in the methods disclosed herein. That is, the disclosed methods may include the determination of the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, fifteen or more biomarkers, etc., including a combination of all of the biomarkers in Table 4. In another aspect, the number of biomarkers for use in the disclosed methods include the levels of about twenty-five or less biomarkers, twenty or less, fifteen or less, ten or less, nine or less, eight or less, seven or less, six or less, or five or less biomarkers. In another aspect, the number of biomarkers for use in the disclosed methods include the levels of one, two, three, four, five, six, seven, eight, nine, ten, eleven, twelve, thirteen, fourteen, fifteen, twenty, or twenty-five biomarkers. Examples of specific combinations of biomarkers (and in some instances additional variables) that can be used in any of the methods disclosed herein are disclosed in the Examples (e.g., the models discussed in the Examples include specific combinations of biomarkers). The biomarkers may be used with or without the additional variables presented in the specific models.

The biomarkers disclosed herein may also be used to generate an insulin resistance score (“IR Score”) to predict a subject's glucose disposal rate or probability of being insulin resistant for use in any of the disclosed methods. Any method or algorithm can be used to generate an IR Score based on the biomarkers in Table 4 for use in the methods of the present disclosure. Such methods and algorithms include those provided in the Examples below, such as Example 3.

The biomarkers, panels, and algorithms may provide sensitivity levels for detecting or predicting glucose disposal and/or insulin resistance greater than conventional methods, such as the oral glucose tolerance test, fasting plasma glucose test, hemoglobin A1C (and estimated average glucose, eAG), fasting plasma insulin, fasting proinsulin, adiponectin, HOMA-IR, and the like. In some embodiments, the biomarkers, panels, and algorithms provided herein provide sensitivity levels greater than about 55%, 56%, 57%, 58%, 59%, 60% or greater.

In other embodiments, the biomarkers, panels, and algorithms disclosed herein may provide a specificity level for detecting or predicting glucose disposal and/or insulin resistance in a subject greater than conventional methods such as the oral glucose tolerance test, fasting plasma glucose test, adiponectin, and the like. In some embodiments, the biomarkers, panels, and algorithms provided herein provide specificity levels greater than about 80%, 85%, 90%, or greater.

In addition, the methods disclosed herein using the biomarkers and models listed in the tables may be used in combination with clinical diagnostic measures of the respective conditions. Combinations with clinical diagnostics (such as oral glucose tolerance test, fasting plasma glucose test, free fatty acid measurement, hemoglobin A1C (and estimated average glucose, eAG) measurements, fasting plasma insulin measurements, fasting proinsulin measurements, fasting C-peptide measurements, glucose sensitivity (beta cell index) measurements, adiponectin measurements, uric acid measurements, systolic and diastolic blood pressure measurements, triglyceride measurements, triglyceride/HDL ratio, cholesterol (HDL, LDL) measurements, LDL/HDL ratio, waist/hip ratio, age, family history of diabetes (T1D and/or T2D), family history of cardiovascular disease) may facilitate the disclosed methods, or confirm results of the disclosed methods, (for example, facilitating or confirming diagnosis, monitoring progression or regression, and/or determining predisposition to pre-diabetes).

Any suitable method may be used to detect the biomarkers in a biological sample in order to determine the level(s) of the one or more biomarkers. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof (e.g. LC-MS-MS). Further, the level(s) of the one or more biomarkers may be detected indirectly, for example, by using an assay that measures the level of a compound (or compounds) that correlates with the level of the biomarker(s) that are desired to be measured.

In some embodiments, the biological samples for use in the detection of the biomarkers are transformed into analytical samples prior to the analysis of the level or detection of the biomarker in the sample. For example, in some embodiments, protein extractions may be performed to transform the sample prior to analysis by, for example, liquid chromatography (LC) or tandem mass spectrometry (MS-MS), or combinations thereof. In other embodiments, the samples may be transformed during the analysis, for example by tandem mass spectrometry methods.

### II. Diagnostic Methods

The biomarkers described herein may be used to diagnose, or to aid in diagnosing, whether a subject has a disease or condition, such as being insulin resistant, or having an insulin resistance-related disorder (e.g., dysglycemia). For example, biomarkers for use in diagnosing, or aiding in diagnosing, whether a subject is insulin resistant include one or more of those identified biomarkers Table 4. In one embodiment, the biomarkers include one or more of those identified in Table 4 and combinations thereof. Any biomarker listed in Table 4 may be used in the diagnostic methods, as well as any combination of the biomarkers listed in Table 4. In one embodiment the biomarkers include decanoyl carnitine or octanoyl carnitine. In another example, the biomarkers include decanoyl carnitine or octanoyl carnitine in combination with any other biomarker, such as those listed 2-hydroxybutyrate, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl-LPC, palmitate, palmitoleic acid, palmitoyl-LPC, serine, stearate, threonine, tryptophan, linoleoyl-LPC, 1,5-anhydroglucitol, stearoyl-LPC, glutamyl valine, gamma-glutamyl-leucine, heptadecenoic acid, alpha-ketobutyrate, cysteine, urate, including oleic acid, linoleoyl LPC, 2-hydroxybutyrate, palmitate, creatine, or combinations thereof. In another embodiment, combinations of biomarkers include those, such as decanoyl carnitine or octanoyl carnitine in combination with 2-hydroxybutyrate in further combination with any other biomarker identified 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl-LPC, palmitate, palmitoleic acid, palmitoyl-LPC, serine, stearate, threonine, tryptophan, linoleoyl-LPC, 1,5-anhydroglucitol, stearoyl-LPC, glutamyl valine, gamma-glutamyl-leucine, heptadecenoic acid, alpha-ketobutyrate, cysteine, urate.

Methods for diagnosing, or aiding in diagnosing, whether a subject has a disease or condition, such as being insulin resistant or having an insulin resistance related disorder, may be performed using one or more of the biomarkers identified in Table 4. A method of diagnosing (or aiding in diagnosing) whether a subject has a disease or condition, such as being insulin resistant or pre-diabetic, comprises (1) analyzing a biological sample from a subject to determine the level(s) of one or more biomarkers of insulin resistance listed in Table 4 in the sample and (2) comparing the level(s) of the one or more biomarkers in the sample to insulin-resistance-positive and/or insulin-resistance-negative reference levels of the one or more biomarkers in order to diagnose (or aid in the diagnosis of) whether the subject is insulin resistant. When such a method is used in aiding in the diagnosis of a disease or condition, such as insulin resistance or pre-diabetes, the results of the method may be used along with other methods (or the results thereof) useful in the clinical determination of whether a subject has a given disease or condition. Methods useful in the clinical determination of whether a subject has a disease or condition such as insulin resistance or pre-diabetes are known in the art. For example, methods useful in the clinical determination of whether a subject is insulin resistant or is at risk of being insulin resistant include, for example, glucose disposal rates (Rd, M-wbm, M-ffm), body weight measurements, waist circumference measurements, BMI determinations, waist/hip ratio, triglycerides measurements, cholesterol (HDL, LDL) measurements, LDL/HDL ratio, triglyceride/HDL ratio, age, family history of diabetes (T1D and/or T2D), family history of cardiovascular disease, Peptide YY measurements, C-peptide measurements, Hemoglobin A1C measurements and estimated average glucose, (eAG), adiponectin measurements, fasting plasma glucose measurements (e.g., oral glucose tolerance test, fasting plasma glucose test), free fatty acid measurements, fasting plasma insulin and pro-insulin measurements, systolic and diastolic blood pressure measurements, urate measurements and the like. Methods useful for the clinical determination of whether a subject has insulin resistance include the hyperinsulinemic euglycemic clamp (HI clamp).

In another example, the identification of biomarkers for diseases or conditions such as insulin resistance or pre-diabetes allows for the diagnosis of (or for aiding in the diagnosis of) such diseases or conditions in subjects presenting one or more symptoms of the disease or condition. For example, a method of diagnosing (or aiding in diagnosing) whether a subject has insulin resistance comprises (1) analyzing a biological sample from a subject presenting one or more symptoms of insulin resistance to determine the level(s) of one or more biomarkers of insulin resistance selected from the biomarkers listed in Table 4, in the sample and (2) comparing the level(s) of the one or more biomarkers in the sample to insulin resistance-positive and/or insulin resistance-negative reference levels of the one or more biomarkers in order to diagnose (or aid in the diagnosis of) whether the subject has insulin resistance. The biomarkers for insulin resistance may also be used to classify subjects as being either insulin resistant, insulin sensitive, or having impaired insulin sensitivity. As described in Example 2 below, biomarkers were identified that may be used to classify subjects as being insulin resistant, insulin sensitive, or having impaired insulin sensitivity. The biomarkers in Table 4 may also be used to classify subjects as having impaired fasting glucose levels or impaired glucose tolerance or normal glucose tolerance (e.g., Example 12 shows classification of subjects as having either impaired glucose tolerance or normal glucose tolerance based on measurement of levels of certain biomarkers). Thus, the biomarkers may indicate compounds that increase and decrease as the glucose disposal rate increases. By determining appropriate reference levels of the biomarkers for each group (insulin resistant, insulin impaired, insulin sensitive), subjects can be diagnosed appropriately. The results of this method may be combined with the results of clinical measurements to aid in the diagnosis of insulin resistance or related disorders.

Increased insulin resistance correlates with the glucose disposal rate (Rd) as measured by the HI clamp. As exemplified below, metabolomic analysis was carried out to identify biomarkers that correlate with the glucose disposal rate (Rd). These biomarkers can be used in a mathematical model to determine the glucose disposal rate of the subject. The insulin sensitivity of the individual can be determined using this model. Using metabolomic analysis, panels of metabolites, such as those provided in Table 4 that can be used in a simple blood test to predict insulin resistance as measured by the “gold standard” of hyperinsulinemic euglycemic clamps were discovered.

Independent studies were carried out to identify a set of biomarkers that when used with a polynomic algorithm enables the early detection of changes in insulin resistance in a subject. In one aspect, the biomarkers provided herein can be used to provide a physician with a probability score (“IR Score”) indicating the probability that a subject is insulin resistant. The score is based upon clinically significant changed reference level(s) for a biomarker and/or combination of biomarkers. The reference level can be derived from an algorithm or computed from indices for impaired glucose disposal. The IR Score places the subject in the range of insulin resistance from normal (i.e. insulin sensitive) to insulin resistant to highly resistant. Disease progression or remission can be monitored by periodic determination and monitoring of the IR Score. Response to therapeutic intervention can be determined by monitoring the IR Score. The IR Score can also be used to evaluate drug efficacy.

Thus, the disclosure also provides methods for determining a subject's insulin resistance score (IR score) that may be performed using one or more of the biomarkers identified in Table 4 in the sample, and (2) comparing the level(s) of the one or more insulin resistance biomarkers in the sample to insulin resistance reference levels of the one or more biomarkers in order to determine the subject's insulin resistance score. The method may employ any number of markers selected from those listed in Table 4, including 1, 2, 3, 4, 5, 6, 7, 8, 9, 10, or more markers. Multiple biomarkers may be correlated with a given condition, such as being insulin resistant, by any method, including statistical methods such as regression analysis.

Any suitable method may be used to analyze the biological sample in order to determine the level(s) of the one or more biomarkers in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

After the level(s) of the one or more biomarker(s) is determined, the level(s) may be compared to disease or condition reference level(s) or reference curves of the one or more biomarker(s) to determine a rating for each of the one or more biomarker(s) in the sample. The rating(s) may be aggregated using any algorithm to create a score, for example, an insulin resistance (IR) score, for the subject. The algorithm may take into account any factors relating to the disease or condition, such as being insulin resistant, including the number of biomarkers, the correlation of the biomarkers to the disease or condition, etc.

In one example, the subject's predicted insulin resistance level may be used to determine the probability that the subject is insulin resistant (i.e. determine the subject's IR Score). For example, using a standardized curve generated using one or more biomarkers listed in Table 4, a subject predicted to have an insulin resistance level of 9, may have a 10% probability of being insulin resistant. Alternatively, in another example, a subject predicted to have an insulin resistance level of 3 may have a 90% probability of being insulin resistant.

### III. Monitoring Disease or Condition Progression/Regression

The identification of biomarkers herein allows for monitoring progression/regression of insulin resistance or related conditions in a subject. A method of monitoring the progression/regression insulin resistance or related condition in a subject comprises (1) analyzing a first biological sample from a subject to determine the level(s) of one or more biomarkers for insulin resistance listed in Table 4, and combinations thereof, in the first sample obtained from the subject at a first time point, (2) analyzing a second biological sample from a subject to determine the level(s) of the one or more biomarkers, the second sample obtained from the subject at a second time point, and (3) comparing the level(s) of one or more biomarkers in the first sample to the level(s) of the one or more biomarkers in the second sample in order to monitor the progression/regression of the disease or condition in the subject. The results of the method are indicative of the course of insulin resistance (i.e., progression or regression, if any change) in the subject.

In one embodiment, the results of the method may be based on an Insulin Resistance (IR) Score which is representative of the probability of insulin resistance in the subject and which can be monitored over time. By comparing the IR Score from a first time point sample to the IR Score from at least a second time point sample the progression or regression of IR can be determined. Such a method of monitoring the progression/regression of insulin resistance, pre-diabetes and/or type-2 diabetes in a subject comprises (1) analyzing a first biological sample from a subject to determine an IR score for the first sample obtained from the subject at a first time point, (2) analyzing a second biological sample from a subject to determine a second IR score, the second sample obtained from the subject at a second time point, and (3) comparing the IR score in the first sample to the IR score in the second sample in order to monitor the progression/regression of insulin resistance, pre-diabetes and/or type-2 diabetes in the subject. An increase in the probability of insulin resistance from the first to the second time point is indicative of the progression of insulin resistance in the subject, while a decrease in the probability from the first to the second time points is indicative of the regression of insulin resistance in the subject.

Using the biomarkers and algorithm of the instant invention for progression monitoring may guide, or assist a physician's decision to implement preventative measures such as dietary restrictions, exercise, and/or early-stage drug treatment.

### IV. Determining Predisposition to a Disease or Condition

The biomarkers identified herein may also be used in the determination of whether a subject not exhibiting any symptoms of a disease or condition, such as insulin resistance or an insulin resistance-related condition such as, for example, myocardial infarction, myocardial ischemia, coronary artery disease, nephropathy, chronic kidney disease, hypertension, impaired glucose tolerance, atherosclerosis, dyslipidemia, or dysglycemia, is predisposed to developing such a condition. The biomarkers may be used, for example, to determine whether a subject is predisposed to developing or becoming, for example, insulin resistant. Such methods of determining whether a subject having no symptoms of a particular disease or condition such as impaired insulin resistance, being insulin resistant, or having an insulin resistance-related condition, is predisposed to developing a particular disease or condition comprise (1) analyzing a biological sample from a subject to determine the level(s) of one or more biomarkers listed in Table 4 in the sample and (2) comparing the level(s) of the one or more biomarkers in the sample to disease- or condition-positive and/or disease- or condition-negative reference levels of the one or more biomarkers in order to determine whether the subject is predisposed to developing the respective disease or condition. For example, the identification of biomarkers for insulin resistance allows for the determination of whether a subject having no symptoms of insulin resistance is predisposed to developing insulin resistance. A method of determining whether a subject having no symptoms of insulin resistance is predisposed to becoming insulin resistant comprises (1) analyzing a biological sample from a subject to determine the level(s) of one or more biomarkers listed Table 4 in the sample and (2) comparing the level(s) of the one or more biomarkers in the sample to insulin resistance-positive and/or insulin resistance-negative reference levels of the one or more biomarkers in order to determine whether the subject is predisposed to developing insulin resistance. The results of the method may be used along with other methods (or the results thereof) useful in the clinical determination of whether a subject is predisposed to developing the disease or condition.

After the level(s) of the one or more biomarkers in the sample are determined, the level(s) are compared to disease- or condition-positive and/or disease- or condition-negative reference levels in order to predict whether the subject is predisposed to developing a disease or condition such as insulin resistance, pre-diabetes, or type-2 diabetes. Levels of the one or more biomarkers in a sample corresponding to the disease- or condition-positive reference levels (e.g., levels that are the same as the reference levels, substantially the same as the reference levels, above and/or below the minimum and/or maximum of the reference levels, and/or within the range of the reference levels) are indicative of the subject being predisposed to developing the disease or condition. Levels of the one or more biomarkers in a sample corresponding to disease- or condition-negative reference levels (e.g., levels that are the same as the reference levels, substantially the same as the reference levels, above and/or below the minimum and/or maximum of the reference levels, and/or within the range of the reference levels) are indicative of the subject not being predisposed to developing the disease or condition. In addition, levels of the one or more biomarkers that are differentially present (especially at a level that is statistically significant) in the sample as compared to disease- or condition-negative reference levels may be indicative of the subject being predisposed to developing the disease or condition. Levels of the one or more biomarkers that are differentially present (especially at a level that is statistically significant) in the sample as compared to disease-condition-positive reference levels are indicative of the subject not being predisposed to developing the disease or condition.

By way of example, after the level(s) of the one or more biomarkers in the sample are determined, the level(s) are compared to insulin resistance-positive and/or insulin resistance-negative reference levels in order to predict whether the subject is predisposed to developing insulin resistance. Levels of the one or more biomarkers in a sample corresponding to the insulin resistance-positive reference levels (e.g., levels that are the same as the reference levels, substantially the same as the reference levels, above and/or below the minimum and/or maximum of the reference levels, and/or within the range of the reference levels) are indicative of the subject being predisposed to developing insulin resistance. Levels of the one or more biomarkers in a sample corresponding to the insulin resistance-negative reference levels (e.g., levels that are the same as the reference levels, substantially the same as the reference levels, above and/or below the minimum and/or maximum of the reference levels, and/or within the range of the reference levels) are indicative of the subject not being predisposed to developing insulin resistance. In addition, levels of the one or more biomarkers that are differentially present (especially at a level that is statistically significant) in the sample as compared to insulin resistance-negative reference levels are indicative of the subject being predisposed to developing insulin resistance. Levels of the one or more biomarkers that are differentially present (especially at a level that is statistically significant) in the sample as compared to insulin resistance-positive reference levels are indicative of the subject not being predisposed to developing insulin resistance.

Furthermore, it may also be possible to determine reference levels specific to assessing whether or not a subject that does not have a disease or condition such as insulin resistance, pre-diabetes, or type-2 diabetes, is predisposed to developing a disease or condition. For example, it may be possible to determine reference levels of the biomarkers for assessing different degrees of risk (e.g., low, medium, high) in a subject for developing a disease or condition. Such reference levels could be used for comparison to the levels of the one or more biomarkers in a biological sample from a subject.

Example 13 illustrates the prediction, based on measurement of certain biomarkers, of whether a subject will progress to having impaired glucose tolerance, or dyslipidemia.

### V. Monitoring Therapeutic Efficacy:

The biomarkers provided also allow for the assessment of the efficacy of a composition for treating a disease or condition such as insulin resistance, pre-diabetes, or type-2 diabetes. For example, the identification of biomarkers for insulin resistance also allows for assessment of the efficacy of a composition for treating insulin resistance as well as the assessment of the relative efficacy of two or more compositions for treating insulin resistance. Such assessments may be used, for example, in efficacy studies as well as in lead selection of compositions for treating the disease or condition. In addition, such assessments may be used to monitor the efficacy of surgical procedures and/or lifestyle interventions on insulin resistance in a subject. Surgical procedures include bariatric surgery, while lifestyle interventions include diet modification or reduction, exercise programs, and the like.

Thus, in one such embodiment, provided are methods of assessing the efficacy of a composition for treating a disease or condition such as insulin resistance, or related condition comprising (1) analyzing, from a subject (or group of subjects) having a disease or condition such as insulin resistance, or related condition and currently or previously being treated with a composition, a biological sample (or group of samples) to determine the level(s) of one or more biomarkers for insulin resistance selected from the biomarkers listed in Table 4, and (2) comparing the level(s) of the one or more biomarkers in the sample to (a) level(s) of the one or more biomarkers in a previously-taken biological sample from the subject, wherein the previously-taken biological sample was obtained from the subject before being treated with the composition, (b) disease- or condition-positive reference levels of the one or more biomarkers, (c) disease- or condition-negative reference levels of the one or more biomarkers, (d) disease- or condition-progression-positive reference levels of the one or more biomarkers, and/or (e) disease- or condition-regression-positive reference levels of the one or more biomarkers. The results of the comparison are indicative of the efficacy of the composition for treating the respective disease or condition.

In another embodiment, methods of assessing the efficacy of a surgical procedure for treating a disease or condition such as insulin resistance, or related condition comprising (1) analyzing, from a subject (or group of subjects) having insulin resistance, or related condition, and having previously undergone a surgical procedure, a biological sample (or group of samples) to determine the level(s) of one or more biomarkers for insulin resistance selected from the biomarkers listed in Table 4, and (2) comparing the level(s) of the one or more biomarkers in the sample to (a) level(s) of the one or more biomarkers in a previously-taken biological sample from the subject, wherein the previously-taken biological sample was obtained from the subject before undergoing the surgical procedure or taken immediately after undergoing the surgical procedure, (b) insulin resistance-positive reference levels of the one or more biomarkers, (c) insulin resistance-negative reference levels of the one or more biomarkers, (d) insulin resistance-progression-positive reference levels of the one or more biomarkers, and/or (e) insulin resistance-regression-positive reference levels of the one or more biomarkers. The results of the comparison are indicative of the efficacy of the surgical procedure for treating the respective disease or condition. In one embodiment, the surgical procedure is a gastro-intestinal surgical procedure, such as bariatric surgery.

The change (if any) in the level(s) of the one or more biomarkers over time may be indicative of progression or regression of the disease or condition in the subject. To characterize the course of a given disease or condition in the subject, the level(s) of the one or more biomarkers in the first sample, the level(s) of the one or more biomarkers in the second sample, and/or the results of the comparison of the levels of the biomarkers in the first and second samples may be compared to the respective disease- or condition-positive and/or disease- or condition-negative reference levels of the one or more biomarkers. If the comparisons indicate that the level(s) of the one or more biomarkers are increasing or decreasing over time (e.g., in the second sample as compared to the first sample) to become more similar to the disease- or condition-positive reference levels (or less similar to the disease- or condition-negative reference levels), then the results are indicative of the disease's or condition's progression. If the comparisons indicate that the level(s) of the one or more biomarkers are increasing or decreasing over time to become more similar to the disease- or condition-negative reference levels (or less similar to the disease- or condition-positive reference levels), then the results are indicative of the disease's or condition's regression.

For example, in order to characterize the course of insulin resistance in the subject, the level(s) of the one or more biomarkers in the first sample, the level(s) of the one or more biomarkers in the second sample, and/or the results of the comparison of the levels of the biomarkers in the first and second samples may be compared to insulin resistance-positive and/or insulin resistance-negative reference levels of the one or more biomarkers. If the comparisons indicate that the level(s) of the one or more biomarkers are increasing or decreasing over time (e.g., in the second sample as compared to the first sample) to become more similar to the insulin resistance-positive reference levels (or less similar to the insulin resistance-negative reference levels), then the results are indicative of insulin resistance progression. If the comparisons indicate that the level(s) of the one or more biomarkers are increasing or decreasing over time to become more similar to the insulin resistance-negative reference levels (or less similar to the insulin resistance-positive reference levels), then the results are indicative of insulin resistance regression.

The second sample may be obtained from the subject any period of time after the first sample is obtained. In one aspect, the second sample is obtained 1, 2, 3, 4, 5, 6, or more days after the first sample or after the initiation of the administration of a composition, surgical procedure, or lifestyle intervention. In another aspect, the second sample is obtained 1, 2, 3, 4, 5, 6, 7, 8, 9, 10, or more weeks after the first sample or after the initiation of the administration of a composition, surgical procedure, or lifestyle intervention. In another aspect, the second sample may be obtained 1, 2, 3, 4, 5, 6, 7, 8, 9, 10, 11, 12, or more months after the first sample or after the initiation of the administration of a composition, surgical procedure, or lifestyle intervention.

The course of a disease or condition such as being insulin resistant, or pre-diabetic, type-2 diabetic in a subject may also be characterized by comparing the level(s) of the one or more biomarkers in the first sample, the level(s) of the one or more biomarkers in the second sample, and/or the results of the comparison of the levels of the biomarkers in the first and second samples to disease- or condition-progression-positive and/or disease- or condition-regression-positive reference levels. If the comparisons indicate that the level(s) of the one or more biomarkers are increasing or decreasing over time (e.g., in the second sample as compared to the first sample) to become more similar to the disease- or condition-progression-positive reference levels (or less similar to the disease- or condition-regression-positive reference levels), then the results are indicative of the disease or condition progression. If the comparisons indicate that the level(s) of the one or more biomarkers are increasing or decreasing over time to become more similar to the disease- or condition-regression-positive reference levels (or less similar to the disease- or condition-progression-positive reference levels), then the results are indicative of disease or condition regression.

As with the other methods described herein, the comparisons made in the methods of monitoring progression/regression of a disease or condition such as being insulin resistant, pre-diabetic, or type-2 diabetic in a subject may be carried out using various techniques, including simple comparisons, one or more statistical analyses, and combinations thereof.

The results of the method may be used along with other methods (or the results thereof) useful in the clinical monitoring of progression/regression of the disease or condition in a subject.

As described above in connection with methods of diagnosing (or aiding in the diagnosis of) a disease or condition such as being insulin resistant, pre-diabetic, or type-2 diabetic, any suitable method may be used to analyze the biological samples in order to determine the level(s) of the one or more biomarkers in the samples. In addition, the level(s) one or more biomarkers, including a combination of all of the biomarkers in Table 4 or any fraction thereof, may be determined and used in methods of monitoring progression/regression of the respective disease or condition in a subject.

Such methods could be conducted to monitor the course of disease or condition development in subjects, for example the course of pre-diabetes to type-2 diabetes in a subject having pre-diabetes, or could be used in subjects not having a disease or condition (e.g., subjects suspected of being predisposed to developing the disease or condition) in order to monitor levels of predisposition to the disease or condition.

Clinical studies from around the world have been carried out to test whether anti-diabetic therapies, such as metformin or acarbose, can prevent diabetes progression in pre-diabetic patients. These studies have shown that such therapies can prevent diabetes onset. From the U.S. Diabetes Prevention Program (DPP), metformin reduced the rate of progression to diabetes by 38% and lifestyle and exercise intervention reduced the rate of progression to diabetes by 56%. Because of such successes, the ADA has revised its 2008 Standards of Medical Care in Diabetes to include the following statements in the section on Prevention/Delay of Type 2 Diabetes: “In addition to lifestyle counseling, metformin may be considered in those who are at very high risk (combined IFG and IGT plus other risk factors) and who are obese and under 60 years of age.”

Pharmaceutical companies have carried out studies to assess whether certain classes of drugs, such as the PPARγ class of insulin sensitizers (e.g. muraglitozar), can prevent diabetes progression. Similar to the DPP trial, some of these studies have shown great promise and success for preventing diabetes, whereas others have exposed a certain amount of risk associated with certain anti-diabetic pharmacologic treatments when given to the general pre-diabetic population as defined by current IR diagnostics. Pharmaceutical companies are in need of diagnostics that can identify and stratify high risk pre-diabetics so they can assess the efficacy of their pre-diabetic therapeutic candidates more effectively and safely. In some embodiments, subjects that are identified as more insulin resistant may be more likely to respond to an insulin sensitizer composition.

Considering the infrequency of the oral glucose tolerance test (OGTT) procedures in the clinical setting, a new diagnostic test that directly measures insulin resistance in a fasted sample would enable a physician to identify and stratify patients who are moving toward the etiology of pre-diabetes and type-2 diabetes much earlier.

### VI. Identification of Responders and Non-Responders to Therapeutic:

The biomarkers provided also allow for the identification of subjects in whom the composition for treating a disease or condition such as insulin resistance, pre-diabetes, or type-2 diabetes is efficacious (i.e. patient responds to therapeutic). For example, the identification of biomarkers for insulin resistance also allows for assessment of the subject's response to a composition for treating insulin resistance as well as the assessment of the relative patient response to two or more compositions for treating insulin resistance. Such assessments may be used, for example, in selection of compositions for treating the disease or condition for certain subjects, or in the selection of subjects into a course of treatment or clinical trial.

Thus, also provided are methods of predicting the response of a patient to a composition for treating a disease or condition such as insulin resistance, pre-diabetes, or type-2 diabetes comprising (1) analyzing, from a subject (or group of subjects) having a disease or condition such as insulin resistance, pre-diabetes, or type-2 diabetes and currently or previously being treated with a composition, a biological sample (or group of samples) to determine the level(s) of one or more biomarkers for insulin resistance selected from the biomarkers listed in Table 4 and (2) comparing the level(s) of the one or more biomarkers in the sample to (a) level(s) of the one or more biomarkers in a previously-taken biological sample from the subject, wherein the previously-taken biological sample was obtained from the subject before being treated with the composition, (b) disease- or condition-positive reference levels of the one or more biomarkers, (c) disease- or condition-negative reference levels of the one or more biomarkers, (d) disease- or condition-progression-positive reference levels of the one or more biomarkers, and/or (e) disease- or condition-regression-positive reference levels of the one or more biomarkers. The results of the comparison are indicative of the response of the patient to the composition for treating the respective disease or condition.

The change (if any) in the level(s) of the one or more biomarkers over time may be indicative of response of the subject to the therapeutic. To characterize the course of a given therapeutic in the subject, the level(s) of the one or more biomarkers in the first sample, the level(s) of the one or more biomarkers in the second sample, and/or the results of the comparison of the levels of the biomarkers in the first and second samples may be compared to the respective disease- or condition-positive and/or disease- or condition-negative reference levels of the one or more biomarkers. If the comparisons indicate that the level(s) of the one or more biomarkers are increasing or decreasing over time (e.g., in the second sample as compared to the first sample) to become more similar to the disease- or condition-positive reference levels (or less similar to the disease- or condition-negative reference levels), then the results are indicative of the patient not responding to the therapeutic. If the comparisons indicate that the level(s) of the one or more biomarkers are increasing or decreasing over time to become more similar to the disease- or condition-negative reference levels (or less similar to the disease- or condition-positive reference levels), then the results are indicative of the patient responding to the therapeutic.

For example, in order to characterize the patient response to a therapeutic for insulin resistance, the level(s) of the one or more biomarkers in the first sample, the level(s) of the one or more biomarkers in the second sample, and/or the results of the comparison of the levels of the biomarkers in the first and second samples may be compared to insulin resistance-positive and/or insulin resistance-negative reference levels of the one or more biomarkers. If the comparisons indicate that the level(s) of the one or more biomarkers are increasing or decreasing over time (e.g., in the second sample as compared to the first sample) to become more similar to the insulin resistance-positive reference levels (or less similar to the insulin resistance-negative reference levels), then the results are indicative of non-response to the therapeutic. If the comparisons indicate that the level(s) of the one or more biomarkers are increasing or decreasing over time to become more similar to the insulin resistance-negative reference levels (or less similar to the insulin resistance-positive reference levels), then the results are indicative of response to the therapeutic.

The second sample may be obtained from the subject any period of time after the first sample is obtained. In one aspect, the second sample is obtained 1, 2, 3, 4, 5, 6, or more days after the first sample. In another aspect, the second sample is obtained 1, 2, 3, 4, 5, 6, 7, 8, 9, 10, or more weeks after the first sample or after the initiation of treatment with the composition. In another aspect, the second sample may be obtained 1, 2, 3, 4, 5, 6, 7, 8, 9, 10, 11, 12, or more months after the first sample or after the initiation of treatment with the composition.

As with the other methods described herein, the comparisons made in the methods of determining a patient response to a therapeutic for a disease or condition such as insulin resistance, pre-diabetes, or type-2 diabetes in a subject may be carried out using various techniques, including simple comparisons, one or more statistical analyses, and combinations thereof.

The results of the method may be used along with other methods (or the results thereof) useful in determining a patient response to a therapeutic for the disease or condition in a subject.

As described above in connection with methods of diagnosing (or aiding in the diagnosis of) a disease or condition such as insulin resistance, pre-diabetes, or type-2 diabetes, any suitable method may be used to analyze the biological samples in order to determine the level(s) of the one or more biomarkers in the samples. In addition, the level(s) one or more biomarkers, including a combination of all of the biomarkers in Table 4, or any fraction thereof, may be determined and used in methods of monitoring progression/regression of the respective disease or condition in a subject.

Such methods could be conducted to monitor the patient response to a therapeutic for a disease or condition development in subjects, for example the course of pre-diabetes to type-2 diabetes in a subject having pre-diabetes, or could be used in subjects not having a disease or condition (e.g., subjects suspected of being predisposed to developing the disease or condition) in order to monitor levels of predisposition to the disease or condition.

Pharmaceutical companies have carried out studies to assess whether certain classes of drugs, such as the PPARγ class of insulin sensitizers, can prevent diabetes progression. Some of these studies have shown great promise and success for preventing diabetes, whereas others have exposed a certain amount of risk associated with certain anti-diabetic pharmacologic treatments when given to the general pre-diabetic population as defined by current IR diagnostics. Pharmaceutical companies are in need of diagnostics that can identify responders and non-responders in order to stratify high risk pre-diabetics to assess the efficacy of their pre-diabetic therapeutic candidates more effectively and safely. A new diagnostic test that discriminates non-responding from responding patients to a therapeutic would enable pharmaceutical companies to identify and stratify patients that are likely to respond to the therapeutic agent and target specific therapeutics for certain cohorts that are likely to respond to the therapeutic.

### VII. Methods of Screening a Composition for Activity in Modulating Biomarkers

The biomarkers provided herein also allow for the screening of compositions for activity in modulating biomarkers associated with a disease or condition, such as insulin resistance, pre-diabetes, type-2 diabetes, which may be useful in treating the disease or condition. Such methods comprise assaying test compounds for activity in modulating the levels of one or more biomarkers selected from the respective biomarkers listed in the respective tables. Such screening assays may be conducted in vitro and/or in vivo, and may be in any form known in the art useful for assaying modulation of such biomarkers in the presence of a test composition such as, for example, cell culture assays, organ culture assays, and in vivo assays (e.g., assays involving animal models). For example, the identification of biomarkers for insulin resistance also allows for the screening of compositions for activity in modulating biomarkers associated with insulin resistance, which may be useful in treating insulin resistance. Methods of screening compositions useful for treatment of insulin resistance comprise assaying test compositions for activity in modulating the levels of one or more biomarkers in Table 4. Although insulin resistance is discussed in this example, the other diseases and conditions such as pre-diabetes and type-2 diabetes may also be diagnosed or aided to be diagnosed in accordance with this method by using one or more of the respective biomarkers as set forth above.

The methods for screening a composition for activity in modulating one or more biomarkers of a disease or condition such as insulin resistance, or related disorder comprise (1) contacting one or more cells with a composition, (2) analyzing at least a portion of the one or more cells or a biological sample associated with the cells to determine the level(s) of one or more biomarkers of a disease or condition selected from the biomarkers provided in Table 4; and (3) comparing the level(s) of the one or more biomarkers with predetermined standard levels for the one or more biomarkers to determine whether the composition modulated the level(s) of the one or more biomarkers. In one embodiment, a method for screening a composition for activity in modulating one or more biomarkers of insulin resistance comprises (1) contacting one or more cells with a composition, (2) analyzing at least a portion of the one or more cells or a biological sample associated with the cells to determine the level(s) of one or more biomarkers of insulin resistance selected from the biomarkers listed in Table 4; and (3) comparing the level(s) of the one or more biomarkers with predetermined standard levels for the one or more biomarkers to determine whether the composition modulated the level(s) of the one or more biomarkers. As discussed above, the cells may be contacted with the composition in vitro and/or in vivo. The predetermined standard levels for the one or more biomarkers may be the levels of the one or more biomarkers in the one or more cells in the absence of the composition. The predetermined standard levels for the one or more biomarkers may also be the level(s) of the one or more biomarkers in control cells not contacted with the composition.

In addition, the methods may further comprise analyzing at least a portion of the one or more cells or a biological sample associated with the cells to determine the level(s) of one or more non-biomarker compounds of a disease or condition, such as insulin resistance, pre-diabetes, and type-2 diabetes. The levels of the non-biomarker compounds may then be compared to predetermined standard levels of the one or more non-biomarker compounds.

Any suitable method may be used to analyze at least a portion of the one or more cells or a biological sample associated with the cells in order to determine the level(s) of the one or more biomarkers (or levels of non-biomarker compounds). Suitable methods include chromatography (e.g., HPLC, gas chromatography, liquid chromatography), mass spectrometry (e.g., MS, MS-MS), ELISA, antibody linkage, other immunochemical techniques, biochemical or enzymatic reactions or assays, and combinations thereof. Further, the level(s) of the one or more biomarkers (or levels of non-biomarker compounds) may be measured indirectly, for example, by using an assay that measures the level of a compound (or compounds) that correlates with the level of the biomarker(s) (or non-biomarker compounds) that are desired to be measured.

### VIII. Method of Identifying Potential Drug Targets

The disclosure also provides methods of identifying potential drug targets for diseases or conditions such as insulin resistance, and related conditions, using the biomarkers listed in Table 4. A method for identifying a potential drug target for a disease or condition such as insulin resistance, or a related condition, comprises (1) identifying one or more biochemical pathways associated with one or more biomarkers for insulin resistance selected from the biomarkers listed in Table 4; and (2) identifying an agent (e.g., an enzyme, co-factor, etc.) affecting at least one of the one or more identified biochemical pathways, the agent being a potential drug target for the insulin resistance. For example, the identification of biomarkers for insulin resistance also allows for the identification of potential drug targets for insulin resistance. A method for identifying a potential drug target for insulin resistance comprises (1) identifying one or more biochemical pathways associated with one or more biomarkers for insulin resistance selected from in Table 4, and (2) identifying a protein (e.g., an enzyme) affecting at least one of the one or more identified biochemical pathways, the protein being a potential drug target for insulin resistance. Although insulin resistance is discussed in this example, potential drug target for the other diseases or conditions such as pre-diabetes and type-2 diabetes, may also be identified in accordance with this method by using one or more of the respective biomarkers as set forth above.

In another embodiment, a method of identifying an agent capable of modulating the level of a biomarker of insulin resistance, the method comprising: analyzing a biological sample from a subject at a first time point to determine the level(s) of one or more biomarkers listed in Table 4, contacting the biological sample with a test agent, analyzing the biological sample at a second time point to determine the level(s) of the one or more biomarkers, the second time point being a time after contacting with the test agent, and comparing the level(s) of one or more biomarkers in the sample at the first time point to the level(s) of the one or more biomarkers in the sample at the second time point to identify an agent capable of modulating the level of the one or more biomarkers.

Test agents for use in such methods include any agent capable of modulating the level of a biomarker in a sample. Such agents include, but are not limited to small molecules, nucleic acids, polypeptides, antibodies, and combinations thereof. Nucleic acid agents include antisense nucleic acids, double-stranded RNA, interfering RNA, ribozymes, and the like. In addition, the test agent can target any component in the pathway affecting the biomarker of the present invention or pathways that include such biomarkers.

In one embodiment, biochemical pathways associated with one or more biomarkers listed in Table 4 include pathways involved in the formation of such biomarkers, pathways involved in the degradation of such biomarkers, and/or pathways in which the biomarkers are involved. For example, one biomarker listed in Table 4. Potential targets for insulin resistance therapeutics may thus be identified from any of the enzymes, cofactors, genes, or the like involved in 2-hydroxybutyrate formation, metabolism, or utilization. For example, potential targets in the 2-hydroxybutyrate formation pathway include, lactate dehydrogenase, hydroxybutyric acid dehydrogenase, alanine transaminase, gamma-cystathionase, branched-chain alpha-keto acid dehydrogenase, and the like. Such potential targets can be targeted for any modification of expression, such as increases or decreases of expression. The substrates and enzymes in this pathway and related pathways may be candidates for therapeutic intervention and drug targets. For example, with regard to targeting 2-hydroxybutyrate, inhibition of lactate dehydrogenase (LDH) or activation of hydroxybutyric acid dehydrogenase (HBDH) or branched chain alpha-keto acid dehydrogenase (BCKDH) may be useful as therapeutic treatments of insulin resistance. In another embodiment, a pathway in which 2-hydroxybutyrate is involved is the citrate pathway (TCA pathway). When flux into the TCA cycle is reduced, there is typically an overflow of 2-hydroxybutyrate. Thus, any of the enzymes, co-factors, genes, and the like involved in the TCA cycle may also be targets for potential therapeutic discovery for agents capable of modulating the levels of the biomarkers, or for treating insulin resistance and related disorders.

In addition, metabolites and pathways related to the biomarkers listed in Table 4 may be useful as targets for therapeutic screening. For example, metabolites and pathways related to 2-hydroxybutyrate may also be targets for insulin resistance therapeutics, such as alpha-ketoacids, 3-methyl-2-oxobutyrate and 3-methyl-2-oxovalerate. Furthermore, other metabolites and agents involved in branched chain alpha-keto acid biosynthesis, metabolism, and utilization may also be useful as targets for therapeutic discovery for the treatment of insulin resistance or related conditions.

Another method for identifying a potential drug target for a disease or condition such as insulin resistance, pre-diabetes, and type-2 diabetes comprises (1) identifying one or more biochemical pathways associated with one or more biomarkers for insulin resistance selected from the biomarkers listed Table 4 and one or more non-biomarker compounds of insulin resistance and (2) identifying a protein affecting at least one of the one or more identified biochemical pathways, the protein being a potential drug target for the disease or condition. For example, a method for identifying a potential drug target for insulin resistance comprises (1) identifying one or more biochemical pathways associated with one or more biomarkers for insulin resistance selected from Table 4, and one or more non-biomarker compounds of insulin resistance and (2) identifying a protein affecting at least one of the one or more identified biochemical pathways, the protein being a potential drug target for insulin resistance.

One or more biochemical pathways (e.g., biosynthetic and/or metabolic (catabolic) pathway) are identified that are associated with one or more biomarkers (or non-biomarker compounds). After the biochemical pathways are identified, one or more proteins affecting at least one of the pathways are identified. Preferably, those proteins affecting more than one of the pathways are identified.

A build-up of one metabolite (e.g., a pathway intermediate) may indicate the presence of a ‘block’ downstream of the metabolite and the block may result in a low/absent level of a downstream metabolite (e.g. product of a biosynthetic pathway). In a similar manner, the absence of a metabolite could indicate the presence of a ‘block’ in the pathway upstream of the metabolite resulting from inactive or non-functional enzyme(s) or from unavailability of biochemical intermediates that are required substrates to produce the product. Alternatively, an increase in the level of a metabolite could indicate a genetic mutation that produces an aberrant protein which results in the over-production and/or accumulation of a metabolite which then leads to an alteration of other related biochemical pathways and result in dysregulation of the normal flux through the pathway; further, the build-up of the biochemical intermediate metabolite may be toxic or may compromise the production of a necessary intermediate for a related pathway. It is possible that the relationship between pathways is currently unknown and this data could reveal such a relationship.

The proteins identified as potential drug targets may then be used to identify compositions that may be potential candidates for treating a particular disease or condition, such as insulin resistance, including compositions for gene therapy.

### IX. Methods of Treatment

In another aspect, methods for treating a disease or condition such as insulin resistance, pre-diabetes, and type-2 diabetes are provided. The methods generally involve treating a subject having a disease or condition such as insulin resistance. pre-diabetes, and type-2 diabetes with an effective amount of one or more biomarker(s) that are lowered in a subject having the disease or condition as compared to a healthy subject not having the disease or condition. The biomarkers that may be administered may comprise one or more of the biomarkers Table 4 that are decreased in a disease or condition state as compared to subjects not having that disease or condition. Such biomarkers could be isolated based on the identity of the biomarker compound (i.e. compound name). Although insulin resistance is discussed in this example, the other diseases or conditions, such as pre-diabetes and type-2 diabetes, may also be treated in accordance with this method by using one or more of the respective biomarkers as set forth above.

### X. Methods of Using the Biomarkers for Other Diseases or Conditions

In another aspect, at least some of the biomarkers disclosed herein for a particular disease or condition may also be biomarkers for other diseases or conditions. For example, it is believed that at least some of the insulin resistance biomarkers may be used in the methods described herein for other diseases or conditions (e.g., metabolic syndrome, polycystic ovary syndrome (PCOS), hypertension, cardiovascular disease, non-alcoholic steatohepatitis (NASH)). That is, the methods described herein with respect to insulin resistance may also be used for diagnosing (or aiding in the diagnosis of) a disease or condition such as type-2 diabetes, metabolic syndrome, atherosclerosis, coronary artery disease, cardiomyopathy, PCOS, NASH, myocardial infarction, myocardial ischemia, nephropathy, chronic kidney disease, (ckd) or hypertension, methods of monitoring progression/regression of such a disease or condition, methods of assessing efficacy of compositions for treating such a disease or condition, methods of screening a composition for activity in modulating biomarkers associated with such a disease or condition, methods of identifying potential drug targets for such diseases and conditions, and methods of treating such diseases and conditions. Such methods could be conducted as described herein with respect to insulin resistance.

### XI. Other Methods

Other methods of using the biomarkers discussed herein are also contemplated. For example, the methods described in U.S. Pat. Nos. 7,005,255; 7,329,489; 7,550,258; 7,550,260; 7,553,616; 7,635,556; 7,682,782; and 7,682,784 may be conducted using a small molecule profile comprising one or more of the biomarkers disclosed herein.

### EXAMPLES

### I. General Methods

A. Identification of Metabolic Profiles

Each sample was analyzed to determine the concentration of several hundred metabolites. Analytical techniques such as GC-MS (gas chromatography-mass spectrometry) and LC-MS (liquid chromatography-mass spectrometry) were used to analyze the metabolites. Multiple aliquots were simultaneously, and in parallel, analyzed, and, after appropriate quality control (QC), the information derived from each analysis was recombined. Every sample was characterized according to several thousand characteristics, which ultimately amount to several hundred chemical species. The techniques used were able to identify novel and chemically unnamed compounds.

B. Statistical Analysis:

The data was analyzed using several statistical methods to identify molecules (either known, named metabolites or unnamed metabolites) present at differential levels in a definable population or subpopulation (e.g., biomarkers for insulin resistant biological samples compared to control biological samples or compared to insulin sensitive patients) useful for distinguishing between the definable populations (e.g., insulin resistance and control, insulin resistance and insulin sensitive, insulin resistance and type-2 diabetes). Other molecules (either known, named metabolites or unnamed metabolites) in the definable population or subpopulation were also identified.

Random forest analyses were used for classification of samples into groups (e.g. disease or healthy, insulin resistant or normal insulin sensitivity). Random forests give an estimate of how well we can classify individuals in a new data set into each group, in contrast to a t-test, which tests whether the unknown means for two populations are different or not. Random forests create a set of classification trees based on continual sampling of the experimental units and compounds. Then each observation is classified based on the majority votes from all the classification trees.

Regression analysis was performed using the Random Forest Regression method and the Univariate Correlation/Linear Regression method to build models that are useful to identify the biomarker compounds that are associated with disease or disease indicators (e.g. Rd) and then to identify biomarker compounds useful to classify individuals according to for example, the level of glucose utilization as normal, insulin impaired, or insulin resistant. Biomarker compounds that are useful to predict disease or measures of disease (e.g. Rd) and that are positively or negatively correlated with disease or measures of disease (e.g. Rd) were identified in these analyses. All of the biomarker compounds identified in these analyses were statistically significant (p<0.05, q<0.1).

Recursive partitioning relates a ‘dependent’ variable (Y) to a collection of independent ('predictor') variables (X) in order to uncover—or simply understand—the elusive relationship, Y=f(X). The analysis was performed with the JMP program (SAS) to generate a decision tree. The statistical significance of the “split” of the data can be placed on a more quantitative footing by computing p-values, which discern the quality of a split relative to a random event. The significance level of each “split” of data into the nodes or branches of the tree was computed as p-values, which discern the quality of the split relative to a random event. It was given as LogWorth, which is the negative log 10 of a raw p-value.

Statistical analyses were performed with the program “R” available on the worldwide web at the website cran.r-project.org and in JMP 6.0.2 (SAS® Institute, Cary, N.C.).

### Example 2

### Biomarkers of Insulin Resistance

2A: Identification of Biomarkers that Correlate with Insulin Resistance

Biomarkers were discovered that correlate with the glucose disposal rate (i.e. Rd), a measure of insulin resistance. An initial panel of biomarkers was then narrowed for the development of targeted assays (to determine the level of the biomarkers form a biological sample). An algorithm to predict insulin resistance in a subject was also developed.

An initial panel of biomarkers that correlate with insulin resistance was developed using several studies. In a first study, plasma samples were collected from 113 lean, obese or diabetic subjects that had received treatment with one of three different thiazolidinedione drugs (T=troglitazone, R=rosiglitazone, or P=pioglitazone) (Table 1). Base line samples obtained from the subjects prior to treatment (S=baseline) served as controls. One to three plasma samples were obtained from each subject, with samples collected at baseline (all subjects; A), and after 12 weeks (B) or 4 weeks (C) of drug treatment (Table 2). Glucose disposal rate was measured in every subject by the hyperinsulinemic euglycemic (HI) clamp following each blood draw. A total of 198 plasma samples were collected for analysis.

TABLE 1
Sex and treatments of the study 1 cohort.

| GROUP | SEX | P | R | S | T | Total |
|---|---|---|---|---|---|---|
| Lean | F | 1 | 0 | 1 | 1 | 3 |
| | M | 7 | 0 | 12 | 8 | 27 |
| Obese | F | 2 | 0 | 3 | 1 | 6 |
| | M | 7 | 0 | 14 | 8 | 29 |
| Diabetic | F | 0 | 7 | 3 | 1 | 11 |
| | M | 8 | 13 | 7 | 9 | 37 |

| | | | | | |
|---|---|---|---|---|---|
| Total | 25 | 20 | 40 | 28 | 113 |

TABLE 2
Treatment and collection time of the study 1 cohort.

| GROUP | TIME | P | R | S | T | Total |
|---|---|---|---|---|---|---|
| Lean | A | 8 | 0 | 13 | 9 | 30 |
| | B | 8 | 0 | 0 | 8 | 16 |
| Obese | A | 9 | 0 | 17 | 9 | 35 |
| | B | 9 | 0 | 0 | 9 | 18 |
| | C | 9 | 0 | 0 | 0 | 9 |
| Diabetic | A | 8 | 19 | 10 | 9 | 46 |
| | B | 8 | 20 | 0 | 10 | 38 |
| | C | 6 | 0 | 0 | 0 | 6 |

| | | | | | |
|---|---|---|---|---|---|
| Total | 65 | 39 | 40 | 54 | 198 |

In a second study, plasma samples were collected from 402 subjects that were balanced for age and sex. The subjects underwent HI clamp to determine the glucose disposal rate (Rd) of each individual. Based upon an Oral Glucose Tolerance Test (OGTT) or a Fasting Plasma Glucose Test (FPGT) the glucose tolerance of the subjects was designated as Normal glucose tolerance (NGT), Impaired Fasting Glucose (IFG) or Impaired Glucose Tolerance (IGT). The cohort is described in Table 3.

TABLE 3
Cohort Description, Study 2

| | | | Age | | Rd | |
|---|---|---|---|---|---|---|
| Group | Sex | N | Mean | Std Dev | Mean | Std Dev |
| NGT | female | 155 | 44.64 | 8.02 | 8.5 | 3.09 |
| | male | 148 | 44.03 | 8.62 | 8.38 | 2.77 |
| IFG | female | 5 | 46.8 | 6.53 | 6.13 | 3.32 |
| | male | 12 | 45.25 | 9.63 | 4.67 | 2.57 |
| IGT | female | 45 | 45.56 | 7.81 | 4.19 | 1.81 |
| | male | 37 | 45.73 | 7.8 | 4.73 | 2.27 |

Abbreviations
Rd: Glucose disposal rate
NGT: Normal Glucose Tolerant (OGTT, <140 mg/dL or <7.8 mmol/L)
IFG: Impaired Fasting Glucose (Fasting plasma glucose, 100-125 mg/dL or 5.6-6.9 mmol/L)
IGT: Impaired Glucose Tolerant (OGTT, 140-199 mg/dL or 7.8-11.0 mmol/L)

All samples from both studies were analyzed by GC-MS and LC-MS to identify and quantify the small molecules present in the samples. Over 400 compounds were detected in the samples.

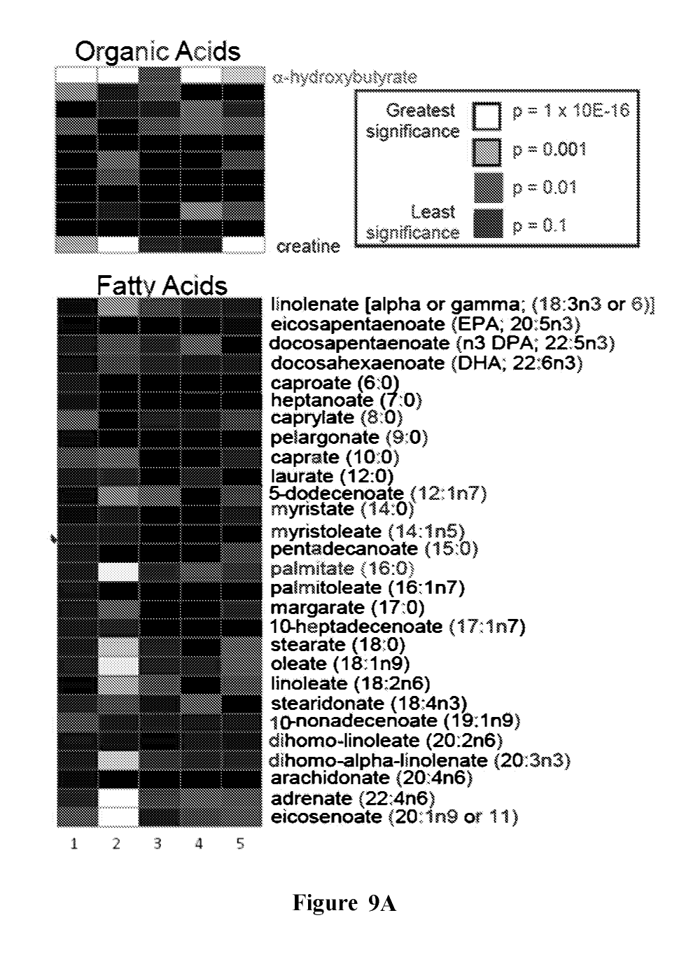

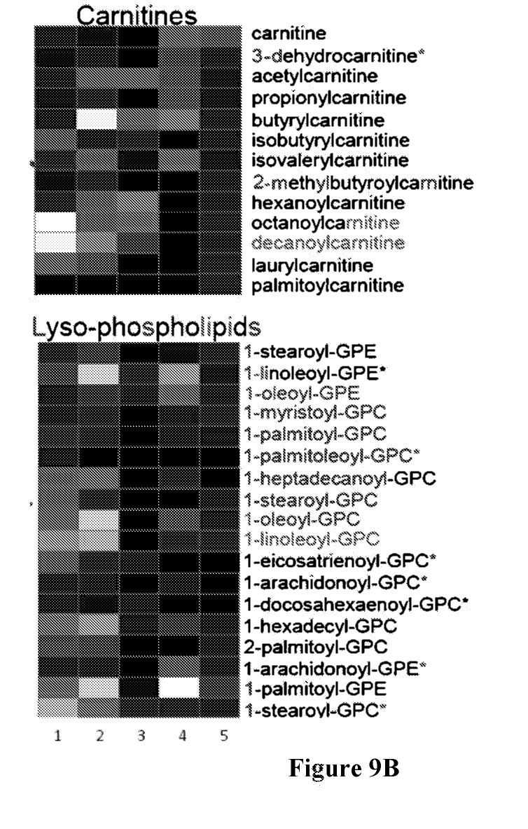

Statistical analyses were performed to determine the compounds that are useful as biomarkers. The biomarkers identified were divided among biochemical pathways and by significance for distinguishing between classes of individuals (i.e., NGT-IS, NGT-IR, IGT, IFG) as illustrated in FIG. 9. FIG. 9 highlights the biochemical profiles obtained for the biomarkers in a heat map graphical representation of p-values obtained from t-test statistical analysis of the global biochemical profiling of metabolites measured in plasma collected from NGT-IS, NGT-IR, IGT, and IFG subjects. Columns 1-5 designate the following comparisons for each listed biomarker: 1, NGT-IS vs. NGT-IR; 2, NGT-IS vs. IGT; 3, NGT-IR vs. IGT; 4, NGT-IS vs. IFG; 5, IGT vs. IFG (white, most statistically significant (p≦1.0E-16); light grey (1.0E-16≦p≦0.001), dark grey (0.001≦p≦0.01), and black, not significant (p≧0.1)). For example, 2-hydroxybutyrate and creatine were significant biomarkers for distinguishing NGT-IS subjects from NGT-IR subjects and NGT-IS subjects from IGT subjects. The fatty acid-related biomarkers (i.e., palmitate, stearate, oleate, heptadecanoate, 10-nonadecanoate, linoleate, dihomolinoleate, stearidonate, docosatetraenoate, docosapentaenoate, docosaheanoate, and margarate) were significant markers for distinguishing NGT-IS subjects from IGT subjects. In addition, the acyl carnitines (i.e., acyl-carnitine, octanoylcarnitine, decanoylcarnitine, laurylcarnitine, carnitine, 3-dehydrocarnitine, acetylcarnitine, propionylcarnitine, butyrylcarnitine, isobutyrylcarnitine, isovalerylcarnitine, hexanoylcarnitine), lysoglycerophospholipids (including both glycerophosphocholines (GPC) and lysoglycerophosphocholines (LPC); i.e., 1-eicosatrienoyl-glycerophosphocholine, 2-palmitoyl-glycerophosphocholine, 1-heptadecanoylglycerophosphocholine, 1-stearoylglycerophosphocholine, 1-oleoylglycerophosphocholine, 1-linoleoylglycerophosphocholine, and 1-hexadecylglycerophosphocholine), and N-acylphophoethanolamines (i.e., 1-palmitoyl-glycerophosphoethanolamine, 1-arachidonoyl-glycerophosphoethanolamine, 1-linoleoyl-glycerophosphoethanolamine, 1-oleoyl-glycerophosphoethanolamine) were significant markers for distinguishing NGT-IS subjects from NGT-IR subjects, NGT-IS subjects from IFG subjects, and NGT-IS subjects from IGT subjects.

Linear regression was used to correlate the baseline levels of individual compounds with the glucose disposal rate (Rd) as measured by the euglycemic hyperinsulinemic clamp for each individual. This analysis was followed by Random Forest analysis to identify variables most useful for Rd modeling. Further, the initial panel of biomarkers was narrowed down for the development of targeted assays for detecting levels of certain biomarkers. As listed below in Table 4, biomarkers were discovered that were correlated with indicators of insulin sensitivity as measured by the HI clamp (i.e., the glucose disposal rate (Rd), Mffm or Mwbm).

TABLE 4
Insulin Resistance Biomarkers

| | | | HMDB |
|---|---|---|---|
| | | | Accession |
| | Common Name | IUPAC from NCBI Pubchem | No.¹ |
| 1 | Creatine | 2-[carbamimidoyl(methyl)amino]acetic acid | HMDB00064 |
| 2 | Betaine | 2-(trimethylazaniumyl)acetate | HMDB00043 |
| 3 | Palmitate | | HMDB00220 |
| 4 | 2-hydroxybutyrate | | HMDB00008 |
| 5 | Oleic acid | (Z)-octadec-9-enoic acid | HMDB00207 |
| 6 | Tryptophan | | HMDB00929 |
| 7 | Palmitoleic acid | (Z)-hexadec-9-enoic acid | HMDB03229 |
| 8 | Threonine | | HMDB00167 |
| 9 | Linoleic acid | (9Z,12Z)-octadeca-9,12-dienoic acid | HMDB00673 |
| | | or | |
| | | cis-9,cis-12-octadecadienoic acid | |
| 10 | Decanoyl carnitine | 3-decanoyloxy-4- | |
| | | (trimethylazaniumyl)butanoate | |
| 11 | Arginine | | HMDB00517 |
| 12 | Octanoyl carnitine | 3-octanoyloxy-4- | |
| | | (trimethylazaniumyl)butanoate | |
| 13 | linolenic acid | (9Z,12Z,15Z)-octadeca-9,12,15-trienoic | HMDB01388 |
| | | acid = | |
| | | α-Linolenic acid | |
| 14 | margaric acid | heptadecanoic acid = margarate, margaric | HMDB02259 |
| | | acid | |
| 15 | Serine | 2-amino-3-hydroxy-propanoic acid | HMDB00187 |
| 16 | stearic acid (stearate) | Octadecanoic acid | HMDB00827 |
| 17 | glutamic acid | 2-aminopentanedioic acid | HMDB00148 |
| 18 | Glycine | 2-aminoacetic acid | HMDB00123 |
| 19 | 3-methyl-2-oxo- | 3-methyl-2-oxo-butanoic acid | HMDB04260 |
| | butyric acid | | |
| 20 | linoleoyl | 1-linoleoyl-2-hydroxy-sn-glycero-3- | |
| | lysophosphatidyl | phosphocholine | |
| | choline | 2-linoleoyl-1-hydroxy-sn-glycero-3- | |
| | (Linoleoyl-LPC) | phosphocholine | |
| 21 | oleoyl | 1-oleoyl-2-hydroxy-sn-glycero-3- | |
| | lysophosphatidyl | phosphocholine | |
| | choline | 2-oleoyl-1-hydroxy-sn-glycero-3- | |
| | (Oleoyl-LPC) | phosphocholine | |
| 22 | palmitoyl | 1-palmitoyl-2-hydroxy-sn-glycero-3- | |
| | lysophosphatidyl | phosphocholine | |
| | choline | | |
| | (palmitoyl-LPC) | | |
| 23 | 3-hydroxy-butyrate | 3-hydroxybutanoic acid | HMDB00357 |
| 24 | Docosatetraenoic | (7Z,10Z,13Z,16Z)-docosa-7,10,13,16- | HMDB02226 |
| | acid = | tetraenoic acid | |
| | Adrenic acid | | |
| 25 | 1,5-anhydroglucitol | (2R,3S,4R,5S)-2-(hydroxymethyl)oxane- | HMDB02712 |
| | | 3,4,5-triol | |
| 26 | Stearoyl-LPC | | |
| 27 | Glutamyl valine | | |
| 28 | Gamma-glutamyl- | | |
| | leucine | | |
| 29 | alpha-ketobutyrate | | HMDB00005 |
| 30 | Cysteine | | HMDB00574 |
| 31 | Urate | | HMDB00289 |
| 32 | Isovalerylcarnitine | | HMDB00688 |
| 33 | Myo-inositol | | |
| 34 | 1-palmitoyl- | | |
| | glycerophospho | | |
| | ethanolamine | | |
| 35 | Catechol sulfate | | |
| | Previously unnamed, | | |
| | Metabolite-2272 has | | |
| | been identified as | | |
| | catechol sulfate | | |
| 36 | 3-phenylpropionate | | HMDB00764 |

¹See http://www.hmdb.ca

### 2B: Evaluation of Biomarkers and Development of Models for Insulin Resistance

To evaluate the identified biomarkers, plasma samples were collected from 401 fasting subjects, and the IR Markers and Models described in Tables 4, and 6, respectively, were used to predict the glucose disposal rate of individuals and to predict whether the subject was insulin sensitive or insulin resistant. The predicted glucose disposal rate (Rd) was then used to classify the individuals according to their glucose tolerance as having normal glucose tolerance (NGT), impaired glucose tolerance (IGT) or type 2 diabetes (T2D). The cohort is described in Table 5.

TABLE 5
Cohort Description
Table 5. Cohort description

| Total | | Sex | | | | | | | |
|---|---|---|---|---|---|---|---|---|---|
| Subjects | | Male | Female | Age | | BMI | | Rd | |
| in Study | Group | (N) | (N) | Mean | SD | Mean | SD | Mean | SD |
| 401 | IFG | 56 | 30 | 47.1 | 7.8 | 28.0 | 4.0 | 5.95 | 3.07 |
| | IGT | 23 | 34 | 45.6 | 7.7 | 27.3 | 4.4 | 4.43 | 1.79 |
| | NGT-IR | 20 | 31 | 45.0 | 7.7 | 26.0 | 3.5 | 4.69 | 0.98 |
| | NGT-IS | 97 | 110 | 43.4 | 8.4 | 23.8 | 3.4 | 9.62 | 2.30 |

Abbreviations:
IFG: Impaired Fasting Glucose;
IGT: Impaired Glucose Tolerance;
NGT-IR: Normal Glucose Tolerance-Insulin Resistant;
NGT-IS: Normal Glucose Tolerance-Insulin Sensitive;
BMI: Body Mass Index;
Rd: Glucose Disposal Rate;
SD: Standard Deviation.

Using biomarker 1-25 listed in Table 4, models were generated using two different but similar strategies as described below. The first approach used a variable selection strategy with 3 core variables held constant and other variables added one by one. In the second approach all possible models were generated using biomarkers 1-25 in Table 4. In both approaches each model was tested to assess the impact of variable selection on diagnostic parameters.

The first strategy used a variable/model selection strategy using core variables in Multiple Linear Regression (MLR) analysis. The dataset consisted of 401 samples, and the outcome variable used was the square root of the glucose disposal rate (SQRTRd). This strategy is based on a core of three variables and adds-in variables according to cross-validated performance measures (R-square, Sensitivity, Specificity).

Using a core of variables that included various combinations of body mass index (BMI), 2-hydroxybutyrate, linoleoyl-LPC, decanoyl-carnitine and creatine, one or more of the following compounds can be added to the model with comparable R-square, sensitivity and specificity:
Linoleic acid;
Docosatetraenoic acid;
Glycine;
Margaric acid;
Linolenic acid;
Palmitate;
Tryptophan;
Oleic acid;
3-Methyl-2-oxo-butyric acid;
Stearate.

The second strategy used a variable/model selection strategy using all possible variables in Multiple Linear Regression (MLR) analysis. This strategy also used samples from 401 subjects for the dataset and the square root of the glucose disposal rate (SQRTRd) as the outcome variable. In addition, the analysis employed predictor variables of body mass index (BMI) plus 25 LC targeted assays developed to measure the 25 biomarker compounds to construct the best 10,000 possible MLR models having 5 and 6 variables. After the initial 10,000 models were identified, models were selected with all individual p-values less than or equal to 0.05 (<0.05).

Modeling with 5,000 possible multiple linear regression models produced a total of 1,502 models with 5 variables and 862 models with 6 variables with the following 6 models dominant:
1. BMI, 2-hydroxybutyrate, linoleoyl-LPC, decanoyl-carnitine, palmitate, palmitoleic acid (occurrence or n=332 out of 5,000 models)
2. BMI, 2-hydroxybutyrate, linoleoyl-LPC, decanoyl-carnitine, threonine, linoleic acid (n=142 out of 5,000 models)
3. BMI, 2-hydroxybutyrate, linoleoyl-LPC, decanoyl-carnitine, threonine, glycine (n=80 out of 5,000 models)
4. BMI, 2-hydroxybutyrate, linoleoyl-LPC, decanoyl-carnitine, threonine, stearate (n=54 out of 5,000 models)
5. BMI, 2-hydroxybutyrate, linoleoyl-LPC, decanoyl-carnitine, 3.methyl.2.oxo.butyric acid, linoleic acid (n=79 out of 5,000 models)
6. BMI, 2-hydroxybutyrate, linoleoyl-LPC, decanoyl-carnitine, 3.methyl.2.oxo.butyric acid, docosatetraenoic acid (n=51 out of 5,000 models)

Two of the best 6-variable models consisting of BMI, 2-hydroxybutyrate, linoleoyl-LPC, decanoyl-carnitine, creatine, and palmitate or stearate (Table 6) showed similar test performance characteristics in the whole study population (n=401) or the At Risk Population (n=275). The “At Risk” population is a subset of the study population that are considered to be at risk of having insulin resistance based on ADA guidelines for the identification of people having insulin resistance. Logistic regression modeling preferred the 6-variable model that included stearate over the model that included palmitate.

TABLE 6
Rd Regression Model (Cut-off 6) Whole (n = 401) vs. At Risk (n = 275)

| | Model | | | |
|---|---|---|---|---|
| | BMI | | BMI | |
| | 2-Hydroxybutyrate | | 2-Hydroxybutyrate | |
| | Decanoyl-carnitine | | Decanoyl-carnitine | |
| | Linoleoyl-LPC | | Linoleoyl-LPC | |
| | Creatine | | Creatine | |
| | Palmitate | | Stearate | |
| Population | Whole | At Risk | Whole | At Risk |
| R-square | 0.482 | 0.473 | 0.481 | 0.476 |
| AUC | 0.767 | 0.771 | 0.773 | 0.802 |
| Sensitivity (%) | 63.19 | 69.92 | 63.89 | 71.54 |
| Specificity (%) | 90.27 | 84.21 | 90.66 | 88.82 |
| PPV* (%) | 78.45 | 78.18 | 79.31 | 83.81 |
| NPV* (%) | 81.40 | 77.58 | 81.75 | 79.41 |
| DLR+: Sen/(1 − Spec) | 6.494 | 4.428 | 6.840 | 6.399 |
| DLR−: (1 − Sen)/Spec | 0.408 | 0.357 | 0.398 | 0.320 |
| Pre-test IR Odds* | 0.560 | 0.809 | 0.560 | 0.809 |
| Post-test IR Odds+* | 3.639 | 3.583 | 3.833 | 5.178 |
| Odds ratio: | 15.92 | 12.39 | 17.17 | 19.97 |
| DLR+/DLR− | | | | |

The positive predictive value (PPV) and negative predictive value (NPV) values in Table 6 were obtained from the dataset but they may differ since they depend on the prevalence of the disease. The same was true for the Pre-test Odds values. A DLR+ of 6.5 means that a positive test was 6.5 times more likely in an IR subject than in an IS subject. Also, it shows how much the post-test odds increased from the pre-test odds. Pre-test odds are the odds of a subject being IR before the diagnostic test is taken. Post-test odds are the odds of a subject being IR after the diagnostic test. DLR− was calculated as (1-Sen)/Spec. A value of 0.4 means that a negative test was 2.5 times less likely in an IR subject than in an IS subject. Post-test IR Odds were calculated similarly. Finally the Odds ratio can be calculated as DLR+/DLR−(6.5/0.4=16.25) and it means that the IR odds are 16 fold greater for a positive test than for a negative test.

To predict the glucose disposal (Rd) based on biomarkers 1-25 in Table 4, a regression model was used with the square root of Rd as the dependent variable and the values of six independent variables, including BMI. The regression model was built using a forward selection model on a different data set with 401 observations.

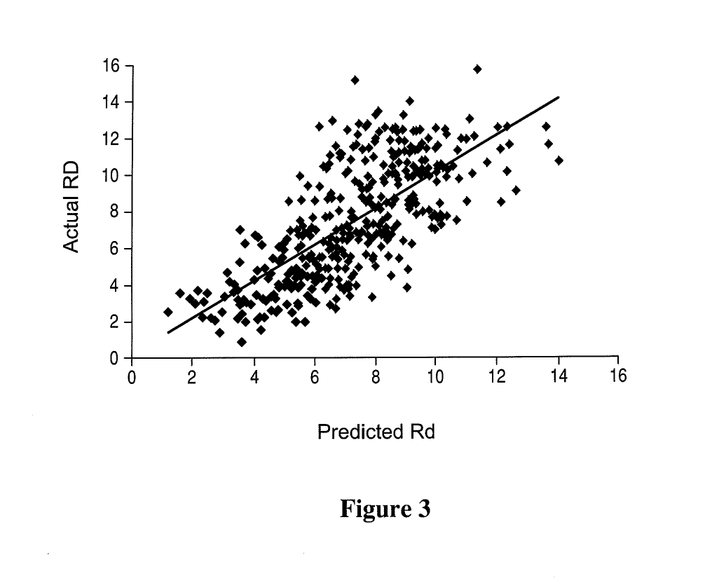

Predictions were obtained by substituting the measured values of the six variables from the data set into the regression equation. Since the predicted value is the square root of Rd, the predicted values were subsequently squared. FIG. 3 provides an example of the correlation of actual glucose disposal (Rd) and predicted Rd based on measuring biomarkers in plasma collected from a group of 401 insulin resistant subjects.

### 2C: Model Variations

Other models with or without BMI or C-peptide were developed that suggested that C-peptide could replace BMI in the models (see Model 1 compared to Model 4). The four models were as follows:
(#1) BMI, 2-Hydroxybutyrate, Linoleoyl-LPC, Decanoyl-carnitine, Creatine, Palmitate (Original Model)
(#2)2-Hydroxybutyrate, Linoleoyl-LPC, Decanoyl-carnitine, Creatine, Palmitate (Model #1 Without BMI)
(#3) BMI, C-peptide, 2-Hydroxybutyrate, Linoleoyl GPC, Decanoyl-carnitine, Palmitate (#1 plus Fasting C-peptide)
(#4) C-peptide, 2-Hydroxybutyrate, Linoleoyl GPC, Decanoyl-carnitine, Palmitate (#1 Without BMI but with C-peptide)

The results of each model are shown in the tables below. In each table the Rd cut-off for insulin resistance was varied. The most widely used and accepted cut-off is an Rd of 6.0 (Cut 6 in Table 8), in which subjects with an Rd>6 are considered insulin sensitive and subjects with an Rd<6 are considered insulin resistant. To determine the effects of increasing or decreasing the Rd cut-off on test performance the analysis was carried out at an Rd of 5 (Cut 5, Table 7) and an Rd of 7 (Cut 7, Table 9). While some models performed better than others, each model provided the ability to determine insulin resistance in subjects at each of the selected Rd cut-off values and with clinically acceptable values of the diagnostic parameters (AUC, Sensivity, Specificity, Negative Predictive Value and Positive Predictive Value).

TABLE 7
Diagnostic Parameters of Models with Rd Cut-off Value of 5.

| | Cut 5 | | | | | |
|---|---|---|---|---|---|---|
| | Rsq | AUC | Sen | Spec | PPV | NPV |
| Model # 1 | 0.482 | 0.712 | 46.85% | 95.52% | 80.00% | 82.44% |
| Model # 2 | 0.347 | 0.650 | 34.23% | 95.86% | 76.00% | 79.20% |
| Model # 3 | 0.513 | 0.715 | 47.75% | 95.16% | 79.10% | 82.58% |
| Model # 4 | 0.465 | 0.714 | 46.85% | 95.85% | 81.25% | 82.44% |

TABLE 8
Diagnostic Parameters of Models with Rd Cut-off Value of 6.

| | Cut 6 | | | | | |
|---|---|---|---|---|---|---|
| | Rsq | AUC | Sen | Spec | PPV | NPV |
| Model # 1 | 0.482 | 0.767 | 63.19% | 90.27% | 78.45% | 81.40% |
| Model # 2 | 0.347 | 0.737 | 58.33% | 89.11% | 75.00% | 79.24% |
| Model # 3 | 0.513 | 0.783 | 66.67% | 89.84% | 78.69% | 82.73% |
| Model # 4 | 0.465 | 0.776 | 64.58% | 90.63% | 79.49% | 81.98% |

TABLE 9
Diagnostic Parameters of Models with Rd Cut-off Value of 7.

| | Cut 7 | | | | | |
|---|---|---|---|---|---|---|
| | Rsq | AUC | Sen | Spec | PPV | NPV |
| Model # 1 | 0.482 | 0.795 | 75.63% | 83.33% | 81.42% | 77.98% |
| Model # 2 | 0.347 | 0.760 | 73.10% | 78.92% | 77.01% | 75.23% |
| Model # 3 | 0.513 | 0.800 | 76.65% | 83.25% | 81.62% | 78.60% |
| Model # 4 | 0.465 | 0.792 | 72.59% | 85.71% | 83.14% | 76.32% |

### Example 3

### The Predicted Rd is Useful to Generate an IR Score

Glucose disposal rates (Rd) predicted using the biomarkers and models identified above are useful to determine the probability of insulin resistance in a subject. An “IR Score” can be generated that provides the probability that an individual is insulin resistant. The higher the Rd, the lower the probability of insulin resistance and the lower the IR score. Conversely, the lower the Rd, the higher the probability that the individual is insulin resistant and the higher the IR score. Several methods can be used to determine the probability of insulin resistance.

### 3A: Probability Score Algorithm

A standard probability curve for predicting insulin resistance in a subject was then generated using a probability score algorithm. To obtain the “probability score,” the predicted values and individual prediction errors (not the predicted error of the mean) were obtained from the regression model used to generate the predicted glucose disposal rate. An individual's values were then treated as a normal random variable with a mean equal to the predicted value and standard deviation equal to the prediction error. Then the probability was obtained by computing the probability that a normal random variable with the mean and standard deviation above was less than the square root of six.

For regression analysis two error measurements are typically associated with a predicted value. One measure is the standard error of the mean. This value was used to set up confidence intervals for the true mean value. A 95% confidence interval means that 95% of the time the procedure will produce an interval that contains the true mean. A second measure of error for the prediction is the prediction error. This relates to an individual rather than a mean. A 95% prediction interval means that 95% of the time the procedure will produce an interval that contains a future observation.

Then the formulas for these errors were as follows:

(1) Standard error is the square root of x′₀(X′X)⁻¹x₀s²

(2) Prediction error is the square root of s^{2[}1+x′₀(X′X)⁻¹x₀],

where X is the matrix of all of the predictors, s² is the MSE (mean squared error), and x′ is the vector of values for the predictor values (with a 1 for the intercept) for one individual. (The formulas are taken from Rawlings, O., Pantula, S., Dickey, D., Applied Regression Analysis, page 146, 1998, Springer-Verlag New York Inc.)

For the probability score calculation developed above, a normal distribution was assumed for an individual with the predicted value as the mean and the prediction error as the standard deviation. Then the probability that this random variable is less than six was calculated. Thus, the calculation was Prob((6-predicted value)/prediction error>0) using the standard normal distribution. Since the response in the final model was the square root of R_{d}, the above changes to the square root of six.

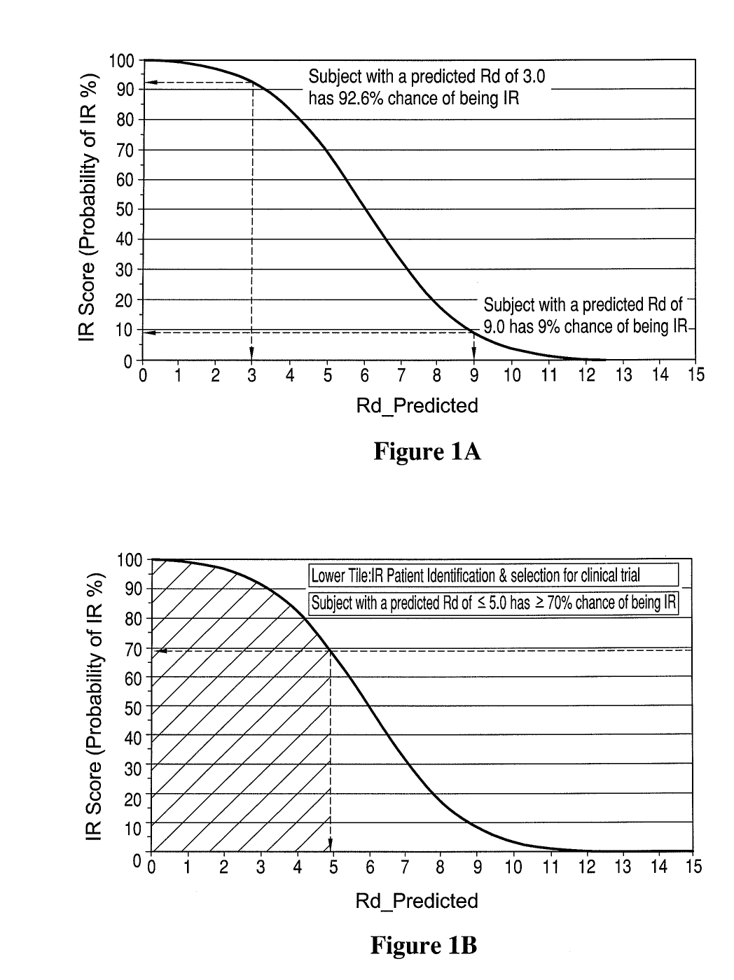

A standard probability curve was then generated which can be used to predict a subject's probability of having IR (or IR Score) based on the predicted glucose disposal rate using the models disclosed herein. A standard curve is provided in FIG. 1A, which can be used to determine an individual's IR Score. For example, as shown in FIG. 1A, a subject having a predicted Rd of 9, can be plotted against the standard curve, and then identified as having an IR Score of 10. The IR Score of 10 indicates that the subject has a 10% probability of having insulin resistance. Alternatively, a subject having a predicted Rd value of 3, can be identified as having an IR Score of 90 by plotting the value against the standard curve. The subject's score of 90 indicates that the subject has a 90% probability of having insulin resistance.

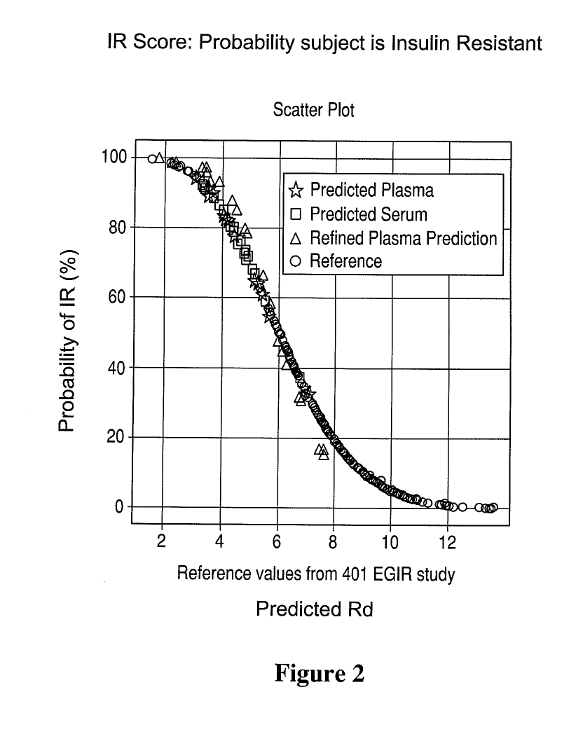

Serum and plasma samples collected at baseline from 23 male and female type II diabetics in a phase I clinical trial were analyzed using insulin resistance biomarkers 1-25 in Table 4. The measured levels of the panel of biomarkers obtained from this targeted analysis were used to calculate a predicted Rd and an associated IR score (probability of IR) for each subject. These calculations used a model as described above and were plotted on the reference curve illustrated in FIG. 2. As illustrated in FIG. 2, most of the predicted values from this model fell in the expected range (Rd=0 to 6) for insulin resistant subjects and indicated the probability the subjects were insulin resistant. The results were within the predicted sensitivity and specificity of the assay.

For certain subjects the correlation of the predicted Rd value with the measured Rd was not as high as previously obtained with a non-diabetic cohort. Another model was developed by using the measured Rd values in forward selection regression models. The correlation between the measured and predicted Rd was improved and the median absolute error was reduced to 0.81 using a refined model with 3 variables (oleoyl-LPC, creatine, and decanoyl-carnitine). Thus, biomarkers 1-25 in Table 4 are very useful for predicting insulin resistance (e.g. via modeling of one or more of the biomarkers) in diabetic subjects as well as in pre-diabetic subjects.

### 3B: Logistic Regression to Generate an IR Score

A logistic regression analysis was performed as another method to compute a probability score. Logistic regression models the probability in terms of model with the predictors, e.g., let Y=0 be the event that Rd≧6 and Y=1 be the event that Rd<6. The logistic regression model is Prob(Yj=1)=exp(b₀+b₁x₁+b₂x₂+ . . . +bₚxₚ)/(1+exp(b₀+b₁x₁+b₂x₂+ . . . +bₚxₚ), where bᵢ is the coefficient and xᵢ is the value of the i^{th} predictor variable for the j^{th} subject. An example using this method with one of the models generated from the IR Biomarkers Panel is described below.

In this example, the predictors were: Let Y=0 be the event that Rd≧6 and Y=1 be the event that Rd<6. The logistic regression model is Prob(Yj=1)=exp(b₀+b₁x₁+b₂x₂+ . . . +bₚxₚ)/(1+exp(b₀+b₁x₁+b₂x₂+ . . . +bₚxₚ), where bᵢ is the i^{th} coefficient and xᵢ is the value of the i^{th} predictor variable for the j^{th} subject. For the 401 subject data set, the model containing oleoyl-GPC was selected instead of linoleoyl-GPC. Palmitate was not significant using the Likelihood Ratio Test (Table 11), so it was dropped from the model. The model was fitted with JMP (SAS Institute, Inc., Cary, N.C.). The coefficients used are provided in Table 10, below.

TABLE 10
Coefficients for the Logistic Regression Model

| | | Std | | |
|---|---|---|---|---|
| Term | Coefficient | Error | Chi-Sq | p-value |
| Intercept | −8.39967 | 1.411962 | 35.38985 | <0.0001 |
| BMI | 0.241821 | 0.040489 | 35.67024 | <0.0001 |
| 2-hydroxybutyrate | 0.579104 | 0.097499 | 35.2786 | <0.0001 |
| oleoyl-GPC | −0.13138 | 0.047544 | 7.63558 | 0.0057 |
| decanoyl_carnitine | −10.4667 | 3.995164 | 6.863571 | 0.0088 |
| Creatine | 0.178803 | 0.067436 | 7.030164 | 0.0080 |

TABLE 11
Effect Likelihood Ratio Tests

| | | | L-R | |
|---|---|---|---|---|
| Source | Nparm | DF | ChiSq | p-value |
| BMI | 1 | 1 | 44.65507 | <0.0001 |
| 2-hydroxybutyrate | 1 | 1 | 43.39072 | <0.0001 |
| oleoyl-GPC | 1 | 1 | 8.103003 | 0.0044 |
| decanoyl_carnitine | 1 | 1 | 8.530153 | 0.0035 |
| Creatine | 1 | 1 | 7.243963 | 0.0071 |

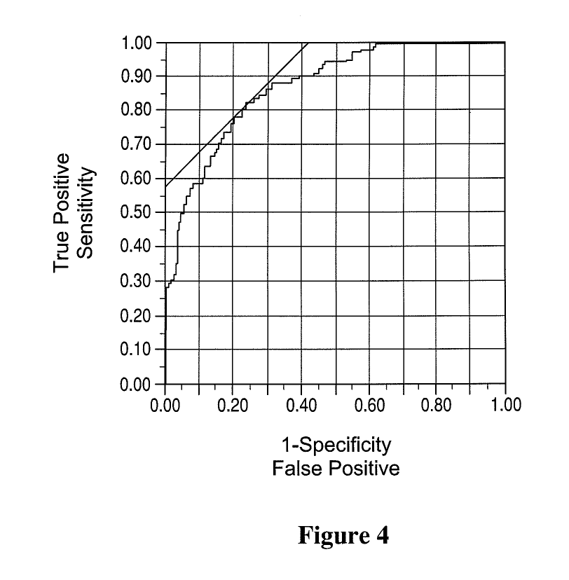

The Receiver Operating Characteristic (ROC) Curve is provided in FIG. 4, where the area under the curve (AUC) was 0.87155.

The following model was used on the cohort described in Table 5 to determine if a given subject was insulin sensitive (HIGH) or insulin resistant (LOW):

Prob(Rd<6)=exp(−8.3997+0.2418*BMI+0.5791*2-hydroxybutyrate−0.1314*oleoyl-GPC−10.4667*decanoylcarnitine+0.1788*creatine)/(1+exp(−8.3997+0.2418*BMI+0.5791*2-hydroxybutyrate−0.1314*oleoyl-GPC−10.4667*decanoylcarnitine+0.1788*creatine).

The results using this model are presented in the table (Table 12) below. Subjects described as LOW are “positive” for insulin resistance (i.e. the subject is insulin resistant) and subjects described as HIGH are “negative” for insulin resistance (i.e. the subject is insulin sensitive).

TABLE 12
Confusion Matrix:

| | | TEST | |
|---|---|---|---|
| | | LOW | HIGH |
| ACTUAL | LOW | 92 | 51 |
| | HIGH | 33 | 225 |

The model has a sensitivity of 64%, a specificity of 87%, an PPV of 74%, and an NPV of 82%.

### Example 4

### Patient Stratification for Treatment and Clinical Trials Based Upon Predicted Rd and Associated IR Score

Identification of Insulin Resistant Subjects based on the IR score can be used to identify subjects for Insulin-sensitizer Treatment, subject stratification for identifying IR-T2D and IR-pre-diabetics with fasted blood sample, and measuring IR.

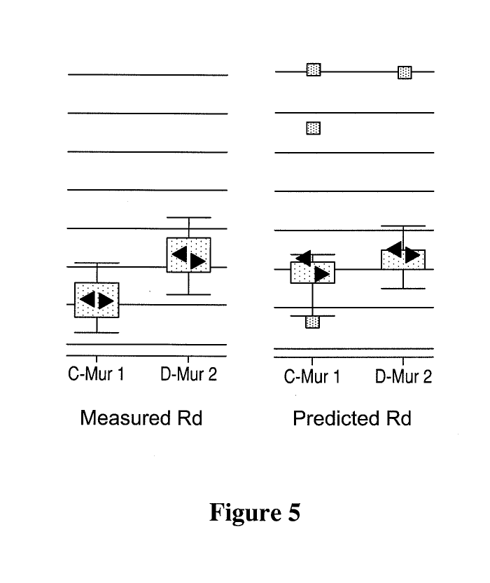

Type-2 diabetes mellitus (T2DM) prevention trials have demonstrated the significance of IR due to consistent trends of insulin sensitizers in successful prevention. Biomarkers 1-25 listed in Table 4 were measured in plasma samples collected from 16 subjects that were taking the insulin sensitizer muraglitozar. The samples were collected pre-(C-Mur_{—}1) and post-treatment (D-Mur_{—}2) with muraglitozar. As shown in FIG. 5, the changes in the predicted Rd (Right panel) determined based upon biomarkers 1-25 in Table 4 increased with treatment to the insulin sensitizer, which is in agreement with the actual Rd measured by the HI clamp (Left panel).

### 4A: Use of the Predicted Rd and IR Score to Identify High-Risk IR Subjects for Insulin Sensitizer Class Drugs

As mentioned above, it is known that the more insulin resistant a subject is, the greater the response to an insulin sensitizer compound the subject will have. Thus, the generation of an IR Score can be used to identify high-risk IR subject for treatment with insulin sensitizer compositions.

For example, using the biomarkers and models provided herein, subjects can be identified that may be good candidates for insulin sensitizer therapeutics. As shown in FIG. 1B, a subject having a predicted glucose disposal rate of less than or equal to 5 would have a greater or equal to 70% chance of being insulin resistant. Such individuals could then be selected for insulin sensitizer treatment or selected for acceptance into clinical trials.

4B: Classification of Subjects Based on IR Biomarkers and Comparison with OGTT and FPG Test Results

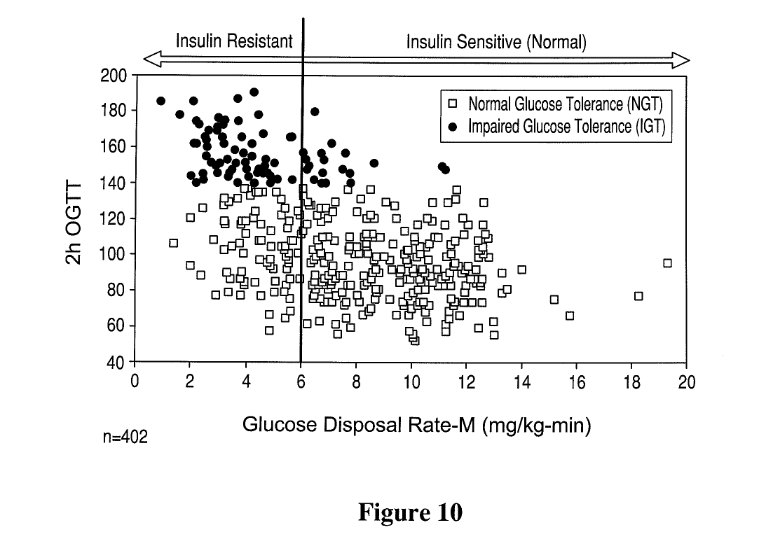

The 2h OGTT and glucose disposal (M) values for each of 401 subjects selected from the cohort described in Table 5 were plotted in FIG. 10. The data shows that some insulin resistant (IR) individuals may have normal glucose tolerance (NGT) as measured by the 2h OGTT while some of the impaired glucose tolerance (IGT) subjects may have normal insulin sensitivity.

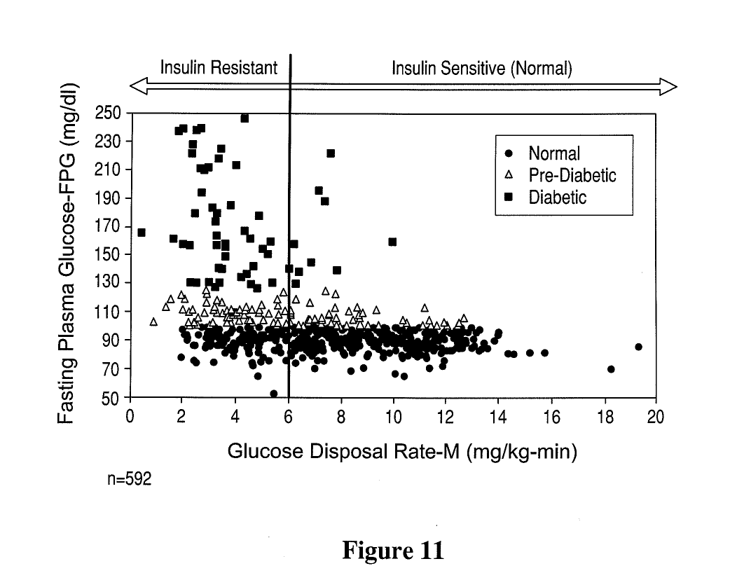

The fasting plasma glucose and M values for each of 592 subjects were plotted in FIG. 11 The data shows that fasting plasma glucose may be within normal levels (≦100 mg/dl) in an IR subject. Thus, some individuals may appear to have normal glucose levels but are actually pre-diabetic when the IR status is taken into account. Furthermore, some of the subjects classified as diabetic and pre-diabetic based upon fasting plasma glucose measurements may be insulin sensitive (i.e., normal).

### Example 5

### Comparison of Biomarkers and Algorithms to Current Clinical Tests for Glucose Tolerance and Type-2 Diabetes

The performance of IR Biomarkers Model was compared with the results of the OGTT and FPG test in the cohort of 401 subjects described in Table 5. The IR Biomarkers Model had better Sensitivity, Specificity, Positive Predictive Value and Negative Predictive Value than either of the other currently used clinical tests. The results of the comparison of IR biomarkers with clinical assays currently used to measure insulin resistance and type 2 diabetes are summarized in Table 13.

TABLE 13
Comparison of IR Biomarkers in instant application with Clinical
Assays currently used to measure insulin resistance and type 2 diabetes

| TEST | Sensitivity (%) | Specificity (%) | PPV (%) | NPV (%) |
|---|---|---|---|---|
| IR Biomarkers | 62.2 | 93.8 | 83.2 | 83.3 |
| Model | | | | |
| OGTT | 46.2 | 92.5 | 75.3 | 77.6 |
| FPG | 33.6 | 85.5 | 56.1 | 50.0 |

Plasma samples from a subset of subjects described in Table 5 that had data available for insulin, glucose disposal (Rd), adiponectin and results from the OGTT and HOMA-IR tests were evaluated for the correlation with Rd, the glucose disposal rate measurement obtained from the HI clamp. A total of 369 plasma samples from 369 subjects were analyzed. Subjects that had missing values were not included; 14 subjects were missing Fasting Insulin values and 2 additional subjects were missing values for adiponectin. These results and the result obtained on the same 369 subjects with the IR Model: SQRTRD˜BMI+2 Hydroxybutyrate+Linoleate (x)+Linolyl_GPC+decanoylcarnitine are shown in Table 14. The IR Model was significantly correlated (p-value=2.01E-54) with Rd and showed a better R value than did any of the other markers or models. The IR Model also had better diagnostic performance based upon the AUC, Sensitivity, Specificity, Negative Predictive Value and Positive Predictive Value than any of the other tests. In addition, the biomarkers and models provided herein demonstrate a similar correlation with glucose disposal than the HI clamp.

TABLE 14
Comparison of IR model with other commonly used tests, algorithms
and biomarkers to determine insulin sensitivity in a subject.

| Dx Test | N | R | P-value | AUC | Sens | Spec | NPV | PPV |
|---|---|---|---|---|---|---|---|---|
| IR Model | 369 | 0.71 | 2.01E−54 | 74.8 | 59.5 | 90.1 | 75.8 | 81.1 |
| OGTT | 369 | NA | NA | 68.0 | 43.7 | 92.2 | 74.3 | 75.9 |
| FPG | 369 | −0.16 | 0.002072 | 58.7 | 31.8 | 85.6 | 53.3 | 70.8 |
| HOMA-IR | 369 | −0.56 | 1.44E−31 | 70.0 | 50.8 | 89.3 | 71.1 | 77.8 |
| Adiponectin | 369 | 0.31 | 7.44E−10 | 57.6 | 35.0 | 80.3 | 47.8 | 70.4 |

### Example 6

### Monitoring Insulin Resistance Following Bariatric Surgery

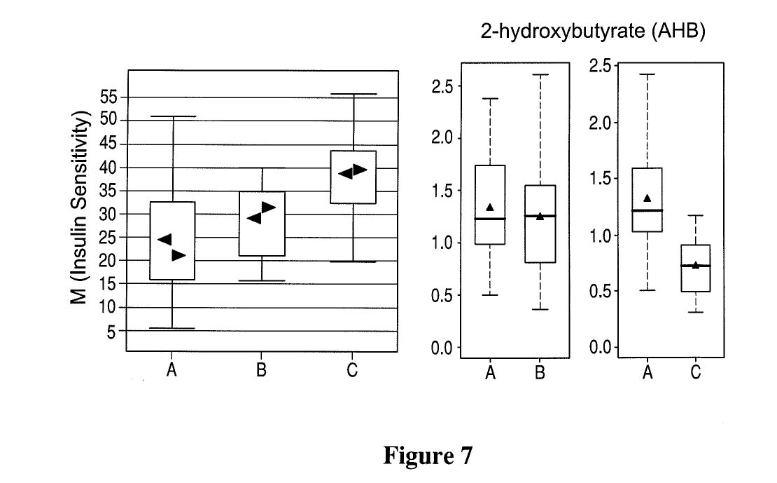

Plasma samples were collected from 105 subjects at three time-points for metabolic profiling. The plasma samples were collected at baseline (“A”, pre-surgery; n=43), post-surgery, pre-weight loss (“B”, approximately 3.4 months after surgery; n=27), and post-surgery, post-weight loss (“C”, approximately 16.4 months after surgery; n=35). As measured by the hyperinsulinemic euglycemic clamp method, insulin sensitivity improves after surgery and prior to weight loss for many subjects. As shown in FIG. 7, 2-Hydroxybutyrate (2HB) levels decreased as insulin sensitivity increases in these subjects. For many subjects insulin sensitivity improves prior to weight loss (FIG. 7, left panel) while 2HB is reduced post-bariatric surgery (FIG. 7, right panel) and the reduction becomes more pronounced with weight loss. In addition, ratios of metabolites, such as lactate, do not have such pronounced improvements.

In addition to 2HB, the levels of other IR biomarkers are also changed following bariatric surgery. The table below (Table 15) shows that the levels of biomarkers 1-25 in Table 4 show the expected change in bariatric surgery subjects post-surgery and following weight loss, when patients have become less insulin resistant.

TABLE 15
Changes in IR Biomarkers following bariatric surgery.

| BIOMARKER | p-value | avg_A | avg_C |
|---|---|---|---|
| Glutamic acid | 3.04E−10 | 42.1438 | 25.03594 |
| 2-hydroxybutyrate | 6.9E−09 | 7.054138 | 4.036692 |
| Linolenic acid | 1.92E−07 | 2.165038 | 1.44484 |
| Tryptophan | 4.96E−06 | 11.65414 | 9.467885 |
| Stearic acid (Stearate) | 1.31E−05 | 14.07902 | 11.43068 |
| Glycine | 2.93E−05 | 14.07231 | 16.7935 |
| Palmitoyl-LPC | 4.87E−05 | 32.87684 | 26.17387 |
| Creatine | 0.000309 | 6.707253 | 4.902922 |
| Margaric acid | 0.003052 | 0.499124 | 0.418034 |
| Palmitate | 0.003538 | 44.98923 | 37.27746 |
| Octanoyl carnitine | 0.008151 | 0.031148 | 0.025135 |
| Linoleic acid | 0.010973 | 20.56562 | 17.47978 |
| Decanoyl carnitine | 0.017438 | 0.05137 | 0.042486 |
| Serine | 0.018057 | 14.14932 | 15.15901 |
| Palmitoleic acid | 0.021881 | 4.60217 | 3.926733 |
| 1-5-anhydroglucitol | 0.027855 | 25.26796 | 19.33572 |
| 3-hydroxy-butyrate | 0.039985 | 9.490739 | 14.10837 |
| 3-methyl-2-oxo-butyric | 0.042559 | 0.763369 | 1.011802 |
| acid | | | |
| Docosatetraenoic acid | 0.059759 | 0.355993 | 0.416227 |
| Betaine | 0.0734 | 4.191903 | 3.823797 |
| Threonine | 0.157488 | 15.80487 | 14.63062 |
| Linoleoyl-LPC | 0.164853 | 10.91704 | 10.3207 |
| Oleic acid | 0.333929 | 117.3331 | 111.9031 |
| Arginine | 0.43392 | 17.45961 | 17.77525 |
| Oleoyl-LPC | 0.755347 | 7.451764 | 7.515243 |

A, baseline levels prior to surgery.
C, levels post-surgery, post-weight loss when subjects are less insulin resistant.

The glucose disposal rate (Rd) of subjects at baseline (A) and after weight loss (C) was predicted using the IR Biomarkers (Tables 4A and 4B) in an IR Model. The predicted Rd in the subjects at time C was higher (4.14) than that at time A (0.783), and the predictions were statistically significant (p-value=4.45E-09) indicating that the sensitivity of the subjects to insulin was increased, that is, the subjects became less insulin resistant. This is consistent with the Rd measurement of insulin sensitivity obtained with the hyperinsulinemic euglycemic clamp data shown above. Thus, the IR Biomarkers in Tables 4A and 4B can be used to determine changes in insulin resistance in subjects following a lifestyle intervention, in this case bariatric surgery.

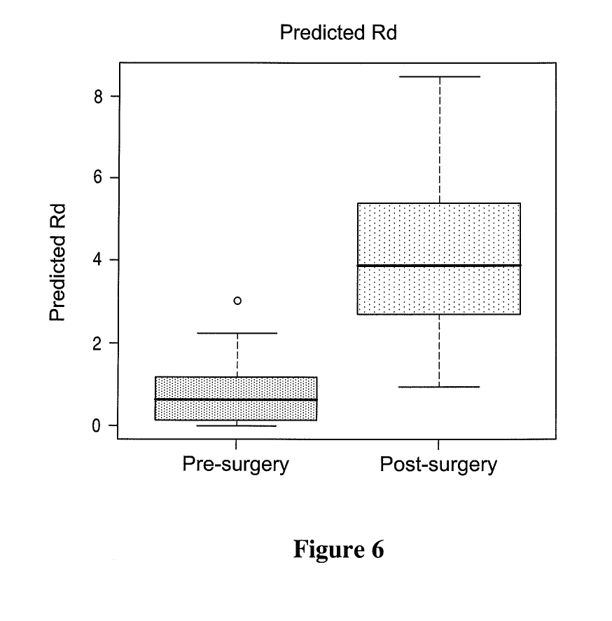

As shown in FIG. 6, the predicted Rd using a model of biomarkers listed in Tables 4A and 4B is consistent with measured Rd values using the HI clamp. In addition, FIG. 6 shows that the predicted Rd is low at the baseline (pre-surgery) when subjects are insulin resistant and that the levels increase post-surgery, post-weight loss (post-surgery) when subjects are less insulin resistant.

### Example 7

### Identification of IR Target Compositions Effecting Biochemical Pathways

The biomarkers identified in the present application can be used to identify additional biomarkers correlated with insulin resistance, or may used to identify therapeutic compositions capable of modifying the levels of one or more of the disclosed biomarkers by affecting the biochemical pathway(s) in which the biomarkers are involved. The additional biomarkers may be related to the disclosed biomarkers as upstream or downstream in a given biochemical pathway, or a related pathway.

### 7A: 2-Hydroxybutyrate

The levels of 2-hydroxybutyrate (2HB) change in subjects after bariatric surgery. FIG. 7 shows that the levels of 2HB reduce in subjects from baseline (A), to post-surgery, post-weight loss (C). The biochemical 2-hydroxybutyrate (2HB) and related biochemicals and biochemical pathways represent additional biomarkers for insulin resistance, as well as therapeutic agents and drug targets useful for treatment of IR and Type 2 Diabetes. 2-hydroxybutyrate is not considered a ketone body and it does not derive from acetyl-CoA. The three known ketone bodies are acetone, acetoacetic acid, and 3-hydroxybutryic acid. 2HB is found with increased breakdown of amino acids (Met, Thr, a-amino butyrate). 2HB is a marker of hepatic glutathione synthesis during conditions of chronic oxidative stress.

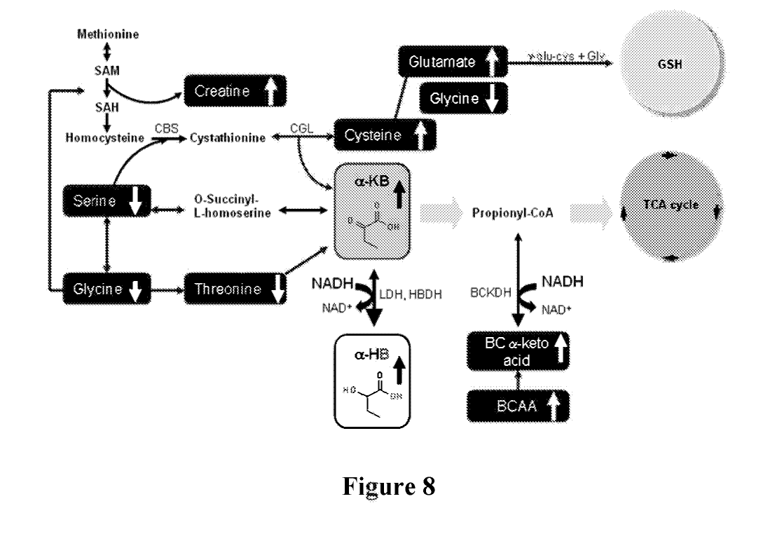

Biochemically, 2HB conventionally known to be produced directly from 2-ketobutyrate, also called alpha-ketobutyrate. (See FIG. 8). Homocysteine is diverted into the trans-sulfuration pathway to form cysteine for sustaining glutathione levels, and 2-ketobutyrate. 2 KB is also formed from the catabolism of threonine and methionine (FIG. 8). The substrates and enzymes in the pathways depicted in FIG. 8 and related pathways are candidates for therapeutic intervention and drug targets. For example, inhibition of lactate dehydrogenase (LDH) or activation of hydroxybutyric acid dehydrogenase (HBDH) or branched chain alpha-keto acid dehydrogenase (BCKDH, see below) could prove therapeutic for treatment of insulin resistance.

Similarly, 2HB is also involved in the citric acid cycle (TCA cycle). As shown in FIG. 8, 2HB production is increased when the flux into the TCA cycle, for example, from 2 KB, is reduced. Thus, subtle alterations in energy metabolism (e.g. change in NADH/NAD+ratio) would impact the TCA cycle flux, and would therefore increase production of 2HB. Lactate dehydrogenase (LDH) levels increase during insulin resistance, and LDH isozyme redistribution in muscle also occurs in diabetic studies. In addition, overexpression of LDH activity interferes with normal glucose metabolism and insulin secretion in the islet beta-cell type. Thus, the metabolites, agents, and/or factors related to 2HB in the TCA cycle may also be useful as biomarkers of insulin resistance or could prove therapeutic for the treatment of insulin resistance.

In addition, metabolites and biochemical pathways related to 2HB may be useful in the methods of the present invention. For example, alpha-ketoacids such as 3-methyl-2-oxobutyrate and 3-methyl-2-oxovalerate may be useful. 3-methyl-2-oxobutyrate levels increase in progressive insulin resistant states. Both 3-methyl-2-oxobutyrate (from valine) and 3-methyl-2-oxovalerate (from isoleucine) are significant by t-test.

Furthermore, dehydrogenases are particularly sensitive to the changes in energy metabolism that occur with conditions such as insulin resistance (e.g. to produce inhibition by NADH). Thus, slight elevations in the NADH/NAD+ratio may be expected in the insulin resistant state due to events such as high lipid oxidation.

### Example 8

### Targeted Assays for the Determination of the Level of Biomarkers in Human Plasma by LC-MS-MS

A method for measuring each of the biomarkers listed in Table 16A in EDTA human plasma was developed. Human plasma samples were spiked with internal standards and subjected to protein precipitation as described below. Following centrifugation, the supernatant was removed and injected onto a Waters Acquity/Thermo Quantum Ultra LC-MS-MS system using four different chromatographic systems (column/mobile phase combinations).

The peak areas of the respective parent or product ions were measured against the peak area of the respective internal standard parent or product ions. Quantitation was performed using a weighted linear least squares regression analysis generated from fortified calibration standards prepared immediately prior to each run.

Samples were prepared by adding study samples to individual wells of a 96-well plate. In addition, calibration, blank sample, blank-IS samples, and quality control samples are also included in the 96-well plate. Calibration standards were prepared by adding Combined Calibration Spiking Solutions to water. Calibration standard target concentrations for the various compounds are indicated in Table 16B. Then, acetonitrile/water/ethanol (1:1:2) is added to each of the wells, and a combined internal standard working solution is added to each of the study samples, as well as to the control, calibration standards, and the blank-IS sample. Methanol is added to each sample, shaken vigorously for at least 2 minutes and inverted several times to ensure proper mixture. The samples are then centrifuged at 3000 rpm for 5 minutes at room temperature until a clear upper layer is produced. The clear organic supernatant was transferred to a clean autosampler vial and used for analysis by LC-MS-MS as provided below.

Instrument Conditions for LC-MS-MS:

Compound Set 1 (palmitate (16:0), docosatetraenoic acid, oleate (18:1(n-9))+1359, stearate (18:0), margarate (17:0), linoleate (18:2(n-6)), linolenate (18:2(n-6)), pamitoleic acid, cis-10-heptadecenoic acid):

### Mass Spec Conditions for Compound Set 1

Source Type: HESI source
Monitor: Selected Reaction Monitoring (SRM), negative mode

### Chromatographic Conditions for Compound Set 1 Mobile Phase A1:

### Water/Ammonium Bicarbonate, 500:1

### Mobile Phase B1: ACN/MeOH (1:1)

### Isocratic:

| Time [min] | % A | % B | Flow [mL/min] |
|---|---|---|---|
| 0 | 15 | 85 | 0.5 |

HPLC Column Acquity C 18 BEH, 1.7 micron 2.1 × 100 mm, Waters

### Target Needle Wash Procedure

Use Isopropanol with a target flush volume of 0.500 mL for strong solvent wash and water for the weak solvent wash post-wash.
Compound Set 2 (2-hydroxybutyrate, 3-methyl-2-oxobutyrate, 3-hydroxybutyrate):
Mass Spec Conditions for Compound Set 2 Source Type: HESI source
Monitor: Selected Reaction Monitoring (SRM), negative mode

### Chromatographic Conditions for Compound set 2 (

Mobile Phase A2: Water 0.01% Formic acid

### Mobile Phase B1: ACN/MeOH (1:1)

### Gradient:

| Time [min] | % A | % B | Flow [mL/min] | Profile |
|---|---|---|---|---|
| 0 | 99 | 1 | 0.4 | |
| 1.0 | 60 | 40 | 0.4 | 6 |
| 1.4 | 60 | 40 | 0.4 | 6 |
| 1.5 | 99 | 1 | 0.4 | 6 |

HPLC Column: Acquity C 18 BEH, 1.7 micron 2.1 × 100 mm, Waters

### Target Needle Wash Procedure

Use Isopropanol with a target flush volume of 0.500 mL for strong solvent wash and water for the weak solvent wash post-wash.
Compound Set 3 (linoleoyl-lyso-GPC, oleoyl-lyso-GPC, palmitoyl-lyso-GPC, stearoyl-lyso-GPC, octanoyl carnitine, decanoyl carnitine, creatine, serine, arginine, glycine, betaine, glutamic acid, threonine, tryptophan, gamma-glutamyl-leucine, glutamyl-valine):

### Mass Spec Conditions for Compound Set 3

Source Type: HESI source
Monitor: Selected Reaction Monitoring (SRM), positive mode

### Chromatographic Conditions for Compound Set 3

Mobile Phase A2 Water 0.01% Formic acid
Mobile Phase B2 ACN/Water (700:300), 3.2 g Ammonium formate (=50 mM)

### Gradient:

| Time [min] | % A2 | % B2 | Flow [mL/min] | Profile |
|---|---|---|---|---|
| 0 | 98 | 2 | 0.5 | |
| 0.5 | 98 | 2 | 0.5 | 6 |
| 1.0 | 10 | 90 | 0.5 | 6 |
| 2.0 | 10 | 90 | 0.5 | 6 |
| 2.1 | 98 | 2 | 0.6 | 6 |

A2 = Water 0.01% Formic acid,
B2 = ACN/Water (700:300), 3.2 g Ammonium formate (=50 mM)
HPLC Column Biobasic SCX, 5 micron 2.1 × 50 mm, Thermo

### Target Needle Wash Procedure

Use Isopropanol with a target flush volume of 0.500 mL for strong solvent wash and water for the weak solvent wash post-wash.

### Compound Set 4 (1,5-Anhydroglucitol):

### Mass Spec Conditions for Compound Set 4 (1,5-Anhydroglucitol)

Source Type: HESI source
Monitor: Selected Reaction Monitoring (SRM), negative mode

### Chromatographic Conditions for Compound Set 4 (1,5-Anhydroglucitol)

### Mobile Phase A1 Water/Ammonium Bicarbonate, 500:1

### Mobile Phase B1 ACN/MeOH (1:1)

### Isocratic

| Time [min] | % A | % B | Flow [mL/min] | Profile |
|---|---|---|---|---|
| 0 | 15 | 85 | 0.5 | |

HPLC Column: Acquity C 18 BEH, 1.7 micron 2.1 × 100 mm, Waters

### Target Needle Wash Procedure

Use Isopropanol with a target flush volume of 0.500 mL for strong solvent wash and water for the weak solvent wash post-wash.

TABLE 16A

| | | Ion | | Ion |
|---|---|---|---|---|
| | Analyte Reference | Monitored/ | Internal Standard | Monitored/ |
| | Compound | Transition | Reference Compound | Transition |
| 1 | palmitate (16:0) | 255.3 | palmitic acid ¹³C₁₆ | 271.3 |
| | | ->255.3 | | ->271.3 |
| 2 | docosatetraenoic acid | 331.3 | palmitic acid ¹³C₁₆ | |
| | | ->331.3 | | |
| 3 | oleate (18:1(n-9)) + 1359 | 281.3 | oleic acid ¹³C₁₈ | 299.3 |
| | | ->281.3 | | ->299.3 |
| 4 | stearate (18:0) | 283.3 | octadecanoic acid- | 286.3 |
| | | ->283.3 | 18,18,18-D₃ | ->286.3 |
| 5 | margarate (17:0) | 269.3 | heptadecanoic acid- | 272.3 |
| | | ->269.3 | 17,17,17-D₃ | ->272.3 |
| 6 | linoleate (18:2(n-6)) | 277.3 | linoleic acid ¹³C₁₈ | 297.3 |
| | | ->277.3 | | -> |
| 7 | linolenate (18:2(n-6)) | 279.3 | linolenic acid ¹³C₁₈ | 295.3 |
| | | ->279.3 | | ->295.3 |
| 8 | pamitoleic acid | 253.2 | linolenic acid ¹³C₁₈ | |
| | | ->253.2 | | |
| 9 | linoleoyl-lyso-GPC | 520.6 | linoleoyl-lyso-GPC- | 529.6 |
| | | ->184.1 | (N,N,N-triMe-D₉) | ->193.1 |
| 10 | oleoyl-lyso-GPC | 522.6 | linoleoyl-lyso-GPC- | |
| | | ->184.1 | (N,N,N-triMe-D₉) | |
| 11 | palmitoyl-lyso-GPC | 496.6 | linoleoyl-lyso-GPC- | |
| | | ->184.1 | (N,N,N-triMe-D₉) | |
| 12 | octanoyl carnitine | 288.4 | octanoyl carnitine-(N- | 291.4 |
| | chloride | ->85.1 | methyl-D₃) HCl | ->85.1 |
| 13 | decanoyl carnitine | 316.4 | decanoyl carnitine-(N- | 319.4 |
| | chloride | ->85.1 | methyl-D₃) HCl | ->85.1 |
| 14 | 2-hydroxybutyrate | 103.1 | Na-2-hydroxybutyrate- | 106.1 |
| | | ->57.1 | 2,3,3-D₃ | ->59.1 |
| 15 | 3-methyl-2-oxobutyrate | 115.1 | 3-methyl-2oxobutyrate- | 122.1 |
| | | ->71.1 | D₇ | ->78.1 |
| 16 | 3-hydroxybutyrate | 103.1 | Na-3-hydroxybutyrate- | 107.1 |
| | | ->59.1 | 3,4,4,4-D₄ | ->59.1 |
| 17 | 1,5-anhydroglucitol | 163.1 | 1,5-anhydroglucitol-1,5-¹³C₆ | 169.1 |
| | | ->101.1 | | ->105.1 |
| 18 | creatine | 132.1 | creatine (Methyl)-D₃ | 135.1 |
| | | ->90.1 | | ->93.1 |
| 19 | serine | 106.1 | serine-2,3,3-D₃ | 109.1 |
| | | -60.1 | | ->63.1 |
| 20 | arginine | 175.1 | arginine-¹³C₆ | 181.1 |
| | | ->70.1 | | ->74.1 |
| 21 | glycine | 76.1 | glycine ¹³C₂-¹⁵N | 79.1 |
| | | ->30.1 | | ->32.1 |
| 22 | betaine | 118.1 | betaine-D₉ (N,N,N- | 127.1 |
| | | ->58.1 | trimethyl-D₉) | ->66.1 |
| 23 | glutamic acid | 148.1 | glutamic acid-2,3,3,4,4- | 153.1 |
| | | ->84.1 | D₅ | ->88.1 |
| 24 | threonine | 120 | threonine-¹³C₄-¹⁵N | 125 |
| | | ->74.1 | | ->78.1 |
| 25 | tryptophan | 205.2 | tryptophan-D₅ | 210.2 |
| | | ->146.1 | | ->151.1 |
| 26 | Gamma-glutamyl-leucine | 261.2 | betaine-D₉ (N,N,N- | 127.1 |
| | | ->132.1 | trimethyl-D₉) | ->66.1 |
| 27 | Glutamyl-valine | 247.2 | betaine-D₉ (N,N,N- | 127.1 |
| | | ->118.1 | trimethyl-D₉) | ->66.1 |
| 28 | Stearoyl-lyso-GPC | 524.6 | linoleoyl-lyso-GPC- | 529.6 |
| | | ->184.1 | (N,N,N-triMe-D₉) | ->193.1 |
| 29 | Cis-10-Heptadecenoic | 267.3 | palmitic acid ¹³C₁₆ | 271.3 |
| | acid | ->267.3 | | ->271.3 |

TABLE 16B
Calibration standard target concentrations

| | STD A, | STD B, | STD C, | STD D, | STD E, | STD F, |
|---|---|---|---|---|---|---|
| | Target | Target | Target | Target | Target | Target |
| | conc | conc | conc | conc | conc | conc |
| Reference Standard | (ug/mL) | (ug/mL) | (ug/mL) | (ug/mL) | (ug/mL) | (ug/mL) |
| palmitate (16:0) | 5.000 | 10.000 | 25.000 | 80.000 | 140.000 | 200.000 |
| docosatetraenoic acid | 0.050 | 0.100 | 0.250 | 0.800 | 1.400 | 2.000 |
| oleate (18:1(n-9)) + 1359 | 10.000 | 20.000 | 50.000 | 160.000 | 280.000 | 400.000 |
| stearate (18:0) | 2.500 | 5.000 | 12.500 | 40.000 | 70.000 | 100.000 |
| margarate (17:0) | 0.025 | 0.050 | 0.125 | 0.400 | 0.700 | 1.000 |
| linoleate (18:2(n-6)) | 2.500 | 5.000 | 12.500 | 40.000 | 70.000 | 100.000 |
| linolenate (18:2(n-6)) | 0.150 | 0.300 | 0.750 | 2.400 | 4.200 | 6.000 |
| pamitoleic acid | 1.000 | 2.000 | 5.000 | 16.000 | 28.000 | 40.000 |
| linoleoyl-lyso-GPC | 2.500 | 5.000 | 12.500 | 40.000 | 70.000 | 100.000 |
| oleoyl-lyso-GPC | 2.500 | 5.000 | 12.500 | 40.000 | 70.000 | 100.000 |
| palmitoyl-lyso-GPC | 2.500 | 5.000 | 12.500 | 40.000 | 70.000 | 100.000 |
| octanoyl carnitine | 0.003 | 0.006 | 0.015 | 0.048 | 0.084 | 0.120 |
| chloride | | | | | | |
| decanoyl carnitine | 0.003 | 0.006 | 0.015 | 0.048 | 0.084 | 0.120 |
| chloride | | | | | | |
| 2-hydroxybutyrate | 0.500 | 1.000 | 2.500 | 8.000 | 14.000 | 20.000 |
| 3-methyl-2-oxobutyrate | 0.500 | 1.000 | 2.500 | 8.000 | 14.000 | 20.000 |
| 3-hydroxybutyrate | 0.500 | 1.000 | 2.500 | 8.000 | 14.000 | 20.000 |
| 1,5-anhydroglucitol | 2.000 | 4.000 | 10.000 | 32.000 | 56.000 | 80.000 |
| creatine | 0.500 | 1.000 | 2.500 | 8.000 | 14.000 | 20.000 |
| Serine | 1.250 | 2.500 | 6.250 | 20.000 | 35.000 | 50.000 |
| arginine | 1.250 | 2.500 | 6.250 | 20.000 | 35.000 | 50.000 |
| glycine | 1.250 | 2.500 | 6.250 | 20.000 | 35.000 | 50.000 |
| betaine | 0.500 | 1.000 | 2.500 | 8.000 | 14.000 | 20.000 |
| glutamic acid | 1.000 | 2.000 | 5.000 | 16.000 | 28.000 | 40.000 |
| threonine | 1.250 | 2.500 | 6.250 | 20.000 | 35.000 | 50.000 |
| tryptophan | 0.400 | 0.800 | 2.000 | 6.400 | 11.200 | 16.000 |
| Gamma-glutamyl-leucine | 0.010 | 0.020 | 0.050 | 0.160 | 0.280 | 0.400 |
| Glutamyl-valine | 0.010 | 0.020 | 0.050 | 0.160 | 0.280 | 0.400 |
| Stearoyl-lyso-GPC | 2.500 | 5.000 | 12.500 | 40.000 | 70.000 | 100.000 |
| Cis-10-Heptadecenoic | 0.025 | 0.050 | 0.125 | 0.400 | 0.700 | 1.000 |
| acid | | | | | | |

### Example 9

### Using IR Biomarkers in Additional Statistical Analysis to Model IR and Evaluation of the Models in an Independent Cohort

Various statistical techniques (Bayesian elastic net, linear regression, logistic regression, etc.) were used to determine the insulin resistance status of a subject by either a continuous model or a classification model using the data from the targeted assays developed for biomarkers numbered 1-24 as listed in Table 16A. Variations of linear regression models were used to correlate baseline levels of the 24 individual biomarker compounds to the glucose disposal rate (Rd expressed as Mffm or Mwbm) as measured by the euglycemic hyperinsulinemic clamp for each individual. Models were generated using 399 non-diabetic subjects from the cohort described in Table 3.

Table 17 shows the additional models using the IR biomarkers to determine insulin resistance of a subject. For Tables 17A and 17B, the Biomarkers are listed in the first column and Model Names and Model Numbers are listed in the first and second row respectively. Data transformation was performed on certain biomarkers as indicated (e.g., squared, square root, etc.). Biomarkers separated by an * indicates the values for the markers were multiplied and the product obtained was used in the model with the indicated coefficient.

Three statistical methods were used to generate the continuous models for the prediction of Rd (Mwbm or Mffm) listed in Table 17A. One statistical method for generating a model for predicting Rd utilized a Bayesian elastic net method with a gamma prior assigned to one of the tuning parameters so that there is only one tuning parameter. A second statistical method used a combination of Multifactor Reduction (MDR) analysis (Ritchie et al., 2001 American Journal of Human Genetics 69:138-147) and Generalized Multifactor Dimensionality Reduction (GMDR) analysis (Lou et al., 2007 American Journal of Human Genetics 80: 1125-1137) to identify compounds and clinical covariates that predict insulin resistance or Rd. Following variable selection, least-square regression, minimizing least squares, using Statav11 (Davidson, R., and J. G. MacKinnon. 1993. Estimation and Inference in Econometrics. New York: Oxford University Press) was used to generate models for predicting Rd expressed as Mffm or Mwbm. Finally, multiple linear regression using a forward selection technique was utilized to generate additional continuous models.

Statistical analysis was performed to generate models to classify a subject as insulin resistant or insulin sensitive using various thresholds for separating IR individuals from IS individuals. Methods for classifying subjects as insulin resistant or insulin sensitive were generated using logistic regression based on optimizing the Area Under the Receiver Operating Characteristic (AUC) curve. Logistic regression is described in more detail in Example 3B above. Various cut-offs for Rd were modeled (Mffm: 37, 39, 45 umol/min/kg fat-free mass; Mwbm: 4, 4.5, 5.6 mg/kg/min) using logistic regression. Models utilizing this method are provided in Table 17B.

Another approach to classification of subjects is using Random Forest Analysis. Random forests create a set of classification trees based on continual sampling of the experimental units and compounds. Then each observation is classified based on the majority votes from all the classification trees. Models generated using this method are listed in Table 17B.

When fasting insulin is considered, there are 4 variables that stand out in the random forest analysis. Rather than having a complex forest, we fit the four individual trees using, for example “rpart” in the R-package. For IR defined as M_wbm<=5.6, the four trees are listed below.

(1) if BMI>=26.55, then IR

(2) if AHB>=5.0802, then IR

(3) if linoleoylGPC<15.60359, then IR

(4) if insulin>=35.925, then IR

Rather than computing the probability of IR, we can compute a risk-score: for each of the (4) conditions satisfied, one point is assigned (hence, possible scores are 0, 1, 2, 3, 4). For example, suppose a subject has BMI=25, AHB=5.2, linoleoylGPC=17, and insulin=36. Then the score is 0+1+1+1=3.

Using the cohort described in Table 5 the statistics for the training set were the following if a score of 2-4 is considered “positive”: sensitivity=83%, specificity=83%, PPV=74%, NPV=90%; and for the test set, the statistics were the following: Sensitivity=77%, Specificity=84%, PPV=75%, NPV=86%.

The clinical parameter, Fasting insulin, was included as a variable in some continuous models for predicting Rd and some classification models.

### Insulin Resistance Models.

TABLE 17A
Regression models to predict glucose disposal rate of an individual
as a continuous variable.

| | MODEL NAME | | | | | | |
|---|---|---|---|---|---|---|---|
| | AMR_ModelFFM_1 | AMR_ModelFFM_2 | AMR_ModelWBM_1 | AMR_ModelWBM_2 | CC_ModelWBM_1 | JL_ModelFFM_1 | JL_ModelFFM_2 |
| | MODEL NUMBER | | | | | | |
| | 1 | 2 | 3 | 4 | 5 | 6 | 7 |
| | RESPONSE | | | | | | |
| | Mffm | Mffm | Mwbm | Mwbm | sqrt(Mwbm) | Mffm | Mffm |
| Intercept | 89.62439 | 91.74204 | 13.19453 | 15.96521 | 4.46109969 | 55.685 | 57.4044 |
| docosatetraenoic_acid | | | | | | | −3.52981 |
| docosatetraenoic_acid_squared | | | | | | | 0.619029 |
| 2-hydroxybutyrate | −4.05776 | | −0.447938 | −0.5028735 | −0.0820386 | −6.19225 | −5.73734 |
| 2- | | | | | | 0.330463 | |
| hydroxybutyrate_squared | | | | | | | |
| betaine | | | | | | | |
| betaine_squared | | | | | | −1.11536 | −1.2503 |
| 3-hydroxy-butyrate | | | | | | | |
| BMI | −0.55264 | −1.987228 | −0.178318 | −0.3253534 | −0.0616969 | −1.7046 | −4.87323 |
| creatine | | | | | −0.0290852 | | |
| creatine_squared | | | | | | | −0.631571 |
| decanoyl_carnitine | 101.6699 | | 13.91962 | | 2.78281768 | 4.75504 | 5.19896 |
| glutamic_acid | | | | | | 0.661715 | |
| glycine | | | | | | 1.73791 | 2.62397 |
| INSULIN | −0.31398 | | −0.043469 | | | −9.01689 | |
| INSULIN_squared | | | | | | 1.54029 | |
| 3-methyl-2- | | | | | | −1.29966 | −1.98947 |
| oxobutyric_acid | | | | | | | |
| 3-methyl-2- | | | | | | | |
| oxobutyric_acid_squared | | | | | | | |
| linolenic_acid | | | | | | −0.970253 | −1.9241 |
| linoleoyl-LPC | 0.301117 | 0.9097335 | 0.098421 | 0.1249701 | 0.01632765 | 0.182954 | 1.84935 |
| margaric_acid | | | | | | | |
| margaric_acid_squared | | | | | | 0.143932 | |
| oleoyl-LPC | | | | | | | |
| palmitate | | | | | −0.0049244 | | |
| palmitoleic_acid | | | | | | | 2.29402 |
| stearate | | | | | | −1.13085 | |
| threonine | | | | | | −1.04512 | −1.42462 |
| tryptophan | | | | | | 0.386191 | |

| | MODEL NAME | | | | | | | |
|---|---|---|---|---|---|---|---|---|
| | JL_ModelFFM_3 | JL_ModelFFM_4 | JL_ModelWBM_1 | JL_ModelWBM_2 | JL_ModelWBM_3 | JL_ModelWBM_4 | MM_ModelFFM_1 | MM_ModelFFM_10 |
| | MODEL NUMBER | | | | | | | |
| | 8 | 9 | 10 | 11 | 12 | 13 | 14 | 15 |
| | RESPONSE | | | | | | | |
| | In(Mffm) | In(Mffm) | Mwbm | Mwbm | In(Mwbm) | In(Mwbm) | sqrt(Mffm) | sqrt(Mffm) |
| Intercept | 3.9289 | 3.9642 | 7.2517 | 7.4925 | 1.908 | 1.936 | 12.268826 | 10.9050161 |
| docosatetraenoic_acid | | −0.02345 | | −0.50871 | | −0.032521 | | |
| docosatetraenoic_acid_squared | | | | 0.099763 | | | | |
| 2-hydroxybutyrate | −0.1168 | −0.12153 | −0.6995 | −0.67914 | −0.10734 | −0.12323 | −0.3426834 | −0.2966596 |
| 2- | | | | | | | | |
| hydroxybutyrate_squared | | | | | | | | |
| betaine | | 0.003504 | 0.02138 | 0.051937 | 0.03671 | 0.045305 | | |
| betaine_squared | −0.02455 | −0.02764 | −0.1433 | −0.18102 | −0.035591 | −0.041583 | | |
| 3-hydroxy-butyrate | | | | 0.074189 | | | | |
| BMI | −0.03857 | −0.1119 | −0.7039 | −1.1378 | −0.121 | −0.19424 | −0.1317508 | −0.051362 |
| creatine | | | | −0.26454 | | | | |
| creatine_squared | | −0.01467 | | | | | | |
| decanoyl_carnitine | 0.081591 | 0.084283 | 0.64881 | 0.67063 | 0.088364 | 0.094242 | | |
| glutamic_acid | | | 0.1401 | 0.13431 | | | | |
| glycine | 0.021903 | 0.045677 | | 0.23273 | | 0.027763 | | |
| INSULIN | −0.18389 | | −1.4043 | | −0.19677 | | | −0.0239152 |
| INSULIN_squared | 0.028044 | | 0.28934 | | 0.027442 | | | |
| 3-methyl-2- | −0.01719 | −0.02849 | | −0.01193 | | | | |
| oxobutyric_acid | | | | | | | | |
| 3-methyl-2- | | 0.00315 | | | | | | |
| oxobutyric_acid_squared | | | | | | | | |
| linolenic_acid | −0.0219 | −0.02297 | −0.1758 | −0.22441 | −0.034311 | −0.034763 | | |
| linoleoyl-LPC | 0.013207 | 0.035984 | 0.27746 | 0.37802 | 0.03578 | 0.054311 | | |
| margaric_acid | | | | −0.013335 | | | | |
| margaric_acid_squared | 0.008648 | 0.010746 | 0.0866 | 0.041231 | | | | |
| oleoyl-LPC | | | | | | | | |
| palmitate | | | | | | | | |
| palmitoleic_acid | | 0.006985 | | 0.16019 | | | | |
| stearate | −0.04346 | −0.02384 | −0.2281 | −0.012588 | −0.051733 | −0.034267 | | |
| threonine | −0.00576 | −0.016 | −0.149 | −0.17098 | | | | |
| tryptophan | | | 0.25193 | 0.15482 | | | | |

| | MODEL NAME | | | | | | |
|---|---|---|---|---|---|---|---|
| | MM_ModelFFM_11 | MM_ModelFFM_2 | MM_ModelFFM_3 | MM_ModelFFM_4 | MM_ModelFFM_5 | MM_ModelFFM_6 | MM_ModelFFM_7 |
| | MODEL NUMBER | | | | | | |
| | 16 | 17 | 18 | 19 | 20 | 21 | 22 |
| | RESPONSE | | | | | | |
| | sqrt(Mffm) | sqrt(Mffm) | sqrt(Mffm) | sqrt(Mffm) | sqrt(Mffm) | sqrt(Mffm) | sqrt(Mffm) |
| Intercept | 10.1648049 | 11.6267767 | 11.0872046 | 11.2006603 | 11.3353881 | 9.84456196 | 9.40045333 |
| docosatetraenoic_acid | | | | | | | |
| docosatetraenoic_acid_squared | | | | | | | |
| 2-hydroxybutyrate | | −0.3459759 | −0.3104714 | −0.3116587 | −0.3244489 | −0.3076215 | −0.3117575 |
| 2- | | | | | | | |
| hydroxybutyrate_squared | | | | | | | |
| betaine | | | | | | | |
| betaine_squared | | | | | | | |
| 3-hydroxy-butyrate | | | | | | | |
| BMI | −0.0672296 | −0.1226056 | −0.1215338 | −0.1197936 | −0.1216324 | | |
| creatine | | | | | | | |
| creatine_squared | | | | | | | |
| decanoyl_carnitine | | 7.12037882 | | | | | 6.82789266 |
| glutamic_acid | | | | | | | |
| glycine | | | 0.02963783 | | | | |
| INSULIN | −0.0296831 | | | | | −0.0292776 | −0.0277471 |
| INSULIN_squared | | | | | | | |
| 3-methyl-2- | | | | | | | |
| oxobutyric_acid | | | | | | | |
| 3-methyl-2- | | | | | | | |
| oxobutyric_acid_squared | | | | | | | |
| linolenic_acid | | | | | | | |
| linoleoyl-LPC | | | | 0.03713148 | | | |
| margaric_acid | | | | | | | |
| margaric_acid_squared | | | | | | | |
| oleoyl-LPC | | | | | 0.05407326 | | |
| palmitate | | | | | | | |
| palmitoleic_acid | | | | | | | |
| stearate | | | | | | | |
| threonine | | | | | | | |
| tryptophan | | | | | | | |

| | MODEL NAME | | | | | | |
|---|---|---|---|---|---|---|---|
| | MM_ModelFFM_8 | MM_ModelFFM_9 | MM_ModelWBM_1 | MM_ModelWBM_10 | MM_ModelWBM_11 | MM_ModelWBM_12 | MM_ModelWBM_2 |
| | MODEL NUMBER | | | | | | |
| | 23 | 24 | 25 | 26 | 27 | 28 | 29 |
| | RESPONSE | | | | | | |
| | sqrt(Mffm) | sqrt(Mffm) | sqrt(Mwbm) | sqrt(Mwbm) | sqrt(Mwbm) | sqrt(Mwbm) | sqrt(Mwbm) |
| Intercept | 9.05083 | 9.08887957 | 4.18614118 | 4.27231483 | 4.00131844 | 4.21166889 | 4.3673563 |
| docosatetraenoic_acid | | | | | | | |
| docosatetraenoic_acid_squared | | | | | | | |
| 2-hydroxybutyrate | −0.282932 | −0.2800221 | −0.1028 | | −0.0986731 | −0.0963465 | −0.1001359 |
| 2- | | | | | | | |
| hydroxybutyrate_squared | | | | | | | |
| betaine | | | | | | | |
| betaine_squared | | | | | | | |
| 3-hydroxy-butyrate | | | | | | | |
| BMI | | | −0.0617679 | −0.0462348 | −0.0367478 | −0.0394254 | −0.064504 |
| creatine | | | | | | | |
| creatine_squared | | | | | | | |
| decanoyl_carnitine | | | 2.57266936 | | 2.54818097 | | |
| glutamic_acid | | | | | | | |
| glycine | 0.02379049 | | | | | | |
| INSULIN | −0.0276791 | −0.0273781 | | −0.0116565 | −0.0086304 | −0.0088871 | |
| INSULIN_squared | | | | | | | |
| 3-methyl-2- | | | | | | | |
| oxobutyric_acid | | | | | | | |
| 3-methyl-2- | | | | | | | |
| oxobutyric_acid_squared | | | | | | | |
| linolenic_acid | | | | | | | |
| linoleoyl-LPC | | 0.03352399 | 0.02195897 | | | | 0.02372363 |
| margaric_acid | | | | | | | |
| margaric_acid_squared | | | | | | | |
| oleoyl-LPC | | | | | 0.01988472 | 0.02065503 | |
| palmitate | | | | | | | |
| palmitoleic_acid | | | | | | | |
| stearate | | | | | | | |
| threonine | | | | | | | |
| tryptophan | | | | | | | |

| | MODEL NAME | | | | | | |
|---|---|---|---|---|---|---|---|
| | MM_ModelWBM_3 | MM_ModelWBM_4 | MM_ModelWBM_5 | MM_ModelWBM_6 | MM_ModelWBM_7 | MM_ModelWBM_8 | MM_ModelWBM_9 |
| | MODEL NUMBER | | | | | | |
| | 30 | 31 | 32 | 33 | 34 | 35 | 36 |
| | RESPONSE | | | | | | |
| | sqrt(Mwbm) | sqrt(Mwbm) | sqrt(Mwbm) | sqrt(Mwbm) | sqrt(Mwbm) | sqrt(Mwbm) | sqrt(Mwbm) |
| Intercept | 5.04981678 | 4.51856043 | 4.28726376 | 3.81218712 | 3.97378663 | 4.52614317 | 3.68389168 |
| docosatetraenoic_acid | | | | | | | |
| docosatetraenoic_acid_squared | | | | | | | |
| 2-hydroxybutyrate | −0.1199579 | −0.1095799 | −0.1110949 | −0.0892584 | −0.0858501 | −0.1017286 | −0.1104349 |
| 2- | | | | | | | |
| hydroxybutyrate_squared | | | | | | | |
| betaine | | | | | | | |
| betaine_squared | | | | | | | |
| 3-hydroxy-butyrate | | | | | | | |
| BMI | −0.0721435 | −0.0663848 | −0.0630448 | −0.0341624 | −0.0363543 | −0.0407936 | |
| creatine | | | | | | | |
| creatine_squared | | | | | | | |
| decanoyl_carnitine | | | 2.71298555 | 2.41158145 | | | |
| glutamic_acid | | | | | | | |
| glycine | | | | | | | |
| INSULIN | | | | −0.0087742 | −0.008984 | −0.0096786 | −0.0139376 |
| INSULIN_squared | | | | | | | |
| 3-methyl-2- | | | | | | | |
| oxobutyric_acid | | | | | | | |
| 3-methyl-2- | | | | | | | |
| oxobutyric_acid_squared | | | | | | | |
| linolenic_acid | | | | | | | |
| linoleoyl-LPC | | | | 0.01864053 | 0.02043783 | | |
| margaric_acid | | | | | | | |
| margaric_acid_squared | | | | | | | |
| oleoyl-LPC | | 0.03077522 | 0.03000273 | | | | |
| palmitate | | | | | | | |
| palmitoleic_acid | | | | | | | |
| stearate | | | | | | | |
| threonine | | | | | | | |
| tryptophan | | | | | | | |

The response is expressed as Mffm, Mwbm or a statistical transformation thereof; square root (sqrt), natural log (ln).

TABLE 17B
Logistic Regression Models Using Biomarkers to Classify Subjects
According to IR Status (IR vs. not IR)

| | MODEL ID | | | | | | |
|---|---|---|---|---|---|---|---|
| | CH_F1_1a | CH_F1_1b | MM_F1_1 | MM_F1_2 | MM_F1_3 | MM_F1_4 | MM_F1_5 |
| | MODEL NUMBER | | | | | | |
| | 1 | 2 | 3 | 4 | 5 | 6 | 7 |
| | RESPONSE | | | | | | |
| | F1 | F1 | F1 | F1 | F1 | F1 | F1 |
| Intercept | −3.9866 | −2.2501 | −5.3675921 | −5.84969 | −9.02499 | −5.3781082 | −2.8057909 |
| 2-hydroxybutyrate | 0.3942 | 0.4183 | 0.52426629 | 0.510859 | 0.596339 | 0.56836274 | 0.48482542 |
| arginine | | | | | | | |
| betaine | | | | | | | |
| 3-hydroxy-butyrate | | | | | | | |
| BMI | | 0.00331 | 0.17914699 | 0.18123 | 0.21492 | | |
| BMI*betaine | | | | | | | |
| BMI*linoleoyl-LPC | | | | | | | |
| BMI*octano_decano_mean | | 3.8875 | | | | | |
| BMI*palmitoleic_acid | | | | | | | |
| creatine | | | | | | | |
| decanoylcarnitine | | | −11.531752 | | | | |
| glycine | | | | | | | |
| glycine*arginine | | | | | | | |
| INSULIN | 0.052 | | | | | 0.05380396 | 0.04931527 |
| INSULIN*3-hydroxy-butyrate | | | | | | | |
| INSULIN*octano_decano_mean | | | | | | | |
| 3-methyl-2-oxo-butyric_acid | | 0.625 | | | | | |
| linolenic_acid | | | | | | | |
| linolenic_acid*2-hydroxybutyrate | | | | | | | |
| linolenic_acid*betaine | | | | | | | |
| linoleoyl-LPC | −0.1203 | −0.1139 | −0.108174 | −0.11824 | | | −0.126013 |
| linoleoyl-LPC*betaine | | | | | | | |
| linoleoyl-LPC*3-hydroxy-butyrate | | | | | | | |
| linoleoyl-LPC*INSULIN | | | | | | | |
| linoleoyl-LPC*stearate | | | | | | | |
| margaric_acid | −4.8892 | | | | | | |
| margaric_acid*betaine | | | | | | | |
| octano_decano_mean | −19.4418 | −121.9 | | | | | |
| palmitoleic_acid | | | | | | | |
| palmitoleic_acid*margaric_acid | | | | | | | |
| serine | | −0.2321 | | | | | |
| stearate | 0.2531 | 0.1216 | | | | | |
| stearate*margaric_acid | | | | | | | |
| threonine | 0.0609 | 0.1165 | | | | | |
| | MODEL ID | | | | | | |
| | MM_F1_6 | MM_F1_7 | CH_F2_1a | CH_F2_2b | MM_F2_1 | MM_F2_2 | MM_F2_3 |
| | MODEL NUMBER | | | | | | |
| | 8 | 9 | 10 | 11 | 12 | 13 | 14 |
| | RESPONSE | | | | | | |
| | F1 | F1 | F2 | F2 | F2 | F2 | F2 |
| Intercept | −4.310681 | −2.56114 | −2.5753 | 10.3068 | −6.4930068 | −4.1229685 | −8.6013797 |
| 2-hydroxybutyrate | 0.48253707 | 0.480687 | 0.5239 | 0.5626 | 0.66275029 | 0.58198593 | 0.65609987 |
| arginine | | | | −0.2103 | | | |
| betaine | | | | | | | |
| 3-hydroxy-butyrate | | | | | | | |
| BMI | 0.06298219 | | | −0.2181 | | | 0.09606189 |
| BMI*betaine | | | | | | | |
| BMI*linoleoyl-LPC | | | | 0.0205 | | | |
| BMI*octano_decano_mean | | | | | | | |
| BMI*palmitoleic_acid | | | | 0.019 | | | |
| creatine | | | −0.1068 | | | | |
| decanoylcarnitine | | −11.2485 | | | | | |
| glycine | | | | −0.2936 | | | |
| glycine*arginine | | | | 0.0134 | | | |
| INSULIN | 0.04345538 | 0.049571 | 0.00981 | | 0.05646087 | 0.05239769 | 0.04726506 |
| INSULIN*3-hydroxy-butyrate | | | | | | | |
| INSULIN*octano_decano_mean | | | 1.1473 | | | | |
| 3-methyl-2-oxo-butyric_acid | | | | | | | |
| linolenic_acid | | | | | | | |
| linolenic_acid*2-hydroxybutyrate | | | | | | | |
| linolenic_acid*betaine | | | | | | | |
| linoleoyl-LPC | −0.1186228 | −0.10633 | −0.1176 | −0.6746 | | −0.1162301 | |
| linoleoyl-LPC*betaine | | | | | | | |
| linoleoyl-LPC*3-hydroxy-butyrate | | | | | | | |
| linoleoyl-LPC*INSULIN | | | | | | | |
| linoleoyl-LPC*stearate | | | | | | | |
| margaric_acid | | | −6.6696 | −8.98 | | | |
| margaric_acid*betaine | | | | | | | |
| octano_decano_mean | | | −65.1986 | | | | |
| palmitoleic_acid | | | | −0.8981 | | | |
| palmitoleic_acid*margaric_acid | | | | 0.6441 | | | |
| serine | | | | | | | |
| stearate | | | 0.3347 | 0.2604 | | | |
| stearate*margaric_acid | | | | | | | |
| threonine | | | | | | | |
| | MODEL ID | | | | | | |
| | MM_F2_4 | MM_F2_5 | CH_F3_1a | CH_F3_1b | MM_F3_1 | MM_F3_2 | MM_F3_3 |
| | MODEL NUMBER | | | | | | |
| | 15 | 16 | 17 | 18 | 19 | 20 | 21 |
| | RESPONSE | | | | | | |
| | F2 | F2 | F3 | F3 | F3 | F3 | F3 |
| Intercept | −10.45933 | −7.58282 | −9.2676 | −8.2893 | −11.299202 | −8.8805797 | −6.6962908 |
| 2-hydroxybutyrate | 0.67427193 | 0.595445 | 0.4125 | 0.4698 | 0.61506793 | 0.54776883 | 0.60710423 |
| arginine | | | | | | | |
| betaine | | | | | | | |
| 3-hydroxy-butyrate | | | | | | | |
| BMI | 0.23673564 | 0.204352 | 0.1206 | 0.2491 | 0.26815076 | 0.23980918 | |
| BMI*betaine | | | | | | | |
| BMI*linoleoyl-LPC | | | | | | | |
| BMI*octano_decano_mean | | | | | | | |
| BMI*palmitoleic_acid | | | | | | | |
| creatine | | | | | | | |
| decanoylcarnitine | | | | | | | |
| glycine | | | | | | | |
| glycine*arginine | | | | | | | |
| INSULIN | | | 0.0517 | | | | 0.06104198 |
| INSULIN*3-hydroxy-butyrate | | | | | | | |
| INSULIN*octano_decano_mean | | | | | | | |
| 3-methyl-2-oxo-butyric_acid | | | | | | | |
| linolenic_acid | | | | | | | |
| linolenic_acid*2-hydroxybutyrate | | | | | | | |
| linolenic_acid*betaine | | | | | | | |
| linoleoyl-LPC | | −0.10435 | −0.0743 | −0.0773 | | −0.0854535 | |
| linoleoyl-LPC*betaine | | | | | | | |
| linoleoyl-LPC*3-hydroxy-butyrate | | | | | | | |
| linoleoyl-LPC*INSULIN | | | | | | | |
| linoleoyl-LPC*stearate | | | | | | | |
| margaric_acid | | | −5.1633 | −5.0704 | | | |
| margaric_acid*betaine | | | | | | | |
| octano_decano_mean | | | | | | | |
| palmitoleic_acid | | | | | | | |
| palmitoleic_acid*margaric_acid | | | | | | | |
| serine | | | | −0.2088 | | | |
| stearate | | | 0.2985 | 0.2765 | | | |
| stearate*margaric_acid | | | | | | | |
| threonine | | | | | | | |
| | MODEL ID | | | | | | |
| | MM_F3_4 | MM_F3_5 | CH_G1_1a | CH_G1_1b | MM_G1_1 | MM_G1_2 | MM_G1_3 |
| | MODEL NUMBER | | | | | | |
| | 22 | 23 | 24 | 25 | 26 | 27 | 28 |
| | RESPONSE | | | | | | |
| | F3 | F3 | G1 | G1 | G1 | G1 | G1 |
| Intercept | −9.4214564 | −4.55906 | −3.6099 | −5.23 | −7.4390979 | −10.854343 | −5.9995013 |
| 2-hydroxybutyrate | 0.60145585 | 0.531876 | 0.442 | 0.553 | 0.54100073 | 0.63201682 | 0.60368583 |
| arginine | | | | | | | |
| betaine | | | | | | | |
| 3-hydroxy-butyrate | | | | −0.3964 | | | |
| BMI | 0.1222978 | | | −0.0656 | 0.23952185 | 0.2751019 | |
| BMI*betaine | | | | | | | |
| BMI*linoleoyl-LPC | | | | 0.0214 | | | |
| BMI*octano_decano_mean | | | | | | | |
| BMI*palmitoleic_acid | | | | | | | |
| creatine | | | | | | | |
| decanoylcarnitine | | | | | | | |
| glycine | | | | | | | |
| glycine*arginine | | | | | | | |
| INSULIN | 0.04970901 | 0.057281 | 0.0264 | | | | 0.06335795 |
| INSULIN*3-hydroxy-butyrate | | | | | | | |
| INSULIN*octano_decano_mean | | | 0.8216 | | | | |
| 3-methyl-2-oxo-butyric_acid | | | | | | | |
| linolenic_acid | | | | | | | |
| linolenic_acid*2-hydroxybutyrate | | | | | | | |
| linolenic_acid*betaine | | | | | | | |
| linoleoyl-LPC | | −0.10405 | −0.1547 | −0.3599 | −0.1288931 | | |
| linoleoyl-LPC*betaine | | | | | | | |
| linoleoyl-LPC*3-hydroxy-butyrate | | | | 0.0216 | | | |
| linoleoyl-LPC*INSULIN | | | | | | | |
| linoleoyl-LPC*stearate | | | | −0.0361 | | | |
| margaric_acid | | | −5.0029 | | | | |
| margaric_acid*betaine | | | | | | | |
| octano_decano_mean | | | −52.481 | −16.5695 | | | |
| palmitoleic_acid | | | | | | | |
| palmitoleic_acid*margaric_acid | | | | | | | |
| serine | | | | | | | |
| stearate | | | 0.2656 | 0.7 | | | |
| stearate*margaric_acid | | | | | | | |
| threonine | | | 0.1074 | | | | |
| | MODEL ID | | | | | | |
| | MM_G1_4 | MM_G1_5 | MM_G1_6 | CH_G2_1a | CH_G2_1b | MM_G2_1 | MM_G2_2 |
| | MODEL NUMBER | | | | | | |
| | 29 | 30 | 31 | 32 | 33 | 34 | 35 |
| | RESPONSE | | | | | | |
| | G1 | G1 | G1 | G2 | G2 | G2 | G2 |
| Intercept | −3.0817511 | −5.9460831 | −2.59736 | −5.5878 | −0.0295 | −12.2404 | −10.833987 |
| 2-hydroxybutyrate | 0.51242623 | 0.5166196 | 0.522548 | 0.5091 | 0.6343 | 0.58826369 | 0.59086609 |
| arginine | | | | 0.0927 | 0.1077 | | |
| betaine | | | | −2.4562 | −3.714 | | −0.4798465 |
| 3-hydroxy-butyrate | | | | | | | |
| BMI | | 0.11702808 | | 0.2141 | −0.1102 | 0.32022727 | 0.34202616 |
| BMI*betaine | | | | | 0.1169 | | |
| BMI*linoleoyl-LPC | | | | | | | |
| BMI*octano_decano_mean | | | | | | | |
| BMI*palmitoleic_acid | | | | | | | |
| creatine | | | | | | | |
| decanoylcarnitine | | | −12.3211 | | | | |
| glycine | | | | | | | |
| glycine*arginine | | | | | | | |
| INSULIN | 0.05919099 | 0.04900529 | 0.05492 | −0.0256 | | | |
| INSULIN*3-hydroxy-butyrate | | | | | | | |
| INSULIN*octano_decano_mean | | | | 1.011 | | | |
| 3-methyl-2-oxo-butyric_acid | | | | | | | |
| linolenic_acid | | | | | | | |
| linolenic_acid*2-hydroxybutyrate | | | | | | | |
| linolenic_acid*betaine | | | | | | | |
| linoleoyl-LPC | −0.1472082 | −0.1339163 | −0.13778 | −0.1134 | | | |
| linoleoyl-LPC*betaine | | | | 0.0721 | | | |
| linoleoyl-LPC*3-hydroxy-butyrate | | | | | | | |
| linoleoyl-LPC*INSULIN | | | | 0.00209 | | | |
| linoleoyl-LPC*stearate | | | | −0.0273 | | | |
| margaric_acid | | | | −12.4068 | | | |
| margaric_acid*betaine | | | | 2.2251 | | | |
| octano_decano_mean | | | | −50.9728 | −9.2556 | | |
| palmitoleic_acid | | | | | | | |
| palmitoleic_acid*margaric_acid | | | | | | | |
| serine | | | | | | | |
| stearate | | | | 0.7595 | | | |
| stearate*margaric_acid | | | | −0.1333 | | | |
| threonine | | | | | | | |
| | MODEL ID | | | | | | |
| | MM_G2_3 | MM_G2_4 | CH_G3_1a | CH_G3_1b | MM_G3_1 | MM_G3_2 | MM_G3_3 |
| | MODEL NUMBER | | | | | | |
| | 36 | 37 | 38 | 39 | 40 | 41 | 42 |
| | RESPONSE | | | | | | |
| | G2 | G2 | G3 | G3 | G3 | G3 | G3 |
| Intercept | −8.7637346 | −7.07327 | −10.5372 | −13.7198 | −12.776889 | −8.8502466 | −6.89142 |
| 2-hydroxybutyrate | 0.49482462 | 0.476283 | 0.4622 | 0.0381 | 0.60760604 | 0.50449061 | 0.477012 |
| arginine | | | 0.1133 | | | | |
| betaine | | | | 0.7815 | | | |
| 3-hydroxy-butyrate | | | −0.2006 | −0.1356 | | | |
| BMI | 0.28311565 | 0.151508 | 0.1567 | 0.3524 | 0.31833093 | 0.27711696 | 0.155198 |
| BMI*betaine | | | | | | | |
| BMI*linoleoyl-LPC | | | | | | | |
| BMI*octano_decano_mean | | | | | | | |
| BMI*palmitoleic_acid | | | | | | | |
| creatine | | | | | | | |
| decanoylcarnitine | | | | | | | |
| glycine | | | | | | | |
| glycine*arginine | | | | | | | |
| INSULIN | | 0.049353 | 0.0203 | | | | 0.040869 |
| INSULIN*3-hydroxy-butyrate | | | 0.00391 | | | | |
| INSULIN*octano_decano_mean | | | | | | | |
| 3-methyl-2-oxo-butyric_acid | | | | | | | |
| linolenic_acid | | | | 0.7652 | | | |
| linolenic_acid*2-hydroxybutyrate | | | | 0.1839 | | | |
| linolenic_acid*betaine | | | | −0.3301 | | | |
| linoleoyl-LPC | −0.1307299 | −0.13819 | −0.1603 | −0.151 | | −0.152181 | −0.17247 |
| linoleoyl-LPC*betaine | | | | | | | |
| linoleoyl-LPC*3-hydroxy-butyrate | | | | | | | |
| linoleoyl-LPC*INSULIN | | | | | | | |
| linoleoyl-LPC*stearate | | | | | | | |
| margaric_acid | | | | −8.9156 | | | |
| margaric_acid*betaine | | | | | | | |
| octano_decano_mean | | | | | | | |
| palmitoleic_acid | | | | | | | |
| palmitoleic_acid*margaric_acid | | | | | | | |
| serine | | | | | | | |
| stearate | | | 0.212 | 0.3904 | | | |
| stearate*margaric_acid | | | | | | | |
| threonine | | | | | | | |

TABLE 17C
Random Forest Classification of Subjects According to IR Status
Using IR Biomarkers for Risk Score Determination

| Model | Model | |
|---|---|---|
| No. | Name | Variables Considered |
| 1 | RFG1_1 | all 24 IR Biomarker metabolites |
| 2 | RFG1_2 | BMI, 2-hydroxybutyrate, Linoleoyl-LPC |
| 3 | RFG2_1 | BMI, 2-hydroxybutyrate, Linoleoyl-LPC |
| 4 | RFG3_1 | BMI, 2-hydroxybutyrate, Linoleoyl-LPC |
| 5 | RFG1_3 | Insulin, BMI, 2-hydroxybutyrate, Linoleoyl-LPC |
| 6 | RFG2_2 | Insulin, BMI, 2-hydroxybutyrate, Linoleoyl-LPC |
| 7 | RFG3_2 | Insulin, BMI, 2-hydroxybutyrate, Linoleoyl-LPC |
| 8 | RFF1_1 | BMI, 2-hydroxybutyrate, Linoleoyl-LPC |
| 9 | RFF1_2 | BMI, 2-hydroxybutyrate, Linoleoyl-LPC, |
| | | glycine |
| 10 | RFF1_3 | Insulin, 2-hydroxybutyrate, Linoleoyl-LPC |
| 11 | RFF1_4 | Insulin, 2-hydroxybutyrate, Linoleoyl-LPC, BMI |
| 12 | RFF1_5 | Insulin, 2-hydroxybutyrate, Linoleoyl-LPC, BMI, |
| | | glycine |
| 13 | RFF2_1 | BMI, 2-hydroxybutyrate, Linoleoyl-LPC |
| 14 | RFF2_2 | BMI, 2-hydroxybutyrate, Linoleoyl-LPC, |
| | | glycine |
| 15 | RFF2_3 | Insulin, 2-hydroxybutyrate, Linoleoyl-LPC |
| 16 | RFF2_4 | Insulin, 2-hydroxybutyrate, Linoleoyl-LPC, BMI |
| 17 | RFF2_5 | Insulin, 2-hydroxybutyrate, Linoleoyl-LPC, BMI, |
| | | glycine |
| 18 | RFF3_1 | BMI, 2-hydroxybutyrate, Linoleoyl-LPC |
| 19 | RFF3_2 | BMI, 2-hydroxybutyrate, Linoleoyl-LPC, |
| | | glycine |
| 20 | RFF3_3 | Insulin, 2-hydroxybutyrate, Linoleoyl-LPC |
| 21 | RFF3_4 | Insulin, 2-hydroxybutyrate, Linoleoyl-LPC, BMI |
| 22 | RFF3_5 | Insulin, 2-hydroxybutyrate, Linoleoyl-LPC, BMI, |
| | | glycine |

Risk Score Models only applied to G1 (IR defined as M_wbm <=5.6)
RS1
BMI >=26.55
2-hydroxybutyrate >=5.08021
Linoleoyl-LPGC <15.60359
insulin >=35.925
One point is assigned to each condition satisfied (thus, 0-4 are the possible scores)

Each model was evaluated for performance by comparing the predicted Rd to the actual Rd value as measured by the euglycemic hyperinsulinemic clamp. Table 18A provides a summary of the performance for each continuous model using the Rsquare metric, and Table 18B provides for the classification models the summary of performance includes the area under the curve (AUC), specificity, sensitivity, positive predictive value (PPV) and negative predictive value (NPV).

TABLE 18A
Regression models to predict glucose disposal rate of an individual
as a continuous variable.

| # | MODEL NAME | RESPONSE | Rsq1 | Rsq2 | TERMS |
|---|---|---|---|---|---|
| 1 | CC_ModelMWBM_1 | rootMwbm | 0.48 | 0.51 | BMI, AHB, |
| | | | | | decanoylcarnitine, |
| | | | | | linoleoylGPC, creatine, |
| | | | | | palmitate |
| 2 | MM_ModelMWBM_1 | rootMwbm | 0.47 | 0.49 | BMI, AHB, |
| | | | | | decanoylcarnitine, |
| | | | | | linoleoylGPC |
| 3 | MM_ModelMWBM_1a | rootMwbm | 0.47 | 0.50 | BMI, AHB, |
| | | | | | decanoylcarnitine, |
| | | | | | linoleoylGPC, creatine |
| 4 | MM_ModelMWBM_2 | rootMwbm | 0.43 | 0.46 | BMI, AHB, linoleoylGPC |
| 5 | MM_ModelMWBM_3 | rootMwbm | 0.40 | 0.43 | BMI, AHB |
| 6 | MM_ModelMWBM_4 | rootMwbm | 0.42 | 0.45 | BMI, AHB, oleoylGPC |
| 7 | MM_ModelMWBM_5 | rootMwbm | 0.46 | 0.49 | BMI, AHB, |
| | | | | | decanoylcarnitine, |
| | | | | | oleoylGPC |
| 8 | MM_ModelMWBM_5a | rootMwbm | 0.47 | 0.50 | BMI, AHB, |
| | | | | | decanoylcarnitine, |
| | | | | | oleoylGPC, creatine |
| 9 | AMR_ModelWBM2 | Mwbm | 0.43 | | BMI, AHB, linoleoylGPC |
| 10 | JL_ModelWBM_2 | Mwbm | 0.52 | | BMI, AHB, |
| | | | | | decanoylcarnitine, |
| | | | | | adrenate, linoleoylGPC, |
| | | | | | creatine, glycine, |
| | | | | | linolenate, betaine\hat{ }2, |
| | | | | | threonine, palmitoleate, |
| | | | | | tryptophan, glutamate, |
| | | | | | adrenate, BHB, |
| | | | | | margarate\hat{ }2, margarate, |
| | | | | | stearate, ketovaline |
| 11 | JL_ModelWBM_4 | In(Mwbm) | 0.47 | 0.54 | BMI, AHB, |
| | | | | | decanoylcarnitine, |
| | | | | | linoleoylGPC, betaine, |
| | | | | | betaine\hat{ }2, linolenate, |
| | | | | | stearate, adrenate, glycine |
| 12 | MM_ModelMWBM_6 | rootMwbm | 0.51 | 0.53 | insulin, BMI, AHB, |
| | | | | | decanoylcarnitine, |
| | | | | | linoleoylGPC |
| 13 | MM_ModelMWBM_7 | rootMwbm | 0.48 | 0.50 | insulin, BMI, AHB, |
| | | | | | linoleoylGPC |
| 14 | MM_ModelMWBM_8 | rootMwbm | 0.46 | 0.48 | insulin, BMI, AHB |
| 15 | MM_ModelMWBM_9 | rootMwbm | 0.42 | 0.43 | insulin, AHB |
| 16 | MM_ModelMWBM_10 | rootMwbm | 0.39 | 0.41 | insulin, BMI |
| 17 | MM_ModelMWBM_11 | rootMwbm | 0.51 | 0.52 | insulin, BMI, AHB, |
| | | | | | decanoylcarnitine, |
| | | | | | oleoylGPC |
| 18 | MM_ModelMWBM_12 | rootMwbm | 0.47 | 0.49 | insulin, BMI, AHB, |
| | | | | | oleoylGPC |
| 19 | MM_ModelMWBM_13 | rootMwbm | 0.52 | 0.54 | insulin, BMI, AHB, |
| | | | | | decanoylcarnitine, |
| | | | | | linoleoylGPC, linolenate |
| 20 | MM_ModelMWBM_14 | rootMwbm | 0.51 | 0.54 | insulin, BMI, AHB, |
| | | | | | decanoylcarnitine, |
| | | | | | oleoylGPC, linolenate |
| 21 | AMR_ModelWBM1 | Mwbm | 0.50 | | insulin, BMI, AHB, |
| | | | | | decanoylcarnitine, |
| | | | | | linoleoylGPC |
| 22 | JL_ModelWBM_1 | Mwbm | 0.56 | | insulin, BMI, AHB, |
| | | | | | decanoylcarnitine, |
| | | | | | insulin\hat{ }2, linoleoylGPC, |
| | | | | | tryptophan, stearate, |
| | | | | | linolenate, threonine, |
| | | | | | betaine\hat{ }2, glutamate, |
| | | | | | margarate\hat{ }2, betaine |
| 23 | JL_ModelWBM_3 | In(Mwbm) | 0.52 | 0.59 | insulin, BMI, AHB, |
| | | | | | decanoylcarnitine, |
| | | | | | stearate, betaine, |
| | | | | | linoleoylGPC, betaine\hat{ }2, |
| | | | | | linolenate, insulin\hat{ }2 |
| 24 | MM_ModelMFFM_1 | rootMffm | 0.31 | 0.33 | BMI, AHB |
| 25 | MM_ModelMFFM_2 | rootMffm | 0.35 | 0.37 | BMI, AHB, |
| | | | | | decanoylcarnitine |
| 26 | MM_ModelMFFM_3 | rootMffm | 0.32 | 0.35 | BMI, AHB, glycine |
| 27 | MM_ModelMFFM_4 | rootMffm | 0.32 | 0.34 | BMI, AHB, linoleoylGPC |
| 28 | MM_ModelMFFM_5 | rootMffm | 0.32 | 0.34 | BMI, AHB, oleoylGPC |
| 29 | AMR_ModelFFM1 | Mffm | 0.40 | | BMI, AHB, |
| | | | | | decanoylcarnitine, insulin, |
| | | | | | linoleoylGPC |
| 30 | AMR_ModelFFM2 | Mffm | 0.22 | | BMI, linoleoylGPC |
| 31 | JL_ModelFFM_2 | Mffm | 0.43 | | AHB, decanoylcarnitine, |
| | | | | | BMI, adrenate, glycine, |
| | | | | | palmitoleate, ketovaline, |
| | | | | | linolenate, linoleoylGPC, |
| | | | | | threonine, betaine\hat{ }2, |
| | | | | | creatine\hat{ }2, adrenate\hat{ }2 |
| 32 | JL_ModelFFM_4 | In(Mffm) | 0.41 | 0.45 | AHB, BMI, |
| | | | | | decanoylcarnitine, glycine, |
| | | | | | linoleoylGPC, ketovaline, |
| | | | | | betaine\hat{ }2, stearate, |
| | | | | | adrenate, linolenate, |
| | | | | | threonine, creatine\hat{ }2, |
| | | | | | margarate\hat{ }2, |
| | | | | | palmitoleate, betaine, |
| | | | | | ketovaline\hat{ }2 |
| 33 | MM_ModelMFFM_6 | rootMffm | 0.36 | 0.37 | insulin, AHB |
| 34 | MM_ModelMFFM_7 | rootMffm | 0.40 | 0.41 | insulin, AHB, |
| | | | | | decanoylcarnitine |
| 35 | MM_ModelMFFM_8 | rootMffm | 0.37 | 0.38 | insulin, AHB, glycine |
| 36 | MM_ModelMFFM_9 | rootMffm | 0.36 | 0.38 | insulin, AHB, linoleoylGPC |
| 37 | MM_ModelMFFM_10 | rootMffm | 0.36 | 0.39 | insulin, BMI, AHB |
| 38 | MM_ModelMFFM_11 | rootMffm | 0.27 | 0.29 | insulin, BMI |
| 39 | JL_ModelFFM_1 | Mffm | 0.45 | | insulin, AHB, |
| | | | | | decanoylcarnitine, glycine, |
| | | | | | BMI, insulin\hat{ }2, ketovaline, |
| | | | | | stearate, betaine\hat{ }2, |
| | | | | | threonine, linolenate, |
| | | | | | glutamate, tryptophan, |
| | | | | | AHB\hat{ }2, linoleoylGPC, |
| | | | | | margarate |
| 40 | JL_ModelFFM_3 | In(Mffm) | 0.43 | 0.48 | insulin, AHB, |
| | | | | | decanoylcarnitine, |
| | | | | | stearate, BMI, insulin\hat{ }2, |
| | | | | | betaine\hat{ }2, glycine, |
| | | | | | linolenate, ketovaline, |
| | | | | | linoleoylGPC, |
| | | | | | margarate\hat{ }2, threonine |

The response is expressed as Mffm, Mwbm or a statistical transformation thereof;
square root (sqrt),
natural log (ln).
Rsq1 = R-squared on the untransformed data;
Rsq2 = R-squared on the transformed data.
\hat{ }2 indicates the term was squared.

TABLE 18B
Logistic Regression and Random Forest Models Using Biomarkers to
Classify Subjects According to IR Status (IR vs. not IR)

| | MODEL | | | CUT | | | | | | |
|---|---|---|---|---|---|---|---|---|---|---|
| # | NAME | RESPONSE¹ | TYPE | OFF | SENS | SPEC | PPV | NPV | AUC | TERMS² |
| 1 | CH_G1_1a | G1 | logistic | 0.3 | 0.86 | 0.77 | 0.65 | 0.92 | 0.89 | |
| | | | regression | | | | | | | |
| 2 | CH_G1_1b | G1 | logistic | 0.3 | 0.82 | 0.80 | 0.66 | 0.90 | 0.90 | |
| | | | regression | | | | | | | |
| 3 | MM_G1_1 | G1 | logistic | 0.3 | 0.80 | 0.76 | 0.62 | 0.89 | 0.86 | BMI, |
| | | | regression | | | | | | | AHB, |
| | | | | | | | | | | linoleoyl |
| | | | | | | | | | | GPC |
| 4 | MM_G1_2 | G1 | logistic | 0.3 | 0.78 | 0.74 | 0.60 | 0.87 | 0.85 | BMI, |
| | | | regression | | | | | | | AHB |
| 5 | MM_G1_3 | G1 | logistic | 0.3 | 0.76 | 0.77 | 0.61 | 0.87 | 0.86 | insulin, |
| | | | regression | | | | | | | AHB |
| 6 | MM_G1_4 | G1 | logistic | 0.3 | 0.80 | 0.78 | 0.63 | 0.89 | 0.88 | insulin, |
| | | | regression | | | | | | | AHB, |
| | | | | | | | | | | linoleoyl |
| | | | | | | | | | | GPC |
| 7 | MM_G1_5 | G1 | logistic | 0.3 | 0.81 | 0.79 | 0.64 | 0.90 | 0.89 | insulin, |
| | | | regression | | | | | | | AHB, |
| | | | | | | | | | | linoleoyl |
| | | | | | | | | | | GPC, |
| | | | | | | | | | | BMI |
| 8 | MM_G1_6 | G1 | logistic | 0.3 | 0.82 | 0.80 | 0.66 | 0.91 | 0.89 | insulin, |
| | | | regression | | | | | | | AHB, |
| | | | | | | | | | | linoleoyl |
| | | | | | | | | | | GPC, |
| | | | | | | | | | | decanoyl |
| | | | | | | | | | | carnitine |
| 9 | CH_G1_1a | G1 | logistic | 0.5 | 0.66 | 0.90 | 0.77 | 0.84 | 0.89 | |
| | | | regression | | | | | | | |
| 10 | CH_G1_1b | G1 | logistic | 0.5 | 0.73 | 0.92 | 0.80 | 0.88 | 0.90 | |
| | | | regression | | | | | | | |
| 11 | MM_G1_1 | G1 | logistic | 0.5 | 0.59 | 0.89 | 0.72 | 0.81 | 0.86 | BMI, |
| | | | regression | | | | | | | AHB, |
| | | | | | | | | | | linoleoyl |
| | | | | | | | | | | GPC |
| 12 | MM_G1_2 | G1 | logistic | 0.5 | 0.57 | 0.90 | 0.74 | 0.81 | 0.85 | BMI, |
| | | | regression | | | | | | | AHB |
| 13 | MM_G1_3 | G1 | logistic | 0.5 | 0.61 | 0.93 | 0.80 | 0.84 | 0.86 | insulin, |
| | | | regression | | | | | | | AHB |
| 14 | MM_G1_4 | G1 | logistic | 0.5 | 0.63 | 0.91 | 0.76 | 0.84 | 0.88 | insulin, |
| | | | regression | | | | | | | AHB, |
| | | | | | | | | | | linoleoyl |
| | | | | | | | | | | GPC |
| 15 | MM_G1_5 | G1 | logistic | 0.5 | 0.64 | 0.91 | 0.77 | 0.84 | 0.89 | insulin, |
| | | | regression | | | | | | | AHB, |
| | | | | | | | | | | linoleoyl |
| | | | | | | | | | | GPC, |
| | | | | | | | | | | BMI |
| 16 | MM_G1_6 | G1 | logistic | 0.5 | 0.65 | 0.91 | 0.77 | 0.84 | 0.89 | insulin, |
| | | | regression | | | | | | | AHB, |
| | | | | | | | | | | linoleoyl |
| | | | | | | | | | | GPC, |
| | | | | | | | | | | decanoyl |
| | | | | | | | | | | carnitine |
| 17 | RF_G1_1 | G1 | random | | 0.75 | 0.80 | 0.65 | 0.87 | 0.86 | all 24 |
| | | | forest | | | | | | | metabolites |
| 18 | RF_G1_2 | G1 | random | | 0.77 | 0.76 | 0.61 | 0.87 | 0.84 | BMI, 2- |
| | | | forest | | | | | | | hydroxy |
| | | | | | | | | | | butyrate, |
| | | | | | | | | | | Linoleoyl- |
| | | | | | | | | | | LPC |
| 19 | RF_G1_3 | G1 | random | | 0.77 | 0.77 | 0.61 | 0.87 | 0.86 | Insulin, |
| | | | forest | | | | | | | BMI, 2- |
| | | | | | | | | | | hydroxy |
| | | | | | | | | | | butyrate, |
| | | | | | | | | | | Linoleoyl- |
| | | | | | | | | | | LPC |
| 20 | RS1 | G1 | risk score³ | | 0.84 | 0.76 | 0.62 | 0.91 | 0.88 | Insulin, BMI, |
| | | | | | | | | | | 2- |
| | | | | | | | | | | hydroxy |
| | | | | | | | | | | butyrate, |
| | | | | | | | | | | Linoleoyl- |
| | | | | | | | | | | LPC |
| 21 | CH_G2_1a | G2 | logistic | 0.3 | 0.79 | 0.83 | 0.64 | 0.91 | 0.90 | |
| | | | regression | | | | | | | |
| 22 | CH_G2_1b | G2 | logistic | 0.3 | 0.83 | 0.87 | 0.69 | 0.94 | 0.94 | |
| | | | regression | | | | | | | |
| 23 | MM_G2_1 | G2 | logistic | 0.3 | 0.76 | 0.82 | 0.62 | 0.90 | 0.86 | BMI, |
| | | | regression | | | | | | | AHB |
| 24 | MM_G2_2 | G2 | logistic | 0.3 | 0.75 | 0.82 | 0.62 | 0.89 | 0.88 | BMI, |
| | | | regression | | | | | | | AHB, |
| | | | | | | | | | | betaine |
| 25 | MM_G2_3 | G2 | logistic | 0.3 | 0.77 | 0.80 | 0.60 | 0.90 | 0.88 | BMI, |
| | | | regression | | | | | | | AHB, |
| | | | | | | | | | | linoleoyl |
| | | | | | | | | | | GPC |
| 26 | MM_G2_4 | G2 | logistic | 0.3 | 0.79 | 0.85 | 0.66 | 0.92 | 0.91 | insulin, |
| | | | regression | | | | | | | BMI, |
| | | | | | | | | | | AHB, |
| | | | | | | | | | | linoleoyl |
| | | | | | | | | | | GPC |
| 27 | CH_G2_1a | G2 | logistic | 0.5 | 0.65 | 0.93 | 0.77 | 0.87 | 0.90 | |
| | | | regression | | | | | | | |
| 28 | CH_G2_1b | G2 | logistic | 0.5 | 0.65 | 0.93 | 0.78 | 0.88 | 0.94 | |
| | | | regression | | | | | | | |
| 29 | MM_G2_1 | G2 | logistic | 0.5 | 0.55 | 0.93 | 0.75 | 0.84 | 0.86 | BMI, |
| | | | regression | | | | | | | AHB |
| 30 | MM_G2_2 | G2 | logistic | 0.5 | 0.57 | 0.92 | 0.74 | 0.85 | 0.88 | BMI, |
| | | | regression | | | | | | | AHB, |
| | | | | | | | | | | betaine |
| 31 | MM_G2_3 | G2 | logistic | 0.5 | 0.53 | 0.92 | 0.72 | 0.84 | 0.88 | BMI, |
| | | | regression | | | | | | | AHB, |
| | | | | | | | | | | linoleoyl |
| | | | | | | | | | | GPC |
| 32 | MM_G2_4 | G2 | logistic | 0.5 | 0.59 | 0.93 | 0.74 | 0.86 | 0.91 | insulin, |
| | | | regression | | | | | | | BMI, |
| | | | | | | | | | | AHB, |
| | | | | | | | | | | linoleoyl |
| | | | | | | | | | | GPC |
| 33 | RF_G2_1 | G2 | random | | 0.81 | 0.75 | 0.56 | 0.91 | 0.84 | BMI, 2- |
| | | | forest | | | | | | | hydroxy |
| | | | | | | | | | | butyrate, |
| | | | | | | | | | | Linoleoyl- |
| | | | | | | | | | | LPC |
| 34 | RF_G2_2 | G2 | random | | 0.82 | 0.78 | 0.57 | 0.92 | 0.87 | Insulin, |
| | | | forest | | | | | | | BMI, 2- |
| | | | | | | | | | | hydroxy |
| | | | | | | | | | | butyrate, |
| | | | | | | | | | | Linoleoyl- |
| | | | | | | | | | | LPC |
| 35 | CH_G3_1a | G3 | logistic | 0.3 | 0.81 | 0.90 | 0.70 | 0.94 | 0.93 | |
| | | | regression | | | | | | | |
| 36 | CH_G3_1b | G3 | logistic | 0.3 | 0.75 | 0.91 | 0.69 | 0.93 | 0.93 | |
| | | | regression | | | | | | | |
| 37 | MM_G3_1 | G3 | logistic | 0.3 | 0.68 | 0.86 | 0.59 | 0.91 | 0.87 | BMI, |
| | | | regression | | | | | | | AHB |
| 38 | MM_G3_2 | G3 | logistic | 0.3 | 0.69 | 0.86 | 0.59 | 0.91 | 0.89 | BMI, |
| | | | regression | | | | | | | AHB, |
| | | | | | | | | | | linoleoyl |
| | | | | | | | | | | GPC |
| 39 | MM_G3_3 | G3 | logistic | 0.3 | 0.73 | 0.88 | 0.62 | 0.92 | 0.91 | insulin, |
| | | | regression | | | | | | | BMI, |
| | | | | | | | | | | AHB, |
| | | | | | | | | | | linoleoyl |
| | | | | | | | | | | GPC |
| 40 | CH_G3_1a | G3 | logistic | 0.5 | 0.68 | 0.95 | 0.81 | 0.91 | 0.93 | |
| | | | regression | | | | | | | |
| 41 | CH_G3_1b | G3 | logistic | 0.5 | 0.64 | 0.96 | 0.80 | 0.91 | 0.93 | |
| | | | regression | | | | | | | |
| 42 | MM_G3_1 | G3 | logistic | 0.5 | 0.49 | 0.95 | 0.75 | 0.87 | 0.87 | BMI, |
| | | | regression | | | | | | | AHB |
| 43 | MM_G3_2 | G3 | logistic | 0.5 | 0.49 | 0.94 | 0.68 | 0.87 | 0.89 | BMI, |
| | | | regression | | | | | | | AHB, |
| | | | | | | | | | | linoleoyl |
| | | | | | | | | | | GPC |
| 44 | MM_G3_3 | G3 | logistic | 0.5 | 0.53 | 0.94 | 0.72 | 0.88 | 0.91 | insulin, |
| | | | regression | | | | | | | BMI, |
| | | | | | | | | | | AHB, |
| | | | | | | | | | | linoleoyl |
| | | | | | | | | | | GPC |
| 45 | RF_G3_1 | G3 | random | | 0.78 | 0.74 | 0.46 | 0.92 | 0.84 | BMI, 2- |
| | | | forest | | | | | | | hydroxy |
| | | | | | | | | | | butyrate, |
| | | | | | | | | | | Linoleoyl- |
| | | | | | | | | | | LPC |
| 46 | RF_G3_2 | G3 | random | | 0.82 | 0.78 | 0.57 | 0.92 | 0.87 | Insulin, |
| | | | forest | | | | | | | BMI, 2- |
| | | | | | | | | | | hydroxy |
| | | | | | | | | | | butyrate, |
| | | | | | | | | | | Linoleoyl- |
| | | | | | | | | | | LPC |
| 47 | CH_F1_1a | F1 | logistic | 0.3 | 0.86 | 0.72 | 0.60 | 0.91 | 0.87 | |
| | | | regression | | | | | | | |
| 48 | CH_F1_1b | F1 | logistic | 0.3 | 0.81 | 0.78 | 0.64 | 0.90 | 0.88 | |
| | | | regression | | | | | | | |
| 49 | MM_F1_1 | F1 | logistic | 0.3 | 0.78 | 0.74 | 0.60 | 0.87 | 0.84 | AHB, |
| | | | regression | | | | | | | linoleoyl |
| | | | | | | | | | | GPC, |
| | | | | | | | | | | BMI, |
| | | | | | | | | | | decanoyl |
| | | | | | | | | | | carnitine |
| 50 | MM_F1_2 | F1 | logistic | 0.3 | 0.79 | 0.73 | 0.60 | 0.87 | 0.83 | AHB, |
| | | | regression | | | | | | | linoleoyl |
| | | | | | | | | | | GPC, |
| | | | | | | | | | | BMI |
| 51 | MM_F1_3 | F1 | logistic | 0.3 | 0.76 | 0.70 | 0.56 | 0.85 | 0.81 | AHB, |
| | | | regression | | | | | | | BMI |
| 52 | MM_F1_4 | F1 | logistic | 0.3 | 0.73 | 0.74 | 0.58 | 0.86 | 0.83 | AHB, |
| | | | regression | | | | | | | insulin |
| 53 | MM_F1_5 | F1 | logistic | 0.3 | 0.77 | 0.75 | 0.59 | 0.87 | 0.85 | AHB, |
| | | | regression | | | | | | | insulin, |
| | | | | | | | | | | linoleoyl |
| | | | | | | | | | | GPC |
| 54 | MM_F1_6 | F1 | logistic | 0.3 | 0.79 | 0.76 | 0.61 | 0.88 | 0.85 | insulin, |
| | | | regression | | | | | | | AHB, |
| | | | | | | | | | | linoleoyl |
| | | | | | | | | | | GPC, |
| | | | | | | | | | | BMI |
| 55 | MM_F1_7 | F1 | logistic | 0.3 | 0.78 | 0.77 | 0.62 | 0.88 | 0.86 | insulin, |
| | | | regression | | | | | | | AHB, |
| | | | | | | | | | | linoleoyl |
| | | | | | | | | | | GPC, |
| | | | | | | | | | | decanoyl |
| | | | | | | | | | | carnitine |
| 56 | CH_F1_1a | F1 | logistic | 0.5 | 0.71 | 0.89 | 0.76 | 0.86 | 0.87 | |
| | | | regression | | | | | | | |
| 57 | CH_F1_1b | F1 | logistic | 0.5 | 0.69 | 0.89 | 0.75 | 0.86 | 0.88 | |
| | | | regression | | | | | | | |
| 58 | MM_F1_1 | F1 | logistic | 0.5 | 0.55 | 0.88 | 0.70 | 0.80 | 0.84 | AHB, |
| | | | regression | | | | | | | linoleoyl |
| | | | | | | | | | | GPC, |
| | | | | | | | | | | BMI, |
| | | | | | | | | | | decanoyl |
| | | | | | | | | | | carnitine |
| 59 | MM_F1_2 | F1 | logistic | 0.5 | 0.53 | 0.89 | 0.71 | 0.79 | 0.83 | AHB, |
| | | | regression | | | | | | | linoleoyl |
| | | | | | | | | | | GPC, |
| | | | | | | | | | | BMI |
| 60 | MM_F1_3 | F1 | logistic | 0.5 | 0.50 | 0.89 | 0.71 | 0.78 | 0.81 | AHB, |
| | | | regression | | | | | | | BMI |
| 61 | MM_F1_4 | F1 | logistic | 0.5 | 0.58 | 0.93 | 0.79 | 0.82 | 0.83 | AHB, |
| | | | regression | | | | | | | insulin |
| 62 | MM_F1_5 | F1 | logistic | 0.5 | 0.60 | 0.91 | 0.76 | 0.83 | 0.85 | AHB, |
| | | | regression | | | | | | | insulin, |
| | | | | | | | | | | linoleoyl |
| | | | | | | | | | | GPC |
| 63 | MM_F1_6 | F1 | logistic | 0.5 | 0.58 | 0.91 | 0.76 | 0.82 | 0.85 | insulin, |
| | | | regression | | | | | | | AHB, |
| | | | | | | | | | | linoleoyl |
| | | | | | | | | | | GPC, |
| | | | | | | | | | | BMI |
| 64 | MM_F1_7 | F1 | logistic | 0.5 | 0.61 | 0.91 | 0.76 | 0.83 | 0.86 | insulin, |
| | | | regression | | | | | | | AHB, |
| | | | | | | | | | | linoleoyl |
| | | | | | | | | | | GPC, |
| | | | | | | | | | | decanoyl |
| | | | | | | | | | | carnitine |
| 65 | RF_F1_1 | F1 | random | | 0.72 | 0.75 | 0.61 | 0.84 | 0.79 | BMI, 2- |
| | | | forest | | | | | | | hydroxy |
| | | | | | | | | | | butyrate, |
| | | | | | | | | | | Linoleoyl- |
| | | | | | | | | | | LPC |
| 66 | RF_F1_2 | F1 | random | | 0.73 | 0.74 | 0.60 | 0.84 | 0.80 | BMI, 2- |
| | | | forest | | | | | | | hydroxy |
| | | | | | | | | | | butyrate, |
| | | | | | | | | | | Linoleoyl- |
| | | | | | | | | | | LPC, |
| | | | | | | | | | | glycine |
| 67 | RF_F1_3 | F1 | random | | 0.71 | 0.75 | 0.59 | 0.84 | 0.82 | Insulin, |
| | | | forest | | | | | | | 2- |
| | | | | | | | | | | hydroxy |
| | | | | | | | | | | butyrate, |
| | | | | | | | | | | Linoleoyl- |
| | | | | | | | | | | LPC |
| 68 | RF_F1_4 | F1 | random | | 0.71 | 0.77 | 0.61 | 0.84 | 0.82 | Insulin, |
| | | | forest | | | | | | | 2- |
| | | | | | | | | | | hydroxy |
| | | | | | | | | | | butyrate, |
| | | | | | | | | | | Linoleoyl- |
| | | | | | | | | | | LPC, |
| | | | | | | | | | | BMI |
| 69 | RF_F1_5 | F1 | random | | 0.73 | 0.78 | 0.62 | 0.85 | 0.82 | Insulin, |
| | | | forest | | | | | | | 2- |
| | | | | | | | | | | hydroxy |
| | | | | | | | | | | butyrate, |
| | | | | | | | | | | Linoleoyl- |
| | | | | | | | | | | LPC, |
| | | | | | | | | | | BMI, |
| | | | | | | | | | | glycine |
| 70 | CH_F2_1a | F2 | logistic | 0.3 | 0.79 | 0.81 | 0.63 | 0.91 | 0.88 | |
| | | | regression | | | | | | | |
| 71 | CH_F2_1b | F2 | logistic | 0.3 | 0.78 | 0.85 | 0.66 | 0.91 | 0.90 | |
| | | | regression | | | | | | | |
| 72 | MM_F2_1 | F2 | logistic | 0.3 | 0.70 | 0.84 | 0.61 | 0.88 | 0.86 | AHB, |
| | | | regression | | | | | | | insulin |
| 73 | MM_F2_2 | F2 | logistic | 0.3 | 0.75 | 0.82 | 0.60 | 0.90 | 0.87 | AHB, |
| | | | regression | | | | | | | insulin, |
| | | | | | | | | | | linoleoyl |
| | | | | | | | | | | GPC |
| 74 | MM_F2_3 | F2 | logistic | 0.3 | 0.74 | 0.84 | 0.63 | 0.90 | 0.87 | AHB, |
| | | | regression | | | | | | | insulin, |
| | | | | | | | | | | BMI |
| 75 | MM_F2_4 | F2 | logistic | 0.3 | 0.73 | 0.81 | 0.61 | 0.89 | 0.84 | AHB, |
| | | | regression | | | | | | | BMI |
| 76 | MM_F2_5 | F2 | logistic | 0.3 | 0.73 | 0.78 | 0.57 | 0.88 | 0.85 | AHB, |
| | | | regression | | | | | | | BMI, |
| | | | | | | | | | | linoleoyl |
| | | | | | | | | | | GPC |
| 77 | CH_F2_1a | F2 | logistic | 0.5 | 0.61 | 0.95 | 0.82 | 0.86 | 0.88 | |
| | | | regression | | | | | | | |
| 78 | CH_F2_1b | F2 | logistic | 0.5 | 0.63 | 0.94 | 0.79 | 0.87 | 0.90 | |
| | | | regression | | | | | | | |
| 79 | MM_F2_1 | F2 | logistic | 0.5 | 0.55 | 0.93 | 0.75 | 0.85 | 0.86 | AHB, |
| | | | regression | | | | | | | insulin |
| 80 | MM_F2_2 | F2 | logistic | 0.5 | 0.54 | 0.94 | 0.76 | 0.85 | 0.87 | AHB, |
| | | | regression | | | | | | | insulin, |
| | | | | | | | | | | linoleoyl |
| | | | | | | | | | | GPC |
| 81 | MM_F2_3 | F2 | logistic | 0.5 | 0.52 | 0.93 | 0.74 | 0.84 | 0.87 | AHB, |
| | | | regression | | | | | | | insulin, |
| | | | | | | | | | | BMI |
| 82 | MM_F2_4 | F2 | logistic | 0.5 | 0.50 | 0.91 | 0.70 | 0.82 | 0.84 | AHB, |
| | | | regression | | | | | | | BMI |
| 83 | MM_F2_5 | F2 | logistic | 0.5 | 0.48 | 0.91 | 0.68 | 0.82 | 0.85 | AHB, |
| | | | regression | | | | | | | BMI, |
| | | | | | | | | | | linoleoyl |
| | | | | | | | | | | GPC |
| 84 | RF_F2_1 | F2 | random | | 0.77 | 0.74 | 0.53 | 0.89 | 0.82 | BMI, 2- |
| | | | forest | | | | | | | hydroxy |
| | | | | | | | | | | butyrate, |
| | | | | | | | | | | Linoleoyl- |
| | | | | | | | | | | LPC |
| 85 | RF_F2_2 | F2 | random | | 0.74 | 0.75 | 0.53 | 0.88 | 0.83 | BMI, 2- |
| | | | forest | | | | | | | hydroxy |
| | | | | | | | | | | butyrate, |
| | | | | | | | | | | Linoleoyl- |
| | | | | | | | | | | LPC, |
| | | | | | | | | | | glycine |
| 86 | RF_F2_3 | F2 | random | | 0.76 | 0.81 | 0.59 | 0.91 | 0.85 | Insulin, |
| | | | forest | | | | | | | 2- |
| | | | | | | | | | | hydroxy |
| | | | | | | | | | | butyrate, |
| | | | | | | | | | | Linoleoyl- |
| | | | | | | | | | | LPC |
| 87 | RF_F2_4 | F2 | random | | 0.79 | 0.77 | 0.55 | 0.91 | 0.85 | Insulin, |
| | | | forest | | | | | | | 2- |
| | | | | | | | | | | hydroxy |
| | | | | | | | | | | butyrate, |
| | | | | | | | | | | Linoleoyl- |
| | | | | | | | | | | LPC, |
| | | | | | | | | | | BMI |
| 88 | RF_F2_5 | F2 | random | | 0.79 | 0.77 | 0.55 | 0.91 | 0.86 | Insulin, |
| | | | forest | | | | | | | 2- |
| | | | | | | | | | | hydroxy |
| | | | | | | | | | | butyrate, |
| | | | | | | | | | | Linoleoyl- |
| | | | | | | | | | | LPC, |
| | | | | | | | | | | BMI, |
| | | | | | | | | | | glycine |
| 89 | CH_F3_1a | F3 | logistic | 0.3 | 0.72 | 0.85 | 0.62 | 0.90 | 0.87 | |
| | | | regression | | | | | | | |
| 90 | CH_F3_1b | F3 | logistic | 0.3 | 0.74 | 0.89 | 0.68 | 0.92 | 0.89 | |
| | | | regression | | | | | | | |
| 91 | MM_F3_1 | F3 | logistic | 0.3 | 0.68 | 0.83 | 0.57 | 0.89 | 0.84 | AHB, |
| | | | regression | | | | | | | BMI |
| 92 | MM_F3_2 | F3 | logistic | 0.3 | 0.71 | 0.82 | 0.57 | 0.89 | 0.85 | AHB, |
| | | | regression | | | | | | | linoleoyl |
| | | | | | | | | | | GPC, |
| | | | | | | | | | | BMI |
| 93 | MM_F3_3 | F3 | logistic | 0.3 | 0.73 | 0.87 | 0.64 | 0.91 | 0.86 | AHB, |
| | | | regression | | | | | | | insulin |
| 94 | MM_F3_4 | F3 | logistic | 0.3 | 0.74 | 0.86 | 0.63 | 0.91 | 0.87 | AHB, |
| | | | regression | | | | | | | insulin, |
| | | | | | | | | | | BMI |
| 95 | MM_F3_5 | F3 | logistic | 0.3 | 0.75 | 0.87 | 0.64 | 0.92 | 0.88 | AHB, |
| | | | regression | | | | | | | insulin, |
| | | | | | | | | | | linoleoyl |
| | | | | | | | | | | GPC |
| 96 | CH_F3_1a | F3 | logistic | 0.5 | 0.50 | 0.95 | 0.76 | 0.85 | 0.87 | |
| | | | regression | | | | | | | |
| 97 | CH_F3_1b | F3 | logistic | 0.5 | 0.56 | 0.94 | 0.74 | 0.87 | 0.89 | |
| | | | regression | | | | | | | |
| 98 | MM_F3_1 | F3 | logistic | 0.5 | 0.44 | 0.95 | 0.73 | 0.83 | 0.84 | AHB, |
| | | | regression | | | | | | | BMI |
| 99 | MM_F3_2 | F3 | logistic | 0.5 | 0.45 | 0.94 | 0.70 | 0.84 | 0.85 | AHB, |
| | | | regression | | | | | | | linoleoyl |
| | | | | | | | | | | GPC, |
| | | | | | | | | | | BMI |
| 100 | MM_F3_3 | F3 | logistic | 0.5 | 0.49 | 0.94 | 0.71 | 0.85 | 0.86 | AHB, |
| | | | regression | | | | | | | insulin |
| 101 | MM_F3_4 | F3 | logistic | 0.5 | 0.52 | 0.93 | 0.71 | 0.86 | 0.87 | AHB, |
| | | | regression | | | | | | | insulin, |
| | | | | | | | | | | BMI |
| 102 | MM_F3_5 | F3 | logistic | 0.5 | 0.52 | 0.94 | 0.73 | 0.86 | 0.88 | AHB, |
| | | | regression | | | | | | | insulin, |
| | | | | | | | | | | linoleoyl |
| | | | | | | | | | | GPC |
| 103 | RF_F3_1 | F3 | random | | 0.74 | 0.73 | 0.46 | 0.90 | 0.82 | BMI, 2- |
| | | | forest | | | | | | | hydroxy |
| | | | | | | | | | | butyrate, |
| | | | | | | | | | | Linoleoyl- |
| | | | | | | | | | | LPC |
| 104 | RF_F3_2 | F3 | random | | 0.75 | 0.73 | 0.47 | 0.90 | 0.83 | BMI, 2- |
| | | | forest | | | | | | | hydroxy |
| | | | | | | | | | | butyrate, |
| | | | | | | | | | | Linoleoyl- |
| | | | | | | | | | | LPC, |
| | | | | | | | | | | glycine |
| 105 | RF_F3_3 | F3 | random | | 0.80 | 0.80 | 0.54 | 0.93 | 0.86 | Insulin, |
| | | | forest | | | | | | | 2- |
| | | | | | | | | | | hydroxy |
| | | | | | | | | | | butyrate, |
| | | | | | | | | | | Linoleoyl- |
| | | | | | | | | | | LPC |
| 106 | RF_F3_4 | F3 | random | | 0.78 | 0.78 | 0.51 | 0.92 | 0.86 | Insulin, |
| | | | forest | | | | | | | 2- |
| | | | | | | | | | | hydroxy |
| | | | | | | | | | | butyrate, |
| | | | | | | | | | | Linoleoyl, |
| | | | | | | | | | | LPC, |
| | | | | | | | | | | BMI |
| 107 | RF_F3_5 | F3 | random | | 0.80 | 0.79 | 0.53 | 0.93 | 0.87 | Insulin, |
| | | | forest | | | | | | | 2- |
| | | | | | | | | | | hydroxy |
| | | | | | | | | | | butyrate, |
| | | | | | | | | | | Linoleoyl- |
| | | | | | | | | | | LPC, |
| | | | | | | | | | | BMI, |
| | | | | | | | | | | glycine |

¹ Response for the Logistic Regression models in Table 18 is defined as follows:
F1: IR defined as M_ffm <= 45
F2: IR defined as M_ffm <= 39
F3: IR defined as M_ffm <= 37
G1: IR defined as M_wbm <= 5.6
G2: IR defined as M_wbm <= 5
G3: IR defined as M_wbm <= 4.5
² “octano_decano_mean” is the average of decanoyl_carnitine and octanoyl_carnitine
³Risk Score Models only applied to G1
RS1
BMI >= 26.55
2-hydroxybutyrate >= 5.08021
Linoleoyl-LPGC < 15.60359
insulin >= 35.925
One point is assigned to each condition satisfied (thus, 0-4 are the possible scores)

### Example 11

### Correlation Analysis of IR Biomarkers

Many biomarker compounds were correlated as shown in Table 19 and Table 20. Table 19 contains a matrix showing the pair-wise correlation analysis of biomarkers based upon quantitative data obtained from the targeted assays. Table 20 contains pair-wise correlations of the screening data for compounds for which targeted assays have not yet been developed. In addition, the correlation between selected clinical parameters of IR and biomarkers are presented in Table 20. Correlated compounds are often mutually exclusive in regression models and thus can be used (i.e. substituted for a correlated compound) in different models that had similar prediction powers as those shown in Table 17 (models table) above.

TABLE 19
Biomarker Correlation Matrix

| | 1 | 2 | 3 | 4 | 5 | 6 | 7 |
|---|---|---|---|---|---|---|---|
| | 2-hydroxybutyrate | 3-hydroxy-butyrate | 3-methyl-2-oxo-butyric_acid | arginine | betaine | Creatine | decanoyl_carnitine |
| 1 | 1.00 | 0.46 | 0.35 | −0.11 | −0.05 | 0.30 | −0.01 |
| 2 | 0.46 | 1.00 | 0.04 | −0.02 | −0.03 | 0.04 | 0.19 |
| 3 | 0.35 | 0.04 | 1.00 | −0.11 | 0.06 | 0.05 | 0.00 |
| 4 | −0.11 | −0.02 | −0.11 | 1.00 | 0.08 | 0.07 | −0.01 |
| 5 | −0.05 | −0.03 | 0.06 | 0.08 | 1.00 | −0.32 | 0.08 |
| 6 | 0.30 | 0.04 | 0.05 | 0.07 | −0.32 | 1.00 | −0.23 |
| 7 | −0.01 | 0.19 | 0.00 | −0.01 | 0.08 | −0.23 | 1.00 |
| 8 | 0.38 | 0.39 | 0.03 | 0.01 | −0.03 | 0.07 | 0.31 |
| 9 | −0.04 | 0.05 | −0.35 | 0.08 | 0.17 | −0.13 | 0.16 |
| 10 | 0.02 | −0.02 | −0.38 | 0.06 | 0.06 | −0.03 | 0.02 |
| 11 | −0.05 | 0.04 | −0.36 | 0.10 | 0.11 | −0.09 | 0.13 |
| 12 | −0.33 | −0.10 | −0.20 | 0.18 | 0.05 | −0.02 | 0.03 |
| 13 | 0.29 | 0.43 | −0.04 | 0.04 | −0.13 | 0.13 | 0.27 |
| 14 | 0.24 | 0.45 | 0.00 | −0.01 | −0.01 | 0.09 | 0.32 |
| 15 | 0.19 | 0.38 | 0.00 | 0.12 | −0.13 | 0.10 | 0.21 |
| 16 | −0.34 | −0.19 | −0.05 | 0.06 | 0.29 | −0.35 | 0.10 |
| 17 | 0.42 | 0.53 | 0.09 | 0.03 | −0.11 | 0.14 | 0.26 |
| 18 | 0.03 | 0.19 | 0.05 | −0.03 | 0.09 | −0.20 | 0.98 |
| 19 | 0.39 | 0.60 | 0.01 | 0.05 | −0.09 | 0.16 | 0.28 |
| 20 | −0.23 | −0.16 | −0.10 | 0.03 | 0.16 | −0.25 | 0.05 |
| 21 | 0.40 | 0.53 | 0.05 | 0.07 | −0.14 | 0.14 | 0.31 |
| 22 | 0.23 | 0.41 | −0.07 | 0.11 | −0.16 | 0.13 | 0.24 |
| 23 | −0.15 | −0.17 | −0.18 | −0.04 | 0.08 | −0.12 | 0.01 |
| 24 | −0.03 | 0.18 | −0.03 | 0.00 | 0.13 | 0.03 | 0.03 |
| 25 | 0.45 | 0.57 | 0.15 | 0.07 | −0.12 | 0.11 | 0.25 |
| 26 | −0.22 | −0.20 | −0.18 | −0.02 | 0.22 | −0.17 | 0.00 |
| 27 | −0.13 | −0.12 | 0.01 | 0.17 | 0.05 | 0.08 | −0.09 |
| 28 | −0.11 | −0.30 | 0.22 | 0.08 | 0.17 | −0.15 | 0.08 |

| | 8 | 9 | 10 | 11 | 12 | 13 |
|---|---|---|---|---|---|---|
| | Docosatetraenoic_acid | gamma-glutamyl-leucine | glutamic_acid | glutamyl-valine | glycine | Heptadeoenoic_acid |
| 1 | 0.38 | −0.04 | 0.02 | −0.05 | −0.33 | 0.29 |
| 2 | 0.39 | 0.05 | −0.02 | 0.04 | −0.10 | 0.43 |
| 3 | 0.03 | −0.35 | −0.38 | −0.36 | −0.20 | −0.04 |
| 4 | 0.01 | 0.08 | 0.06 | 0.10 | 0.18 | 0.04 |
| 5 | −0.03 | 0.17 | 0.06 | 0.11 | 0.05 | −0.13 |
| 6 | 0.07 | −0.13 | −0.03 | −0.09 | −0.02 | 0.13 |
| 7 | 0.31 | 0.16 | 0.02 | 0.13 | 0.03 | 0.27 |
| 8 | 1.00 | 0.14 | 0.12 | 0.12 | −0.15 | 0.73 |
| 9 | 0.14 | 1.00 | 0.83 | 0.98 | 0.00 | 0.03 |
| 10 | 0.12 | 0.83 | 1.00 | 0.81 | −0.10 | 0.03 |
| 11 | 0.12 | 0.98 | 0.81 | 1.00 | −0.01 | 0.03 |
| 12 | −0.15 | 0.00 | −0.10 | −0.01 | 1.00 | −0.06 |
| 13 | 0.73 | 0.03 | 0.03 | 0.03 | −0.06 | 1.00 |
| 14 | 0.72 | 0.05 | 0.01 | 0.02 | −0.05 | 0.67 |
| 15 | 0.42 | −0.02 | −0.07 | −0.03 | −0.05 | 0.63 |
| 16 | −0.23 | 0.12 | 0.03 | 0.08 | 0.26 | −0.22 |
| 17 | 0.67 | 0.07 | 0.05 | 0.07 | −0.11 | 0.81 |
| 18 | 0.32 | 0.11 | −0.01 | 0.09 | 0.02 | 0.26 |
| 19 | 0.68 | −0.01 | −0.01 | −0.03 | −0.09 | 0.81 |
| 20 | −0.25 | 0.19 | 0.16 | 0.16 | 0.20 | −0.22 |
| 21 | 0.76 | 0.05 | 0.05 | 0.04 | −0.15 | 0.86 |
| 22 | 0.63 | −0.03 | 0.00 | −0.02 | −0.05 | 0.86 |
| 23 | −0.12 | 0.38 | 0.38 | 0.35 | 0.07 | −0.22 |
| 24 | −0.01 | 0.01 | −0.04 | −0.02 | 0.54 | 0.06 |
| 25 | 0.61 | 0.03 | 0.03 | 0.03 | −0.14 | 0.65 |
| 26 | −0.10 | 0.32 | 0.30 | 0.29 | 0.15 | −0.20 |
| 27 | −0.07 | 0.04 | 0.00 | 0.03 | 0.29 | −0.07 |
| 28 | −0.15 | 0.13 | 0.13 | 0.08 | 0.06 | −0.25 |

| | 14 | 15 | 16 | 17 | 18 | 19 | 20 | 21 |
|---|---|---|---|---|---|---|---|---|
| | linoleic_acid | linolenic_acid | Linoleoyl-LPC | margaric_acid | octanoyl_carnitine | oleic_acid | oleoyl-LPC | palmitate |
| 1 | 0.24 | 0.19 | −0.34 | 0.42 | 0.03 | 0.39 | −0.23 | 0.40 |
| 2 | 0.45 | 0.38 | −0.19 | 0.53 | 0.19 | 0.60 | −0.16 | 0.53 |
| 3 | 0.00 | 0.00 | −0.05 | 0.09 | 0.05 | 0.01 | −0.10 | 0.05 |
| 4 | −0.01 | 0.12 | 0.06 | 0.03 | −0.03 | 0.05 | 0.03 | 0.07 |
| 5 | −0.01 | −0.13 | 0.29 | −0.11 | 0.09 | −0.09 | 0.16 | −0.14 |
| 6 | 0.09 | 0.10 | −0.35 | 0.14 | −0.20 | 0.16 | −0.25 | 0.14 |
| 7 | 0.32 | 0.21 | 0.10 | 0.26 | 0.98 | 0.28 | 0.05 | 0.31 |
| 8 | 0.72 | 0.42 | −0.23 | 0.67 | 0.32 | 0.68 | −0.25 | 0.76 |
| 9 | 0.05 | −0.02 | 0.12 | 0.07 | 0.11 | −0.01 | 0.19 | 0.05 |
| 10 | 0.01 | −0.07 | 0.03 | 0.05 | −0.01 | −0.01 | 0.16 | 0.05 |
| 11 | 0.02 | −0.03 | 0.08 | 0.07 | 0.09 | −0.03 | 0.16 | 0.04 |
| 12 | −0.05 | −0.05 | 0.26 | −0.11 | 0.02 | −0.09 | 0.20 | −0.15 |
| 13 | 0.67 | 0.63 | −0.22 | 0.81 | 0.26 | 0.81 | −0.22 | 0.86 |
| 14 | 1.00 | 0.55 | −0.13 | 0.73 | 0.35 | 0.75 | −0.25 | 0.76 |
| 15 | 0.55 | 1.00 | −0.14 | 0.56 | 0.18 | 0.65 | −0.13 | 0.70 |
| 16 | −0.13 | −0.14 | 1.00 | −0.21 | 0.10 | −0.32 | 0.68 | −0.28 |
| 17 | 0.73 | 0.56 | −0.21 | 1.00 | 0.26 | 0.81 | −0.18 | 0.89 |
| 18 | 0.35 | 0.18 | 0.10 | 0.26 | 1.00 | 0.27 | 0.02 | 0.30 |
| 19 | 0.75 | 0.65 | −0.32 | 0.81 | 0.27 | 1.00 | −0.16 | 0.93 |
| 20 | −0.25 | −0.13 | 0.68 | −0.18 | 0.02 | −0.16 | 1.00 | −0.20 |
| 21 | 0.76 | 0.70 | −0.28 | 0.89 | 0.30 | 0.93 | −0.20 | 1.00 |
| 22 | 0.61 | 0.66 | −0.29 | 0.65 | 0.23 | 0.83 | −0.14 | 0.85 |
| 23 | −0.10 | −0.15 | 0.41 | −0.13 | −0.01 | −0.19 | 0.71 | −0.15 |
| 24 | 0.11 | −0.04 | 0.18 | 0.08 | 0.04 | 0.16 | 0.18 | 0.04 |
| 25 | 0.64 | 0.56 | −0.19 | 0.87 | 0.24 | 0.76 | −0.14 | 0.84 |
| 26 | −0.01 | −0.15 | 0.49 | −0.11 | 0.00 | −0.19 | 0.61 | −0.18 |
| 27 | −0.05 | −0.14 | 0.18 | −0.08 | −0.08 | −0.04 | 0.18 | −0.09 |
| 28 | −0.20 | −0.24 | 0.31 | −0.20 | 0.06 | −0.30 | 0.27 | −0.24 |

| | 22 | 23 | 24 | 25 | 26 | 27 | 28 |
|---|---|---|---|---|---|---|---|
| | palmitoleic_acid | palmitoyl-LPC | serine | stearate | stearoyl-LPC | threonine | tryptophan |
| 1 | 0.23 | −0.15 | −0.03 | 0.45 | −0.22 | −0.13 | −0.11 |
| 2 | 0.41 | −0.17 | 0.18 | 0.57 | −0.20 | −0.12 | −0.30 |
| 3 | −0.07 | −0.18 | −0.03 | 0.15 | −0.18 | 0.01 | 0.22 |
| 4 | 0.11 | −0.04 | 0.00 | 0.07 | −0.02 | 0.17 | 0.08 |
| 5 | −0.16 | 0.08 | 0.13 | −0.12 | 0.22 | 0.05 | 0.17 |
| 6 | 0.13 | −0.12 | 0.03 | 0.11 | −0.17 | 0.08 | −0.15 |
| 7 | 0.24 | 0.01 | 0.03 | 0.25 | 0.00 | −0.09 | 0.08 |
| 8 | 0.63 | −0.12 | −0.01 | 0.61 | −0.10 | −0.07 | −0.15 |
| 9 | −0.03 | 0.38 | 0.01 | 0.03 | 0.32 | 0.04 | 0.13 |
| 10 | 0.00 | 0.38 | −0.04 | 0.03 | 0.30 | 0.00 | 0.13 |
| 11 | −0.02 | 0.35 | −0.02 | 0.03 | 0.29 | 0.03 | 0.08 |
| 12 | −0.05 | 0.07 | 0.54 | −0.14 | 0.15 | 0.29 | 0.06 |
| 13 | 0.86 | −0.22 | 0.06 | 0.65 | −0.20 | −0.07 | −0.25 |
| 14 | 0.61 | −0.10 | 0.11 | 0.64 | −0.01 | −0.05 | −0.20 |
| 15 | 0.66 | −0.15 | −0.04 | 0.56 | −0.15 | −0.14 | −0.24 |
| 16 | −0.29 | 0.41 | 0.18 | −0.19 | 0.49 | 0.18 | 0.31 |
| 17 | 0.65 | −0.13 | 0.08 | 0.87 | −0.11 | −0.08 | −0.20 |
| 18 | 0.23 | −0.01 | 0.04 | 0.24 | 0.00 | −0.08 | 0.06 |
| 19 | 0.83 | −0.19 | 0.16 | 0.76 | −0.19 | −0.04 | −0.30 |
| 20 | −0.14 | 0.71 | 0.18 | −0.14 | 0.61 | 0.18 | 0.27 |
| 21 | 0.85 | −0.15 | 0.04 | 0.84 | −0.18 | −0.09 | −0.24 |
| 22 | 1.00 | −0.14 | 0.03 | 0.57 | −0.21 | −0.08 | −0.27 |
| 23 | −0.14 | 1.00 | 0.06 | −0.13 | 0.80 | 0.09 | 0.26 |
| 24 | 0.03 | 0.06 | 1.00 | 0.04 | 0.13 | 0.46 | −0.02 |
| 25 | 0.57 | −0.13 | 0.04 | 1.00 | −0.10 | −0.10 | −0.19 |
| 26 | −0.21 | 0.80 | 0.13 | −0.10 | 1.00 | 0.09 | 0.25 |
| 27 | −0.08 | 0.09 | 0.46 | −0.10 | 0.09 | 1.00 | 0.12 |
| 28 | −0.27 | 0.26 | −0.02 | −0.19 | 0.25 | 0.12 | 1.00 |

TABLE 20
Correlated Biomarkers and Clinical Parameters

| Pairwise Correlation | Correlation |
|---|---|
| 1,5-anhydroglucitol-1,5 (AG) *alpha-ketobutyrate | −0.5046 |
| 2-hydroxybutyrate (AHB)*1,5-anhydroglucitol-1,5 (AG) | −0.5413 |
| 2-hydroxybutyrate (AHB)*alpha-ketobutyrate | 0.8857 |
| galactonic acid*alpha-ketobutyrate | 0.6051 |
| gluconate*alpha-ketobutyrate | 0.516 |
| margarate (17:0)*alpha-ketobutyrate | 0.5374 |
| palmitate (16:0)*alpha-ketobutyrate | 0.5431 |
| stearate (18:0)*alpha-ketobutyrate | 0.5859 |
| glutamate*1,5-anhydroglucitol-1,5 (AG) | −0.6945 |
| glutamate*alpha-ketobutyrate | 0.6742 |
| HDL_Cholesterol*Adiponectin | 0.511148 |
| Fat_Mass*BMI | 0.843078 |
| Weight*BMI | 0.804681 |
| Waist*BMI | 0.800452 |
| Hip*BMI | 0.705318 |
| Fat_Mass_pcnt*BMI | 0.602829 |
| BMI*HOMA | 0.590842 |
| BMI*Fasting_Insulin | 0.589749 |
| BMI*QUICKI | −0.580267 |
| RD*BMI | −0.551166 |
| BMI*Fasting_C_Peptide | 0.542661 |
| Fasting_C_Peptide*HOMA | 0.829625 |
| Fasting_Insulin*Fasting_C_Peptide | 0.828392 |
| Fasting_C_Peptide*QUICKI | −0.768811 |
| Fasting_Proinsulin*Fasting_C_Peptide | 0.570761 |
| Fat_Mass*Fasting_C_Peptide | 0.519632 |
| RD*Fasting_C_Peptide | −0.506727 |
| Waist*Fasting_C_Peptide | 0.501492 |
| Fasting_Insulin*HOMA | 0.979376 |
| Fasting_Insulin*QUICKI | −0.880137 |
| Fasting_Insulin*Fasting_Proinsulin | 0.509757 |
| Fat_Mass*Fasting_Insulin | 0.576818 |
| Waist*Fasting_Insulin | 0.502325 |
| Fasting_Proinsulin*HOMA | 0.52513 |
| Fasting_FFA*palmitate (16:0) | 0.552703 |
| Fasting_FFA*oleate (18:1(n-9)) | 0.519978 |
| Fasting_FFA*linoleate (18:2(n-6)) | 0.504094 |
| Fasting_FFA*Heptadecenate | 0.503364 |
| Fasting_FFA*Heptadecenate | 0.503364 |

### Example 12

### Classification of IGT

Biomarkers 1-24 of Table 4 were used to classify the subjects described in Table 21 according to glucose tolerance. Using the oral glucose tolerance test (OGTT), where IGT is defined as 2-hr OGTT>=140, the subjects were classified as having normal glucose tolerance (NGT) or impaired glucose tolerance (IGT). Using the targeted analytical methods described in Example 8, the levels of biomarkers 1-24 in Table 4 were measured in plasma samples collected from the fasting subjects and the results were subjected to statistical analysis. Statistical significance testing of the biomarkers was performed using the t-test and the subjects were classified as NGT or IGT using Random Forest analysis.

TABLE 21
Cohort Description of NGT and IGT Subjects

| | Mean Age | Mean BMI | % Male | % Female | N |
|---|---|---|---|---|---|
| NGT | 43.6 | 25.29 | 45.26 | 54.74 | 317 |
| IGT | 46.07 | 27.59 | 40.17 | 59.83 | 82 |

The results of the Random Forest analysis show that measuring the biomarkers in samples collected from NGT subjects and IGT subjects can classify the subjects as NGT or IGT with ˜63% accuracy without including BMI and ˜64% if BMI is included in the analysis. The results are shown in the confusion matrix in Table 22. The analysis also orders the biomarkers from most important to least important to distinguish the subjects as NGT or IGT. The order from most important to least important is: 2-hydroxybutyrate, creatine, palmitate, glutamate, stearate, adrenate, oleic acid, decanoyl carnitine, linoleoyl-LPC, octanoyl carnitine, 3-hyroxy-butyrate, margaric acid, glycine, oleoyl-LPC, palmitoleic acid, linoleic acid, 3-methyl-2-oxo-butyric acid, palmitoyl-LPC, tryptophan, serine, arginine, threonine, linolenic acid, betaine. If BMI is included, the order from most important to least important is: 2-hydroxybutyrate, creatine, BMI, palmitate, stearate, glutamate, oleic acid, adernate, decanoyl carnitine, linoleoyl-LPC, margaric acid, octanoyl carnitine, palmitoleic acid, 3-hydroxybutyrate, glycine, oleoyl-LPC, linoleic acid, 3-methyl-2-oxo-butyric acid, palmitoyl-LPC, tryptophan, linolenic acid, threonine, serine, arginine, betaine.

TABLE 22
Confusion Matrix to Classify Subjects as NGT or IGT without (Top)
or with (Bottom) BMI as a variable.

| | IGT | NGT | Error |
|---|---|---|---|
| IGT | 59 | 23 | 0.2805 |
| NGT | 86 | 231 | 0.2713 |

OOB Estimate of Error 27.32%

| | | | |
|---|---|---|---|
| IGT | 58 | 24 | 0.2927 |
| NGT | 83 | 234 | 0.2618 |

OOB Estimate of Error 26.82%

The results were also analyzed using the t-test to determine the most significant biomarkers for classifying subjects as NGT or IGT. These results are presented in Table 23.

TABLE 23
T-test results of biomarkers for classification of NGT from IGT
subjects.

| | | | IGT | NGT | |
|---|---|---|---|---|---|
| Biomarker | p-value | q-value | (Mean) | (Mean) | RATIO |
| 2-hydroxybutyrate | 1.05E−12 | 3.50E−12 | 5.92 | 4.23 | 1.4 |
| creatine | 8.12E−10 | 1.35E−09 | 5.83 | 3.93 | 1.48 |
| BMI | 1.33E−08 | 1.48E−08 | 28 | 24.87 | 1.13 |
| linoleoyl-LPC | 8.43E−08 | 7.03E−08 | 14.08 | 17.41 | 0.81 |
| oleic_acid | 1.31E−07 | 8.19E−08 | 103.36 | 81.42 | 1.27 |
| adrenate | 1.47E−07 | 8.19E−08 | 0.22 | 0.18 | 1.24 |
| palmitate | 3.08E−07 | 1.47E−07 | 39.34 | 31.83 | 1.24 |
| stearic_acid | 1.26E−06 | 5.25E−07 | 14.54 | 12.11 | 1.2 |
| margaric_acid | 4.13E−06 | 1.53E−06 | 0.45 | 0.37 | 1.2 |
| oleoyl-LPC | 1.09E−05 | 3.65E−06 | 9.54 | 11.31 | 0.84 |
| glycine | 9.24E−05 | 2.80E−05 | 23.62 | 26.74 | 0.88 |
| linoleic_acid | 0.0001 | 3.45E−05 | 17.41 | 14.75 | 1.18 |
| 3-hydroxy-butyrate | 0.0009 | 0.0002 | 8.43 | 5.92 | 1.42 |
| palmitoyl-LPC | 0.0023 | 0.0006 | 17.5 | 19.41 | 0.9 |
| linolenic_acid | 0.0031 | 0.0007 | 3.72 | 3.11 | 1.2 |
| glutamate | 0.0083 | 0.0016 | 16.83 | 15.16 | 1.11 |
| palmitoleic_acid | 0.0084 | 0.0016 | 7.56 | 6.34 | 1.19 |
| tryptophan | 0.0205 | 0.0036 | 5.23 | 5.48 | 0.95 |
| 3-methyl-2-oxo- | 0.0206 | 0.0036 | 2.37 | 2.19 | 1.08 |
| butyric_acid | | | | | |
| decanoyl_carnitine | 0.096 | 0.016 | 0.05 | 0.06 | 0.82 |
| serine | 0.223 | 0.0347 | 10.58 | 10.9 | 0.97 |
| arginine | 0.2288 | 0.0347 | 17.03 | 16.48 | 1.03 |
| betaine | 0.3386 | 0.0491 | 4.24 | 4.31 | 0.98 |
| octanoyl_carnitine | 0.3774 | 0.0524 | 0.03 | 0.03 | 0.88 |
| threonine | 0.864 | 0.1153 | 15.35 | 15.3 | 1 |

### Example 13

### Prediction of Progression to IR-Associated Disorders

Biomarkers 1-24 listed in Table 4 were used to identify the subjects described in Table 24 that will progress from normoglycemia to dysglycemia. For example, subjects may become increasingly dysglycemic and eventually progress from NGT to IGT and/or Type II Diabetes. Using the oral glucose tolerance test, where IGT is defined as 2-hr OGTT>=140, the subjects were classified as having normal glucose tolerance (NGT) or impaired glucose tolerance (IGT) at baseline and again after 3 years. Subjects that had OGTT<140 at baseline and OGTT>=140 at 3 years and the difference in the OGTT measurements is at least 10 units were defined as “progressors” and subjects that had OGTT<140 at both time points were defined as “non-progressors” (stable NGT). Using the targeted analytical methods described in Example 8, the levels of the biomarkers 1-25 in Table 4 were measured in plasma samples collected from the fasting subjects at baseline and the results were subjected to statistical analysis. Statistical significance testing of the biomarkers was performed using the t-test and the subjects were classified as “progressors” or “non-progressors” using Random Forest analysis.

TABLE 24
Cohort Description of Non-Progressors vs. Progressors

| Condition | Non-progressors | Progressors |
|---|---|---|
| Dysglycemia | 842 | 82 |
| Dyslipidemia | 796 | 69 |

Likewise, the subjects that progressed to the IR-associated disorder of dyslipidemia were identified using the 3 year outcome data. The ability of the biomarkers to predict which subjects will progress to each condition was determined based upon the levels of the biomarkers measured in the baseline samples. The results obtained from the biomarker assays were analyzed statistically using t-tests and Random Forest analysis as described above. The 3 year outcome data was measured using the parameters set forth below in Table 25.

TABLE 25
IR-associated Disease Outcomes and Associated Clinical Parameters

| DISEASE | VARIABLE | CLINICAL RISK | DISEASE |
|---|---|---|---|
| OUTCOME | MEASURED | CUT-OFF | CUT-OFF |
| Impaired Glucose | OGTT | >140-199 mg/dL | ≧200 mg/dL |
| Tolerance/Type II | | (IGT) | (T2D) |
| Diabetes | | | |
| Dyslipidemia | HDL | <40 mg/dL | |

According to Guidelines from National Cholesterol Education Program Adult Treatment Panel III, American Heart Assoc, National Heart Lung Blood Institute of NIH

The results of the Random Forest analysis shows that measuring the biomarkers in baseline samples can predict the subjects that will progress to dysglycemia at 3 years with ˜64% accuracy without including BMI and ˜65% if BMI is included in the analysis. The results are shown in the confusion matrix in Table 26. The analysis also orders the biomarkers from most important to least important to distinguish the subjects that will progress to dysglycemia from those who will not progress (i.e., remain normoglycemic). The order from most important to least important is: linoleoyl-LPC, 3-hydroxy-butyrate, threonine, creatine, betaine, palmitoyl-LPC, oleoyl-LPC, glycine, 2-hydroxybutyrate, glutamic acid, oleic acid, decanoyl carnitine, octanoyl carnitine, tryptophan, linolenic acid, margaric acid, palmitate, linoleic acid, serine, arginine, docosatetraenoic acid, stearate, 3-methyl-2oxo-butyric acid, palmitoleic acid. If BMI is included the order from most important to least important is: linoleoyl-LPC, 3-hydroxy-butyrate, betaine, creatine, threonine, palmitoyl-LPC, 2-hydroxybutyrate, oleoyl-LPC, glycine, oleic acid, decanoyl carnitine, glutamic acid, octanoyl carnitine, tryptophan, margaric acid, linolenic acid, BMI, palmitate, linoleic acid, serine, stearate, docosatetraenoic acid, arginine, 3-methyl-2-oxo-butyric acid, palmitoleic acid.

TABLE 26
Confusion Matrix to Predict Progression to Dysglycemia without
(Top) or with (Bottom) BMI as a variable.

| | Progressors | Non-Progressors | Error |
|---|---|---|---|
| Progressors | 53 | 29 | 0.35 |
| Non-Progressors | 308 | 534 | 0.36 |

OOB Estimate of Error 36.47%

| | | | |
|---|---|---|---|
| Progressors | 53 | 29 | 0.35 |
| Non-Progressors | 311 | 531 | 0.37 |

OOB Estimate of Error 36.8%

The results were also analyzed using the t-test to determine the most significant biomarkers for predicting subjects that will progress to dysglycemia.

These results are presented in Table 27.

TABLE 27
T-test results of biomarkers for predicting progression to dysglycemia.

| | | | Non- | | |
|---|---|---|---|---|---|
| | | | Prog- | Prog- | |
| | | | ressors | ressors | |
| Biomarker | p-value | q-value | (Mean) | (Mean) | RATIO |
| linoleoyl-LPC | 1.38E−05 | 0.0002 | 16.33 | 13.55 | 0.83 |
| 2-hydroxybutyrate | 0.0018 | 0.0128 | 3.78 | 4.25 | 1.12 |
| oleoyl-LPC | 0.0034 | 0.0160 | 8.65 | 7.77 | 0.90 |
| serine | 0.0062 | 0.0218 | 10.45 | 9.80 | 0.94 |
| creatine | 0.0169 | 0.0344 | 3.89 | 4.56 | 1.17 |
| BMI | 0.0170 | 0.0344 | 25.15 | 26.26 | 1.04 |
| glutamic_acid | 0.0173 | 0.0344 | 14.15 | 16.22 | 1.15 |
| palmitate | 0.0218 | 0.0377 | 30.07 | 33.20 | 1.10 |
| glycine | 0.0244 | 0.0377 | 23.03 | 21.44 | 0.93 |
| oleate | 0.0382 | 0.0532 | 78.03 | 84.24 | 1.08 |
| linolenic_acid | 0.0530 | 0.0671 | 2.77 | 3.04 | 1.10 |
| arginine | 0.0679 | 0.0767 | 12.55 | 13.23 | 1.05 |
| palmitoyl-LPC | 0.0715 | 0.0767 | 32.87 | 30.93 | 0.94 |
| palmitoleic_acid | 0.2391 | 0.2380 | 3.72 | 4.17 | 1.12 |
| margaric_acid | 0.2780 | 0.2583 | 0.38 | 0.40 | 1.03 |
| betaine | 0.3009 | 0.2621 | 3.87 | 3.75 | 0.97 |
| docosatetraenoic_acid | 0.3231 | 0.2649 | 0.19 | 0.21 | 1.07 |
| stearate | 0.3916 | 0.2997 | 11.16 | 11.45 | 1.03 |
| 3-methyl-2-oxo- | 0.4086 | 0.2997 | 1.53 | 1.57 | 1.02 |
| butyric acid | | | | | |
| threonine | 0.4518 | 0.3122 | 14.72 | 14.87 | 1.01 |
| tryptophan | 0.4749 | 0.3122 | 11.18 | 11.27 | 1.01 |
| 3-hydroxy-butyrate | 0.4927 | 0.3122 | 6.91 | 5.82 | 0.84 |
| decanoyl_carnitine | 0.7983 | 0.4838 | 0.06 | 0.05 | 0.92 |
| octanoyl_carnitine | 0.9311 | 0.5407 | 0.03 | 0.03 | 0.93 |
| linolenic_acid | 0.9758 | 0.5440 | 15.78 | 15.62 | 0.99 |

The results of the Random Forest analysis show that measuring the biomarkers in baseline samples can predict the subjects that will progress to dyslipidemia at 3 years with >60% accuracy with or without including BMI in the analysis. The results are shown in the confusion matrix in Table 28. The RF analysis also orders the biomarkers from most important to least important to distinguish the subjects that will progress to dyslipidemia from those who will not progress to dyslipidemia. The order from most important to least important is: 3-hydroxy-butyrate, docosatetraenoic acid, linoleic acid, oleic acid, palmitoleic acid, octanoyl carnitine, palmitate, decanoyl carnitine, linolenic acid, stearate, tryptophan, glutamic acid, betaine, arginine, glycine, oleoyl-LPC, margaric acid, palmitoyl-LPC, threonine, serine, linoleoyl-LPC, 2-hydroxybutyrate, creatine, 3-methyl-2-oxo-butyric acid. If BMI is included the order from most important to least important is: docosatetraenoic acid, 3-hydroxybutyrate, oleic acid, linoleic acid, palmitoleic acid, octanoyl carnitine, decanoyl carnitine, linolenic acid, tryptophan, palmitate, stearate, arginine, glycine, palmitoyl-LPC, oleoyl-LPC, betaine, glutamic acid, margaric acid, threonine, serine, linoleoyl-LPC, BMI, 2-hydroybutyrate, creatine, 3-methyl-2-oxo-butyric acid.

TABLE 28
Confusion Matrix to Predict Progression to Dyslipidemia without
(Top) or with (Bottom) BMI as a variable.

| | Non-Progressors | Progressors | Error |
|---|---|---|---|
| Non-Progressors | 483 | 313 | 0.3932 |
| Progressors | 23 | 46 | 0.3333 |

OOB Estimate of Error 38.84%

| | | | |
|---|---|---|---|
| Non-Progressors | 483 | 313 | 0.3932 |
| Progressors | 23 | 46 | 0.3333 |

OOB Estimate of Error 38.84%

The results were also analyzed using the t-test to determine the most significant biomarkers for predicting subjects that will progress to dyslipidemia. These results are presented in Table 29.

TABLE 29
T-test results of biomarkers for predicting progression to dyslipidemia.

| | | | Non- | | |
|---|---|---|---|---|---|
| | | | Prog- | Prog- | |
| | | | ressor | ressor | |
| Biomarker | p-value | q-value | (Mean) | (Mean) | RATIO |
| palmitoleic_acid | 9.15E−05 | 0.0013 | 4.12 | 2.80 | 0.68 |
| betaine | 0.0064 | 0.0372 | 3.75 | 4.26 | 1.14 |
| linolenic_acid | 0.0079 | 0.0372 | 2.99 | 2.41 | 0.81 |
| BMI | 0.0384 | 0.1354 | 25.04 | 25.87 | 1.03 |
| oleic_acid | 0.0562 | 0.1534 | 82.81 | 72.76 | 0.88 |
| glycine | 0.0756 | 0.1534 | 23.07 | 21.61 | 0.94 |
| palmitate | 0.0815 | 0.1534 | 31.66 | 28.48 | 0.90 |
| 3-methyl-2-oxo- | 0.0869 | 0.1534 | 1.51 | 1.56 | 1.03 |
| butyric_acid | | | | | |
| 3-hydroxy-butyrate | 0.1384 | 0.1933 | 7.39 | 6.40 | 0.87 |
| creatine | 0.1499 | 0.1933 | 4.19 | 3.78 | 0.90 |
| glutamic_acid | 0.1506 | 0.1933 | 14.17 | 15.58 | 1.10 |
| octanoyl_carnitine | 0.2147 | 0.2424 | 0.03 | 0.03 | 1.06 |
| oleoyl-LPC | 0.2487 | 0.2424 | 8.57 | 8.11 | 0.95 |
| stearate | 0.2552 | 0.2424 | 11.59 | 10.90 | 0.94 |
| decanoyl_carnitine | 0.2576 | 0.2424 | 0.05 | 0.06 | 1.06 |
| serine | 0.2775 | 0.2448 | 10.38 | 10.07 | 0.97 |
| palmitoyl-LPC | 0.4232 | 0.3514 | 32.24 | 31.18 | 0.97 |
| tryptophan | 0.4767 | 0.3738 | 11.00 | 11.18 | 1.02 |
| margaric_acid | 0.5117 | 0.3792 | 0.40 | 0.38 | 0.96 |
| arginine | 0.5485 | 0.3792 | 12.75 | 12.94 | 1.01 |
| linoleoyl-LPC | 0.5642 | 0.3792 | 15.83 | 15.95 | 1.01 |
| 2-hydroxybutyrate | 0.7579 | 0.4863 | 3.88 | 3.91 | 1.01 |
| threonine | 0.8905 | 0.5095 | 14.76 | 14.78 | 1.00 |
| linoleic_acid | 0.8928 | 0.5095 | 16.18 | 15.93 | 0.98 |
| docosatetraenoic_acid | 0.9024 | 0.5095 | 0.20 | 0.20 | 0.99 |

While the invention has been described in detail and with reference to specific embodiments thereof, it will be apparent to one skilled in the art that various changes and modifications can be made without departing from the spirit and scope of the invention.

1-80. (canceled)

81. A method for diagnosing insulin resistance in a subject, the method comprising:
obtaining a biological sample from a subject;
analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, linoleoyl lysophosphatidylcholine, 1,5-anhydroglucitol, stearoyl-LPC, glutamyl valine, gamma-glutamyl-leucine, heptadecenoic acid, alpha-ketobutyrate, cysteine, urate, isovalerylcarnitine, myo-inositol, 1-palmitoyl-glycerophosphoethanolamine, catechol sulfate, and 3-phenylpropionate; and
comparing the level(s) of the one or more biomarkers in the sample to insulin resistance reference levels of the one or more biomarkers in order to diagnose whether the subject has insulin resistance.

82. The method of claim 81, wherein the method further comprises determining the subject's measurements of fasting plasma insulin, fasting plasma glucose, fasting plasma pro-insulin, fasting free fatty acids, HDL-cholesterol, LDL-cholesterol, C-peptide, adiponectin, peptide YY, hemoglobin A1C, waist circumference, body weight, or body mass index.

83. The method of claim 81, wherein the level(s) of the one or more biomarker(s) are analyzed using a method selected from the group consisting of mass-spectrometry (MS), tandem-mass-spectrometry (MS-MS), high performance liquid chromatography (HPLC), ELISA, nuclear magnetic resonance (NMR) spectroscopy, infrared (IR) spectroscopy, gas chromatography (GC), enzyme assay, and combinations thereof.

84. The method of claim 81, wherein reference levels are correlated to levels of glucose disposal as measured by hyperinsulemic euglycemic (HI) clamp.

85. The method of claim 81, wherein the biological sample is a urine sample, a blood sample, a plasma sample or a tissue sample.

86. A method of classifying a subject as having normal insulin sensitivity or being insulin resistant, the method comprising:
analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, linoleoyl lysophosphatidylcholine, 1,5-anhydroglucitol, stearoyl-LPC, glutamyl valine, gamma-glutamyl-leucine, heptadecenoic acid, alpha-ketobutyrate, cysteine, urate, isovalerylcarnitine, myo-inositol, 1-palmitoyl-glycerophosphoethanolamine, catechol sulfate, and 3-phenylpropionate; and
comparing the level(s) of the one or more biomarkers in the sample to glucose disposal rate reference levels of the one or more biomarkers in order to classify the subject as having normal insulin sensitivity or being insulin resistant.

87. The method of claim 86, wherein the comparing step comprises generating an insulin resistance score for the subject in order to classify the subject as having normal insulin sensitivity or being insulin resistant.

88. A method of determining the probability of a subject developing type-2 diabetes, the method comprising:
analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, linoleoyl lysophosphatidylcholine, 1,5-anhydroglucitol, stearoyl-LPC, glutamyl valine, gamma-glutamyl-leucine, heptadecenoic acid, alpha-ketobutyrate, cysteine, urate, isovalerylcarnitine, myo-inositol, 1-palmitoyl-glycerophosphoethanolamine, catechol sulfate, and 3-phenylpropionate; and
comparing the level(s) of the one or more biomarkers in the sample to diabetes-positive and/or -diabetes-negative reference levels of the one or more biomarkers in order to determine the probability of the subject developing type-2 diabetes.

89. The method of claim 88, wherein the comparing step comprises generating an insulin resistance score for the subject.

90. A method of monitoring the progression or regression of insulin resistance in a subject, the method comprising:
analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, linoleoyl lysophosphatidylcholine, 1,5-anhydroglucitol, stearoyl-LPC, glutamyl valine, gamma-glutamyl-leucine, heptadecenoic acid, alpha-ketobutyrate, cysteine, urate, isovalerylcarnitine, myo-inositol, 1-palmitoyl-glycerophosphoethanolamine, catechol sulfate, and 3-phenylpropionate; and
comparing the level(s) of the one or more biomarkers in the sample to insulin resistance progression and/or insulin resistance-regression reference levels of the one or more biomarkers in order to monitor the progression or regression of insulin resistance in the subject.

91. The method of claim 90, wherein the subject is selected from the group consisting of a subject being treated with a pharmaceutical composition, a subject having undergone bariatric surgery, a subject undergoing an exercise modification, and a subject using a dietary modification.

92. The method of claim 90, wherein the comparing step comprises generating an insulin resistance score for the subject in order to monitor the progression or regression of insulin resistance in the subject.

93. A method of monitoring the efficacy of insulin resistance treatment, the method comprising:
analyzing a first biological sample from a subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, linoleoyl lysophosphatidylcholine, 1,5-anhydroglucitol, stearoyl-LPC, glutamyl valine, gamma-glutamyl-leucine, heptadecenoic acid, alpha-ketobutyrate, cysteine, urate, isovalerylcarnitine, myo-inositol, 1-palmitoyl-glycerophosphoethanolamine, catechol sulfate, and 3-phenylpropionate;
treating the subject for insulin resistance;
analyzing a second biological sample from the subject to determine the level(s) of the one or more biomarkers, the second sample obtained from the subject at a time point after treatment; and
comparing the level(s) of one or more biomarkers in the first sample to the level(s) of the one or more biomarkers in the second sample to assess the efficacy of the treatment for treating insulin resistance.

94. The method of claim 93, wherein the subject is treated by a method selected from the group consisting of administration of a therapeutic agent, a dietary change, an exercise program change, a surgical procedure, and combinations thereof.

95. The method of claim 93, wherein the comparing step comprises generating an insulin resistance score for the subject in order to assess the efficacy of the treatment for insulin resistance.

96. A method for predicting a subject's response to a course of treatment for insulin resistance, the method comprising:
analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, linoleoyl lysophosphatidylcholine, 1,5-anhydroglucitol, stearoyl-LPC, glutamyl valine, gamma-glutamyl-leucine, heptadecenoic acid, alpha-ketobutyrate, cysteine, urate, isovalerylcarnitine, myo-inositol, 1-palmitoyl-glycerophosphoethanolamine, catechol sulfate, and 3-phenylpropionate; and
comparing the level(s) of one or more biomarkers in the sample to treatment-positive and/or treatment-negative reference levels of the one or more biomarkers to predict whether the subject is likely to respond to a course of treatment.

97. A method for monitoring a subject's response to a course of treatment for insulin resistance, the method comprising:
analyzing a first biological sample from a subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, linoleoyl lysophosphatidylcholine, 1,5-anhydroglucitol, stearoyl-LPC, glutamyl valine, gamma-glutamyl-leucine, heptadecenoic acid, alpha-ketobutyrate, cysteine, urate, isovalerylcarnitine, myo-inositol, 1-palmitoyl-glycerophosphoethanolamine, catechol sulfate, and 3-phenylpropionate;
treating the subject for insulin resistance;
analyzing a second biological sample from the subject to determine the level(s) of the one or more biomarkers, the second sample obtained from the subject at a time point after treatment;
comparing the level(s) of one or more biomarkers in the first sample to the level(s) of the one or more biomarkers in the second sample to assess the efficacy of the treatment for treating insulin resistance.

98. The method of claim 97, wherein the comparing step comprises generating an insulin resistance score for the subject in order to monitor a subject's response to a course of treatment for insulin resistance.

99. A method for determining a subject's probability of being insulin resistant, the method comprising:
obtaining a biological sample from a subject;
analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, linoleoyl lysophosphatidylcholine, 1,5-anhydroglucitol, stearoyl-LPC, glutamyl valine, gamma-glutamyl-leucine, heptadecenoic acid, alpha-ketobutyrate, cysteine, urate, isovalerylcarnitine, myo-inositol, 1-palmitoyl-glycerophosphoethanolamine, catechol sulfate, and 3-phenylpropionate,
predicting the glucose disposal rate in the subject by comparing the level(s) of the one or more biomarkers in the sample to glucose disposal rate reference levels of the one or more biomarkers;
comparing the predicted glucose disposal rate to an algorithm for insulin resistance based on the one or more markers; and
determining the probability that the subject is insulin resistant, thereby producing an insulin resistance score.

100. A method of identifying an agent capable of modulating the level of a biomarker of insulin resistance, the method comprising:
analyzing a cell line from a subject at a first time point to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, linoleoyl lysophosphatidylcholine, 1,5-anhydroglucitol, stearoyl-LPC, glutamyl valine, gamma-glutamyl-leucine, heptadecenoic acid, alpha-ketobutyrate, cysteine, urate, isovalerylcarnitine, myo-inositol, 1-palmitoyl-glycerophosphoethanolamine, catechol sulfate, and 3-phenylpropionate;
contacting the cell line with a test agent;
analyzing the cell line at a second time point to determine the level(s) of the one or more biomarkers, the second time point being a time after contacting with the test agent;
comparing the level(s) of one or more biomarkers in the cell line at the first time point to the level(s) of the one or more biomarkers in the cell line at the second time point to identify an agent capable of modulating the level of the one or more biomarkers.

101. An agent identified by the method of claim 100.

102. A method for measuring insulin resistance in a subject, the method comprising:
obtaining a biological sample from a subject;
analyzing the biological sample from the subject to determine the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, linoleoyl lysophosphatidylcholine, 1,5-anhydroglucitol, stearoyl-LPC, glutamyl valine, gamma-glutamyl-leucine, heptadecenoic acid, alpha-ketobutyrate, cysteine, urate, isovalerylcarnitine, myo-inositol, 1-palmitoyl-glycerophosphoethanolamine, catechol sulfate, and 3-phenylpropionate; and
using the determined levels of the level(s) of the one or more biomarkers and a reference model based on the one or more biomarkers to measure the insulin resistance in the subject.

103. The method of claim 102, wherein the comparing step comprises generating an insulin resistance score for the subject in order to classify the subject as having normal insulin sensitivity or being insulin resistant.

104. A method of treating an insulin resistant subject, the method comprising:
administering to the subject a therapeutic agent capable of modulating the level(s) of one or more biomarkers selected from the group consisting of 2-hydroxybutyrate, decanoyl carnitine, octanoyl carnitine, 3-hydroxy-butyrate, 3-methyl-2-oxo-butyric acid, arginine, betaine, creatine, docosatetraenoic acid, glutamic acid, glycine, linoleic acid, linolenic acid, margaric acid, oleic acid, oleoyl lysophosphatidylcholine, palmitate, palmitoleic acid, palmitoyl lysophosphatidylcholine, serine, stearate, threonine, tryptophan, linoleoyl lysophosphatidylcholine, 1,5-anhydroglucitol, stearoyl-LPC, glutamyl valine, gamma-glutamyl-leucine, heptadecenoic acid, alpha-ketobutyrate, cysteine, urate, isovalerylcarnitine, myo-inositol, 1-palmitoyl-glycerophosphoethanolamine, catechol sulfate, and 3-phenylpropionate, and one or more biochemicals and/or metabolites in a pathway related to the one or more biomarkers.
